SEQUENCE LISTING (5 sequences)

SEQ ID NO: 1 - protein, 391 aa, synthetic construct
MGGRDIVDGSEFSPSPVPGSQNVPAPAVKKISQYACQRRTTLNNYNQLFTDALDILAENDELRENEGSALAFMRASSVLKSLPFPITSMKDTEGIPSLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYADILESTFEKFKQPSRKVDALDHFQKCFLILKLDHGRVHSEKSGQQEGKGWKAIRVDLVMSPYDRRAFALLGWTGSRQFERDLRRYATHERKMMLDNHALYDRTKRVFLEAESEEEIFAHLGLDYIEPWERNA

SEQ ID NO: 2 - protein, 6 aa, synthetic construct
HHHHHH

SEQ ID NO: 3 - DNA, 10 bp, synthetic construct
  source 1..10: mol_type = other DNA
tttttttttt

SEQ ID NO: 4 - DNA, 100 bp, synthetic construct
  source 1..100: mol_type = other DNA
tttttttttttttttttttttttttttttttttttttttttttttttttttttttttttttttttttttttttttttttttttttttttttttttttttt

SEQ ID NO: 5 - DNA, 200 bp, synthetic construct
  source 1..200: mol_type = other DNA
tttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttt

(19) United States
(12) Patent Application Publication
(10) Pub. No.: US 2026/0132223 A1
(43) Pub. Date: May 14, 2026

(54) CLEAVABLE LINKERS FOR THE TETHERING OF POLYMERASES TO NUCLEOTIDES

(71) Applicant: Ansa Biotechnologies, Inc., Emeryville, CA (US)

(72) Inventors: Aaron Feldman, San Diego, CA (US); Sebastian Palluk, Oakland, CA (US); Daniel Arlow, San Francisco, CA (US); Jeffrey George Bertram, Montebello, CA (US); Ben Raliski, Emeryville, CA (US); Ronald T. Raines, Cambridge, MA (US); Uwe Theo Bornscheuer, Greifswald (DE); Nico Dennis Fessner, Greifswald (DE); Christoffel Petrus Stephanus Badenhorst, Greifswald (DE)

(21) Appl. No.: 19/091,129

(22) Filed: Mar. 26, 2025

Related U.S. Application Data
(63) Continuation of application No. PCT/US2023/075020, filed on Sep. 25, 2023.
(60) Provisional application No. 63/377,121, filed on Sep. 26, 2022.

(51) Int. Cl. C07K 19/00; C07H 19/10; C07H 19/20; C12N 9/12; C12Q 1/6806

(52) U.S. Cl. CPC C07K 19/00; C07H 19/10; C07H 19/20; C12N 9/1264; C12Q 1/6806

(57) ABSTRACT

The present disclosure provides methods of polynucleotide synthesis and compounds, compositions useful for synthesis of polynucleotides. The chemical compounds include nucleotides and their analogs that are attached to polymerase though a cleavable linker comprising an enzymatically cleavable amino acid ester.

### CROSS REFERENCE TO RELATED APPLICATIONS

This application is a bypass continuation of PCT/US2023/075020, filed Sep. 25, 2023, which claims priority to U.S. Provisional Patent Application No. 63/377,121, filed Sep. 26, 2022; the contents of which are hereby incorporated by reference herein in its entirety.

### GOVERNMENT SUPPORT STATEMENT

This invention was made with government support under SBIR Award Numbers 1914354 and 2036532 awarded by the National Science Foundation (NSF). The government has certain rights in the invention.

### REFERENCE TO A SEQUENCE LISTING

The instant application contains a Sequence Listing (XML file named ABB-009WOC1 Sequence Listing.xml, generated on Nov. 15, 2023 and 6,091 bytes in size), which has been submitted electronically and is incorporated by reference herein.

### BACKGROUND

The majority of biological research and bioengineering involves synthetic DNA, which can include oligonucleotides, synthetic genes, or even chromosomes. Synthetic DNA is custom-built using the phosphoramidite method. Unfortunately, phosphoramidite synthesis cannot produce high-quality long oligos. Each cycle of synthesis induces a low but detectable level of depurination, branching, and other types of lesions in the nascent oligo, which cause errors in the final sequence. This error rate is negligible for short oligos used in low-throughput applications (e.g., PCR primers or Sanger sequencing), but it becomes significant with increasing length and throughput. Thus, the maximum oligo length achievable through phosphoramidite synthesis is limited to ˜200 bp.

An enzymatic method of single-stranded DNA (ssDNA) synthesis has long been sought after as an alternative to phosphoramidite chemistry. TdT is capable of polymerizing thousands of non-templated nucleotides into a strand of DNA, but synthesis of a defined sequence demands that incorporation be strictly limited to one base at a time. In next generation sequencing (NGS), this function is provided by nucleotides with a removable 3′ blocking group (“reversible terminator”), but TdT does not readily incorporate NTPs containing functional groups that block elongation.

To achieve stepwise, controlled enzymatic DNA synthesis, a conjugate of TdT bound to a nucleotide via a cleavable linker can be used. When exposed to the free 3′ end of an oligonucleotide, the conjugate adds its tethered nucleotide and remains attached to the extended primer, blocking further elongation by other conjugates. The linker is then cleaved to release the TdT and expose the end of the oligo for the next extension. These two steps of “extension” and “deprotection” are iterated to synthesize a defined sequence.

However, if a cleavable linker spontaneously cleaves before the controlled cleavage step, it can lead to undesired insertions during oligo synthesis, due to the presence of free nucleotide and polymerase in the added conjugate solution. Furthermore, a linker that does not cleave during the controlled cleavage step can result in one or more unwanted deletions during synthesis, as no new nucleotide can be added while the polymerase remains bound.

What is needed, therefore, are improved linkers that are highly stable under storage and oligo synthesis reaction conditions, but are quantitatively cleaved in a short timeframe, while leaving only a benign residue, or “scar”, on the base.

### SUMMARY

In some embodiments, provided herein is a conjugate comprising a polymerase, a nucleotide and a cleavable linker attached to the polymerase and the nucleotide, wherein the cleavable linker comprises an amino acid ester. In some embodiments, the amino acid ester is attached to an amino acid. In some embodiments, the amine group of the amino acid ester is bound to the amino acid. In some embodiments, the conjugate comprises a peptide of at least 2, at least 3, at least 4, or at least 5 amino acids bound to the amine group of the amino acid ester.

In some embodiments, the amino acid or amino acids is selected from the group consisting of: alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In some embodiments, the amino acid is glycine or the amino acids comprise glycine. In some embodiments, the amino acid is a non-naturally occurring amino acid or the amino acids comprise a non-naturally occurring amino acid.

In some embodiments, the cleavable linker is bound to the alpha-phosphate, sugar, or nucleobase of the nucleotide.

In some embodiments, the amino acid ester is represented by:

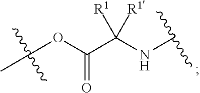

wherein R¹ and R^{1′} are each independently selected from hydrogen and an optionally substituted C₁₋₆ alkyl, or are optionally taken together with the atom on which they are attached to form an optionally substituted C₃-C₇ carbocyclic ring.

In some embodiments, the amino acid ester is represented by a compound selected from the group consisting of:

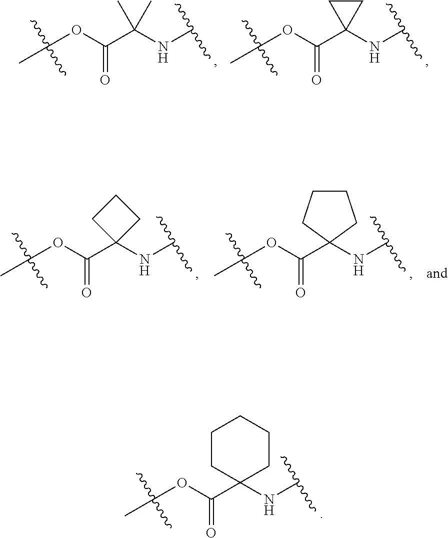

In some embodiments, the linker comprises the structure:

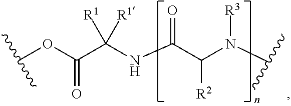

wherein R¹ and R^{1′} are each independently selected from hydrogen and an optionally substituted C₁₋₆ alkyl or are optionally taken together with the atom on which they are attached to form an optionally substituted C₃-C₇ carbocyclic ring; each R² is an optionally substituted group independently selected from the group consisting of hydrogen, C₁₋₆ alkyl, phenyl, C₁-C₆ carbocyclic ring and 3-7 heterocyclic ring; each R³ is hydrogen or optionally substituted C₁₋₆ alkyl; and n is 1, 2, 3, 4 or 5. In some embodiments, R³ is hydrogen. In some embodiments, R² is hydrogen. In some embodiments, R² is selected from the group consisting of hydrogen, -Me, -iso-Pr, -sec-butyl, iso-butyl, —CH₂Ph, —CH₂OH, —CH₂SH, —CH₂CH₂SCH₃, —CH₂COOH, —CH₂CH₂COOH, —CH₂CONH₂, —CH₂CH₂CONH₂, —CH₂CH₂, CH₂CH₂NH₂,

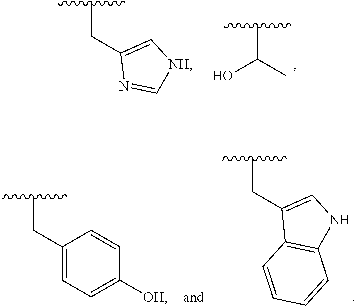

In some embodiments, n is 1. In some embodiments, R¹ and R^{1′} are taken together to form an optionally substituted C₃-C₇ carbocyclic ring. In some embodiments, R¹ and R^{1′} are taken together to form an optionally substituted C₃ carbocyclic ring.

In some embodiments, the linker comprises the structure:

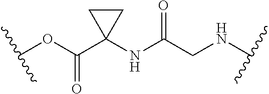 [SMILES: *NCC(=O)NC1(C(=O)O*)CC1]

In some embodiments, the conjugate comprises the structure:

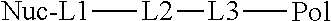

wherein Nuc is the nucleotide; Pol is the polymerase; L1 is a first portion of the linker connecting the nucleotide to L2; L2 is a second portion of the linker represented by:

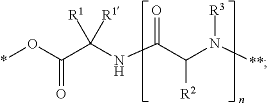

wherein R¹ and R^{1′} are each independently selected from an optionally substituted C₁₋₆ alkyl, a halogen, or are optionally taken together with the atom on which they are attached to form an optionally substituted C₃-C₇ carbocyclic ring; each R² is an optionally substituted group independently selected from the group consisting of hydrogen, C₁₋₆ alkyl, phenyl, C₁-C₆ carbocyclic ring and 3-7 heterocyclic ring; each R³ is hydrogen or optionally substituted C₁₋₆ alkyl; n is 0, 1, 2, 3, 4 or 5; wherein * indicates the attachment point of L2 to L1; and ** indicates the attachment point of L2 to L3; wherein L² is cleavable; and L³ is a linker connecting pol to L².

In some embodiments, L¹ is selected from the group consisting of a bond, an optionally substituted C₁₋₁₂ alkylene chain, C₄-C₂₀ polyethylene glycol, an optionally substituted C₂₋₁₂ alkenylene chain, and a C₂₋₁₂ alkynylene chain, wherein 1-6 methylene units of L¹ are optionally and independently replaced with —O—, —N(R^{b})—, —N═C(H)—, —C(O)—, —S—, —S(O)—, —S(O)₂—, optionally substituted phenylene, or optionally substituted cyclopropylene.

In some embodiments, L¹ comprises:

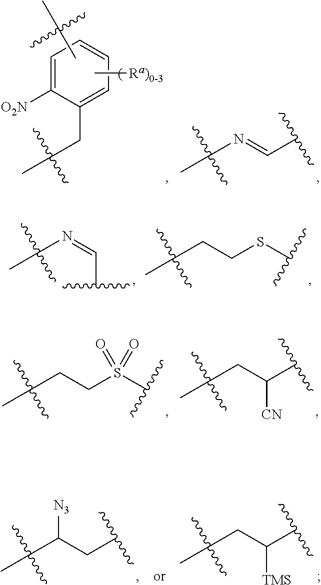

wherein each Ra is independently selected from the group consisting of halogen, hydroxyl, cyano, optionally substituted C₁₋₆ alkyl, and optionally substituted C₁₋₆ alkoxy.

In some embodiments, L² comprises an amino acid ester selected from the group consisting of:

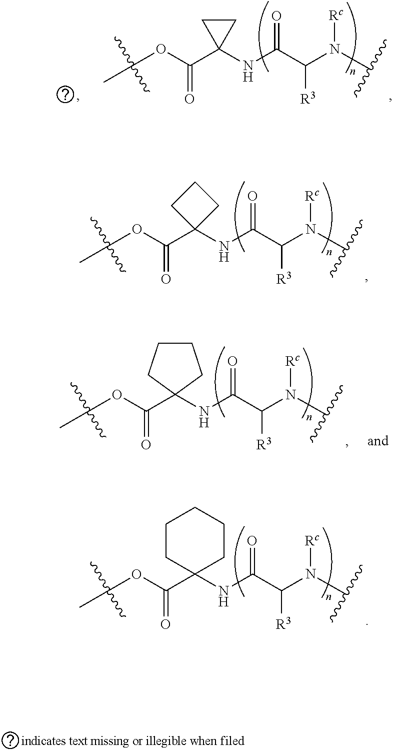

In some embodiments, L² is represented by:

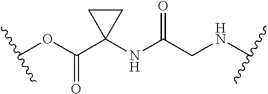 [SMILES: *NCC(=O)NC1(C(=O)O*)CC1]

In some embodiments, L¹ is bound to the nucleobase of the nucleotide. In some embodiments, L¹ is bound to the nucleobase at an oxygen or nitrogen involved in base pairing. In some embodiments, the nucleobase is selected from the group consisting of:

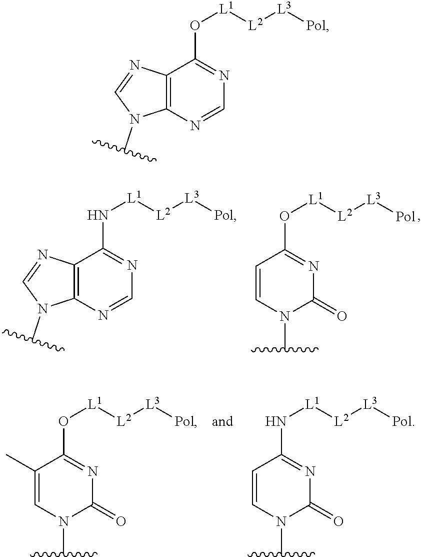

In some embodiments, L¹ is bound to the sugar of the nucleotide.

In some embodiments, L¹ is bound to a phosphate of the nucleotide. In some embodiments, the phosphate is the alpha phosphate. In some embodiments, the nucleotide is a ribonucleotide polyphosphate or a deoxyribonucleotide polyphosphate. In some embodiments, the nucleotide is selected from the group consisting of: adenine, guanine, cytosine, uracil, and thymine.

In some embodiments, the polymerase is a template-independent polymerase. In some embodiments, the polymerase is TdT.

In some embodiments, the linker is capable of being cleaved by a protease comprising esterase activity. In some embodiments, the linker is capable of being cleaved by Proteinase K. In some embodiments, linker is capable of being cleaved at the ester group on L2, leaving a compound represented by Nuc-L1-OH after said cleavage.

Also provided herein, in some embodiments, is a method of synthesizing a polynucleotide, comprising: incubating a polynucleotide with the conjugate described herein. In some embodiments, the method further comprises extending the polynucleotide by adding the nucleotide bound to said conjugate to 3′ OH of said polynucleotide.

In some embodiments, the method further comprises cleaving said cleavable linker after addition of said nucleotide to said precursor polynucleotide. In some embodiments, the method further comprises repeating said incubating, extending, and cleaving steps one or more times. In some embodiments, said cleaving comprises contacting said extended polynucleotide with an enzyme comprising esterase activity under conditions sufficient to cleave the linker, thereby releasing the polymerase from the extension product. In some embodiments, said enzyme is a protease comprising esterase activity.

In some embodiments, the method further comprises removing a scar attached to the nucleotide remaining after said cleavage of the linker.

In some embodiments, the scar removal is performed after completion of polynucleotide synthesis. In some embodiments, the scar removal is performed after synthesis of a portion of the polynucleotide. In some embodiments, the scar removal is performed after cleavage of the linker and before addition of the next nucleotide during polynucleotide synthesis.

Also provided herein, according to some embodiments, is a method of synthesizing a polynucleotide, comprising: (a) incubating a nucleic acid with a first conjugate as described herein under conditions in which the polymerase catalyzes the covalent addition of the nucleotide of the first conjugate onto the 3′ hydroxyl of the nucleic acid to make a first extension product; (b) cleaving the cleavable linkage of the linker, thereby releasing the polymerase from the extension product to de-shield the 3′ hydroxyl end of the first extension product; (c) incubating the extension product with a second conjugate as described herein under conditions in which the polymerase catalyzes the covalent addition of the nucleotide of the second conjugate onto the 3′ end of the first extension product, to make a second extension product; (d) repeating steps (b)-(c) on the second extension product multiple times to produce an extended nucleic acid of a defined sequence.

Also provided herein, according to some embodiments, is a method of sequencing, comprising: incubating a duplex comprising a primer and a template with a composition comprising a set of conjugates of any one of claims 1-36, wherein the conjugates correspond to G, A, T (or U) and C and are distinguishably labeled; detecting which nucleotide has been added to the primer by detecting a signal from said distinguishable label; cleaving the cleavable linkage of the linker, thereby releasing the polymerase from the extension product to de-shield the 3′ hydroxyl end of the first extension product; and repeating the incubation, detection and cleaving steps to determine a sequence of the template.

Also provided herein, in some embodiments, is a modified nucleotide comprising a cleavable linker, wherein the cleavable linker comprises an amino acid ester. In some embodiments, the amino acid ester is attached to an amino acid. In some embodiments, the amine group of the amino acid ester is bound to the amino acid. In some embodiments, the modified nucleotide comprises a peptide of at least 2, at least 3, at least 4, or at least 5 amino acids bound to the amine group of the amino acid ester. In some embodiments, the amino acid or amino acids is selected from the group consisting of: alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In some embodiments, the amino acid is glycine or the amino acids comprise glycine. In some embodiments, the amino acid is a non-naturally occurring amino acid or the amino acids comprise a non-naturally occurring amino acid. In some embodiments, the cleavable linker is bound to the alpha-phosphate, sugar, or nucleobase of the nucleotide.

In some embodiments, the amino acid ester is represented by:

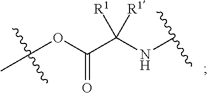

wherein R¹ and R^{1′} are each independently selected from hydrogen and an optionally substituted C₁₋₆ alkyl, or are optionally taken together with the atom on which they are attached to form an optionally substituted C₃-C₇ carbocyclic ring.

In some embodiments, the amino acid ester is represented by a compound selected from the group consisting of:

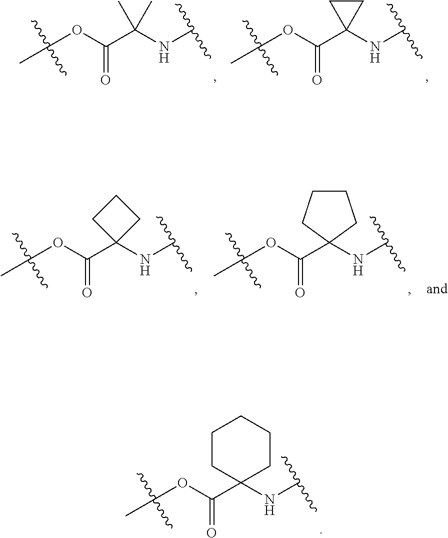

In some embodiments, the linker comprises the structure:

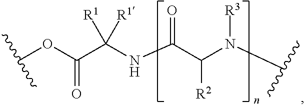

wherein R¹ and R^{1′} are each independently selected from hydrogen and an optionally substituted C₁₋₆ alkyl or are optionally taken together with the atom on which they are attached to form an optionally substituted C₃-C₇ carbocyclic ring; each R² is an optionally substituted group independently selected from the group consisting of hydrogen, C₁₋₆ alkyl, phenyl, C₁-C₆ carbocyclic ring and 3-7 heterocyclic ring; each R³ is hydrogen or optionally substituted C₁₋₆ alkyl; and n is 1, 2, 3, 4 or 5. In some embodiments, R³ is hydrogen. In some embodiments, R² is hydrogen. In some embodiments, R² is selected from the group consisting of hydrogen, -Me, -iso-Pr, -sec-butyl, iso-butyl, —CH₂Ph, —CH₂OH, —CH₂SH, —CH₂CH₂SCH₃, —CH₂COOH, —CH₂CH₂COOH, —CH₂CONH₂, —CH₂CH₂CONH₂, —CH₂CH₂, CH₂CH₂NH₂,

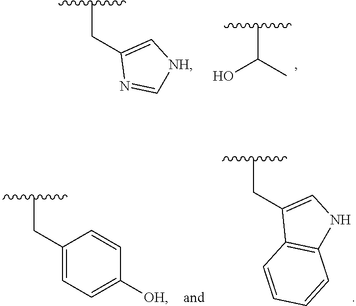

In some embodiments, n is 1. In some embodiments, R¹ and R^{1′} are taken together to form an optionally substituted C₃-C₇ carbocyclic ring. In some embodiments, R¹ and R^{1′} are taken together to form an optionally substituted C₃ carbocyclic ring.

In some embodiments, the linker comprises the structure:

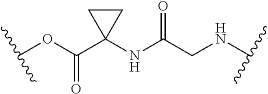 [SMILES: *NCC(=O)NC1(C(=O)O*)CC1]

In some embodiments, the conjugate comprises the structure:

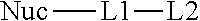

wherein Nuc is the nucleotide; L1 is a first portion of the linker connecting the nucleotide to L2; L2 is a second portion of the linker represented by:

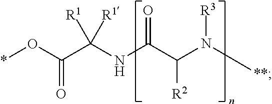

wherein R¹ and R^{1′} are each independently selected from an optionally substituted C₁₋₆ alkyl, a halogen, or are optionally taken together with the atom on which they are attached to form an optionally substituted C₃-C₇ carbocyclic ring; each R² is an optionally substituted group independently selected from the group consisting of hydrogen, C₁₋₆ alkyl, phenyl, C₁-C₆ carbocyclic ring and 3-7 heterocyclic ring; each R³ is hydrogen or optionally substituted C₁₋₆ alkyl; n is 0, 1, 2, 3, 4 or 5; wherein * indicates the attachment point of L2 to L1; and wherein L² is cleavable.

In some embodiments, L¹ is selected from the group consisting of a bond, an optionally substituted C₁₋₁₂ alkylene chain, C₄-C₂₀ polyethylene glycol, an optionally substituted C₂₋₁₂ alkenylene chain, and a C₂₋₁₂ alkynylene chain, wherein 1-6 methylene units of L¹ are optionally and independently replaced with —O—, —N(R^{b})—, —N═C(H)—, —C(O)—, —S—, —S(O)—, —S(O)₂—, optionally substituted phenylene, or optionally substituted cyclopropylene.

In some embodiments, L¹ comprises:

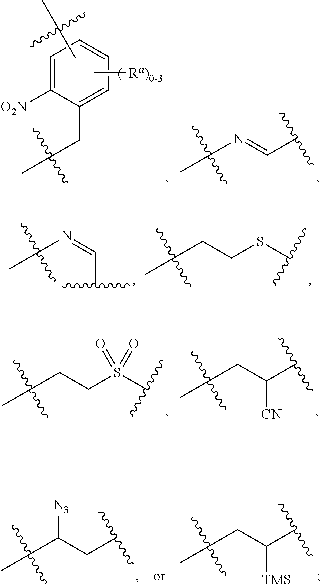

wherein each R^{a} is independently selected from the group consisting of halogen, hydroxyl, cyano, optionally substituted C₁₋₆ alkyl, and optionally substituted C₁₋₆ alkoxy.

In some embodiments, L² comprises an amino acid ester selected from the group consisting of:

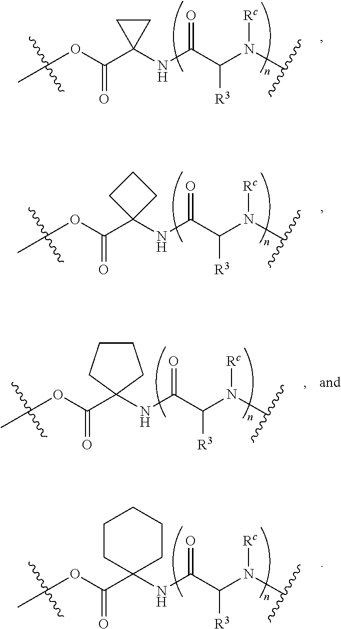

In some embodiments, L² is represented by:

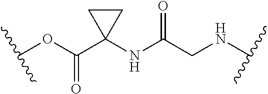 [SMILES: *NCC(=O)NC1(C(=O)O*)CC1]

In some embodiments, L¹ is bound to the nucleobase of the nucleotide. In some embodiments, L¹ is bound to the nucleobase at an oxygen or nitrogen involved in base pairing.

In some embodiments, L¹ is bound to the sugar of the nucleotide.

In some embodiments, L¹ is bound to a phosphate of the nucleotide. In some embodiments, the phosphate is the alpha phosphate. In some embodiments, the nucleotide is a ribonucleotide polyphosphate or a deoxyribonucleotide polyphosphate. In some embodiments, the nucleotide is selected from the group consisting of: adenine, guanine, cytosine, uracil, and thymine.

In some embodiments, the linker is capable of being cleaved by a protease comprising esterase activity. In some embodiments, the linker is capable of being cleaved by Proteinase K. In some embodiments, linker is capable of being cleaved at the ester group on L2, leaving a compound represented by Nuc-L1-OH after said cleavage.

In some embodiments, provided herein is a conjugate comprising a polymerase, a nucleotide and a cleavable linker attached to the polymerase and the nucleotide, wherein the cleavable linker is enzymatically cleavable. In some embodiments, the cleavable linker is capable of being cleaved by a protease. In some embodiments, provided herein is a modified nucleotide comprising a cleavable linker, wherein the cleavable linker is enzymatically cleavable. In some embodiments, the cleavable linker is capable of being cleaved by a protease.

### BRIEF DESCRIPTION OF THE DRAWINGS

The foregoing and other objects, features and advantages will be apparent from the following description of particular embodiments, as illustrated in the accompanying drawings in which like reference characters refer to the same parts throughout the different views. The drawings are not necessarily to scale, emphasis instead placed upon illustrating the principles of various embodiments.

FIG. 1 shows two amino acid ester dTTP analogs used for oligo synthesis and linker cleavage. One is based on a hydroxypropargyl scar (Linker 1) and the other on a smaller hydroxymethyl scar (Linker 2). The two amino acid ester dTTP analogs (Linkers 1 and 2, FIG. 1; synthesized by Jena Bioscience) were attached to cysteine-reactive crosslinkers and conjugated to TdT with the final structure shown in FIG. 1. Also shown is the alcohol-scarred cleavage products after ester cleavage of the linker.

FIGS. 2A, 2B, and 2C show a plot of the kinetics of conjugate addition to an unscarred oligo (FIGS. 2A and 2B)) and to a hydroxymethyl scarred oligo (FIG. 2C) FIG. 2A: Natural DNA primer exposed to a dTTP conjugate comprising an ester linkage for 1 second results in ˜35% extension yield. FIG. 2B: The oligo synthesis reaction proceeds to completion, with linker cleavage yielding a primer with a hydroxymethyl scar on the last base. FIG. 2C. Exposure of the scarred primer to the dTTP conjugate comprising an ester linkage for 1 second again results in ˜35% extension yield.

FIG. 3 shows the results of a primer extension by TdT-dTTP conjugates based on Linker 1 or Linker 2 as measured by a gel shift assay on SDS-PAGE. An ssDNA primer was extended for 60 s with 1) a Linker 1 conjugate, 2) a Linker 2 conjugate 3) a Linker 2 conjugate (replicates), 4) no conjugate. T/P: TdT/DNA primer complex. P: ssDNA primer.

FIG. 4 shows primer extension products as measured by capillary electrophoresis. Extension was performed by linker 2 conjugates stored overnight at the indicated pH, or in buffer only (negative control). Extension without insertion shows a peak at −58 nt. A peak indicating unwanted insertion (elongation products) is in some samples at −59 nt and indicates the presence of free dNTPs in the incubated conjugate.

FIG. 5 shows the results of an Enzymatic synthesis of 100mer and 200mer dT oligos using the linker 2 dNTP conjugate as measured using capillary electrophoresis (part A). An enlarged view of the product distributions of the 100 mers from enzymatic synthesis (top) and chemical synthesis (bottom) synthesis as observed via capillary electrophoresis is shown in part B.

FIG. 6 shows the results of an extension of an oligonucleotide using TdT-dATP, -dCTP, -dGTP, and -dTTP conjugates comprising linker 6 as measured by capillary electrophoresis (Panel A), and a cleavage time course of a TdT-dTTP conjugate comprising linker 6 incorporated into an oligonucleotide and cleaved via proteinase K for 30-240 seconds, as measured by capillary electrophoresis (Panel B).

FIG. 7 shows structures for a linker nucleotide comprising a glycine amino acid ester (Gly-OMe-U) and an ACC amino acid ester (ACC-OMe-U) and the product of ester instability of both linkers (HOMe-U) (top), and a comparison of the intact (Gly-OMe-U or ACC-OMe-U) and hydrolized (HOMe-U) product after 60 minutes of exposure to a temperature of 45° C.

FIGS. 8A, 8B and 8C show a comparison of the linker cleavage efficiency of various TdT-nucleotide conjugates. Data shown is at the time point for 60 seconds of ProK treatment.

FIG. 9 shows a series of electropherographs characterizing the cleavage rate by Proteinase K (ProK) for illustrative linkers having an aminocyclopropyl carboxy ethyl group and either one (1XG) or two (2XG) glycines. The cleavage reactions were quenched after 15 seconds (s), 30 s, 60 s, 4 minutes (m), 8 m, or 16 m.

FIG. 10 shows the results of conjugate addition to the primer 3.8 seconds after addition of the conjugate for each of the TdT-nucleotide conjugates (L2=ACC, Gly-ACC, or 2XGly-ACC).

FIG. 11 shows a plot of % ester hydrolysis for compounds 14-18 (ring expansion series linker nucleotides) after exposure to 50° C. from 1 minute to 20 hours.

FIG. 12 shows the results of exposure to a temperature of 50° C. for 1 hour, 4 hours, or overnight of an oligonucleotide extended with an Allyl G, ACC, AiB, AC4C, AC5C, or AC6C conjugate as measured by capillary electrophoresis to show proportion of intact and hydrolyzed products.

### DETAILED DESCRIPTION

The details of various embodiments of the present disclosure are set forth in the description below. Other features, objects, and advantages of the present disclosure will be apparent from the description and the drawings, and from the claims.

### Definitions

The term “alkyl” refers to a straight or branched full saturated hydrocarbon chain. Exemplary alkyl groups are methyl, ethyl, propyl, isopropyl, butyl, isobutyl, and tert-butyl.

The term “haloalkyl” refers to a straight or branched alkyl group that is substituted with one or more halogen atoms.

As described herein, compounds of the present disclosure may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)_{0- 4}N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{†}, -(haloR^{†}), —OH, —OR^{†}, —O(haloR^{†}), —CN, —C(O)OH, —C(O)OR^{†}, —NH₂, —NHR^{†}, —NR^{†}₂, or NO₂, wherein each R^{†} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

As used herein, the terms “about” and “approximately” refer to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition. Also, where ranges and/or subranges of values are provided, the ranges and/or subranges can include the endpoints of the ranges and/or subranges.

The terms “nucleic acid”, “polynucleotide” and “oligonucleotide” and other related terms used herein are used interchangeably and refer to polymers of nucleotides and are not limited to any particular length. Nucleic acids include recombinant and chemically-synthesized forms. Nucleic acids can be isolated. Nucleic acids include DNA molecules (e.g., cDNA or genomic DNA), RNA molecules (e.g., mRNA), analogs of the DNA or RNA generated using nucleotide analogs (e.g., peptide nucleic acids (PNA) and non-naturally occurring nucleotide analogs), and chimeric forms containing DNA and RNA. Nucleic acids can be single-stranded or double-stranded. Nucleic acids comprise polymers of nucleotides, where the nucleotides include natural or non-natural bases and/or sugars. Nucleic acids comprise naturally-occurring internucleosidic linkages, for example phosphdiester linkages. Nucleic acids can lack a phosphate group. Nucleic acids comprise non-natural internucleoside linkages, including phosphorothioate, phosphorothiolate, or peptide nucleic acid (PNA) linkages. In some embodiments, nucleic acids comprise a one type of polynucleotides or a mixture of two or more different types of polynucleotides.

The term “operably linked” and “operably joined” or related terms as used herein refers to juxtaposition of components. The juxtapositioned components can be linked together covalently. For example, two nucleic acid components can be enzymatically ligated together where the linkage that joins together the two components comprises phosphodiester linkage. A first and second nucleic acid component can be linked together, where the first nucleic acid component can confer a function on a second nucleic acid component. For example, linkage between a primer binding sequence and a sequence of interest forms a nucleic acid library molecule having a portion that can bind to a primer. In another example, a transgene (e.g., a nucleic acid encoding a polypeptide or a nucleic acid sequence of interest) can be ligated to a vector where the linkage permits expression or functioning of the transgene sequence contained in the vector. In some embodiments, a transgene is operably linked to a host cell regulatory sequence (e.g., a promoter sequence) that affects expression of the transgene. In some embodiments, the vector comprises at least one host cell regulatory sequence, including a promoter sequence, enhancer, transcription and/or translation initiation sequence, transcription and/or translation termination sequence, polypeptide secretion signal sequences, and the like. In some embodiments, the host cell regulatory sequence controls expression of the level, timing and/or location of the transgene.

The terms “linked”, “joined”, “attached”, “appended” and variants thereof comprise any type of fusion, bond, adherence or association between any combination of compounds or molecules that is of sufficient stability to withstand use in the particular procedure. The procedure can include but are not limited to: nucleotide binding; nucleotide incorporation; de-blocking (e.g., removal of chain-terminating moiety); washing; removing; flowing; detecting; imaging and/or identifying. Such linkage can comprise, for example, covalent, ionic, hydrogen, dipole-dipole, hydrophilic, hydrophobic, or affinity bonding, bonds or associations involving van der Waals forces, mechanical bonding, and the like. In some embodiments, such linkage occurs intramolecularly, for example linking together the ends of a single-stranded or double-stranded linear nucleic acid molecule to form a circular molecule. In some embodiments, such linkage can occur between a combination of different molecules, or between a molecule and a non-molecule, including but not limited to: linkage between a nucleic acid molecule and a solid surface; linkage between a protein and a detectable reporter moiety; linkage between a nucleotide and detectable reporter moiety; and the like. Some examples of linkages can be found, for example, in Hermanson, G., “Bioconjugate Techniques”, Second Edition (2008); Aslam, M., Dent, A., “Bioconjugation: Protein Coupling Techniques for the Biomedical Sciences”, London: Macmillan (1998); Aslam, M., Dent, A., “Bioconjugation: Protein Coupling Techniques for the Biomedical Sciences”, London: Macmillan (1998).

When used in reference to nucleic acids, the terms “extend”, “extending”, “extension” and other variants, refers to incorporation of one or more nucleotides into a nucleic acid molecule. Nucleotide incorporation comprises polymerization of one or more nucleotides into the terminal 3′ OH end of a nucleic acid strand (e.g., a nucleic acid primer), resulting in extension of the nucleic acid strand (e.g., extended primer). Nucleotide incorporation can be conducted with natural nucleotides and/or nucleotide analogs.

The terms “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. In embodiments, a cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents).

Use of the term “cleavable linker” is not meant to imply that the whole linker is required to be removed. The cleavage site can be located at a position on the linker that ensures that part of the linker remains attached to the dye and/or substrate moiety after cleavage. Cleavable linkers may be, by way of non-limiting example, electrophilically cleavable linkers, nucleophilically cleavable linkers, photocleavable linkers, cleavable under reductive conditions (for example disulfide or azide containing linkers), oxidative conditions, cleavable via use of safety-catch linkers and cleavable by elimination mechanisms. The use of a cleavable linker to attach the dye compound to a substrate moiety ensures that the label can, if required, be removed after detection, avoiding any interfering signal in downstream steps.

In embodiments, the cleavable linker is cleaved by contacting the cleavable linker with a cleaving agent (e.g., a reducing agent). In embodiments, the cleaving agent is . . . .

The term “polymerase-compatible cleavable moiety” and “polymerase-compatible cleavable linker” as used herein refers to a cleavable moiety or cleavable linker which does not interfere with the function of a polymerase (e.g., a DNA polymerase or modified DNA polymerase, in incorporating the nucleotide, to which the polymerase-compatible cleavable moiety is attached, to the 3′ end of the newly formed nucleotide strand). Methods for determining the function of a polymerase contemplated herein are described in B. Rosenblum et al. (Nucleic Acids Res. 1997 Nov. 15; 25(22): 4500-4504); and Z. Zhu et al. (Nucleic Acids Res. 1994 Aug. 25; 22(16): 3418-3422), which are incorporated by reference herein in their entirety for all purposes. In embodiments the polymerase-compatible cleavable moiety does not decrease the function of a polymerase relative to the absence of the polymerase-compatible cleavable moiety. In embodiments, the polymerase-compatible cleavable moiety does not negatively affect DNA polymerase recognition. In embodiments, the polymerase-compatible cleavable moiety does not negatively affect (e.g., limit) the read length of the DNA polymerase.

### Enzymatic Polynucleotide Synthesis

The present disclosure describes a method of enzymatic polynucleotide synthesis using polymerase-nucleotide conjugates to control the iterative addition of a single nucleotide per cycle onto the 3′ hydroxyl terminus of a growing polynucleotide strand via the nucleotide-bound polymerase to perform polynucleotide synthesis. Such control is achieved through a so-called “shielding effect”. Shielding describes the steric hinderance that prevents the 3′ hydroxyl terminus that has been elongated by a conjugate from being accessed by another conjugate while the polymerase remains attached to the added nucleotide, as well as the preventing the polymerase tethered to the nucleotide at the 3′ terminus from accessing the nucleotides of other conjugates.

Described in PCT Publication No. WO2017/223517, incorporated by reference in its entirety herein, is a typical process for the stepwise synthesis of a defined sequence using a template-independent polymerase. A nucleic acid that serves as an initial substrate for elongation (i.e. “starter molecule”) is incubated with a first polymerase-nucleotide conjugate. Once the nucleic acid has been elongated by the tethered nucleotide of a conjugate, no further elongations occur because the conjugates implement a termination mechanism. In the second step of the process, the linker is cleaved to release the polymerase and reverse the termination mechanism, thus enabling subsequent elongations. The elongation products are then exposed to the second conjugate, and these two steps are iterated to elongate the nucleic acid by a defined sequence. Also described in WO2017/223517 is a synthesis procedure using a conjugate comprising TdT and a photocleavable linker. As described above, other strategies are available for the attachment and cleavage of the linker.

An important step in this approach to polynucleotide synthesis is deprotection, or the removal of the tethered polymerase from the extended polynucleotide, making the 3′ terminus available for continued extension in the next cycle of synthesis. To be useful for polynucleotide synthesis, the removal of the tethered polymerase preferably occurs with rapid kinetics to reduce synthesis cycle time, while also being performed under benign conditions to prevent damage to the polynucleotide being synthesized. The removal of the tethered polymerase is also preferred to proceed to full completion, and to produce a cleavage product which does not impede continued extension or downstream applications of the complete DNA synthesis product. In some embodiments, the tether also allows for efficient conjugation of the nucleotide to the polymerase, and subsequently positions the nucleotide effectively within the active site to promote rapid incorporation to a free primer 3′ terminus.

Herein, we describe optimized cleavable linker designs used for the tethering of polymerases to nucleotides that are highly stable during storage and under oligo synthesis reaction conditions (before controlled linker cleavage), and enzymatically cleavable to completion in a short timeframe suitable for oligo synthesis.

### Cleavable Linker

Provided herein is a conjugate comprising a polymerase and a nucleotide linked via a linker that comprises an enzymatically cleavable linkage. The polymerase moiety of a conjugate can elongate a nucleic acid using its linked nucleotide (i.e., the polymerase can catalyze the attachment of a nucleotide to which it is joined onto a nucleic acid) and remains attached to the elongated nucleic acid via the linker until the linker is enzymatically cleaved.

In a conjugate, the linker comprises the atoms that connect the nucleotide to the polymerase. In some embodiments, the linker connects the base, the sugar, or the α-phosphate of a nucleotide to the polymerase. In some embodiments, the linker connects the terminal phosphate of a nucleotide to the polymerase. In some embodiments, the linker connects the nucleotide to the C_{α} atom in the backbone of the polymerase. In some embodiments, the polymerase and the nucleotide are covalently linked and the distance between the linked atom of the nucleotide and the polymerase to which it is attached may be in the range of 4-100 Å, e.g., 15-40 Å or 20-30 Å, although this distance may vary depending on where the nucleotide is tethered. The linker used should be sufficiently long to allow the nucleotide to access the active site of the polymerase to which it is tethered. As will be described in greater detail below, the polymerase of a conjugate is capable of catalyzing the addition of the nucleotide to which it is linked onto the 3′ end of a nucleic acid.

Linkers contemplated herein are also of sufficient length and stability to allow efficient hydrolysis by enzymatic means. The number of carbons or atom in a linker, optionally derivatized by other functional groups, must be of sufficient length to allow either enzymatic cleavage of the polymerase from the nucleotide.

In certain aspects, a cleavable linker comprises an amino acid ester. In some aspects, an amino acid ester is the site of cleavage of the linker, thereby facilitating release of a polymerase upon exposure to an esterase or protease comprising esterase activity. A portion of the cleavable linker comprising the amino acid ester is referred to herein as the “L²” portion of the linker. L² can be designed and optimized for enzymatic cleavage by an esterase or protease comprising esterase activity, for example, by modifying the chemical group attached to the alpha carbon of the amino acid ester, or by including one or more amino acids adjacent to the amino acid ester as part of L².

Described herein are polymerase-nucleotide conjugates comprising cleavable linkers that are highly stable and rapidly enzymatically cleavable by proteases comprising esterase activity. In some embodiments, a polymerase-nucleotide conjugate comprises a nucleotide linked to a polymerase using an enzymatically cleavable linker. In some embodiments, a polymerase-nucleotide conjugate comprising an enzymatically cleavable linker comprises a structure Nuc-L¹-L²-L³-Pol, wherein Nuc represents a nucleotide, pol represents a polymerase, and L¹-L²-L³ represents an enzymatically cleavable linker. In some embodiments, L¹ represents a region of an enzymatically cleavable linker connecting the nucleotide to L², L² represents a cleavable portion of an enzymatically cleavable linker, L³ represents a region of an enzymatically cleavable linker connecting L² to Pol.

In some embodiments, an enzymatically cleavable linker comprises an amino acid ester moiety. In some embodiments, L² comprises an amino acid ester moiety. In some embodiments, the ester group of an amino acid ester moiety is cleavable by a protease comprising esterase activity. The ester of an amino acid of L² is attached to L¹, which can also be referred to as a spacer or as a scar of a nucleotide after cleavage of the L² ester. L² is also attached to L³, the rest of the linker, which comprises attachment chemistry for polymerase conjugation. In some embodiments, L³ can also include or be referred to as a spacer. In some embodiments, L² further comprises additional amino acids bound to the amine of the amino acid ester to serve as a protease substrate. As described herein, L² is optimized for ester stability to prevent spontanous cleavage while retaining the ability to act as a suitable substrate for esterase activity of a protease comprising esterase activity.

In some embodiments, the linker is bound to the nucleotide at the nucleobase. In some embodiments, the linker is bound to the nucleotide at the sugar. In some embodiments, the linker is bound to the nucleotide at a 5′ phosphate group, wherein the nucleotide is any nucleoside polyphosphate. In some embodiments, the linker is bound to the alpha phosphate. In some embodiments, the linker is bound to the gamma, beta, delta, epsilon, zeta, eta, or theta phosphate. In some embodiments, the linker is bound to the terminal phosphate. In some embodiments, a linker of a conjugate may be attached to the 7-position of deaza dGTP or the 5-position of dTTP or dUTP.

Additional tethered nucleotides can be found, e.g., in PCT Publication WO2017/223517 “Nucleic Acid Synthesis and Sequencing Using Tethered Nucleoside Triphosphates,” the entirety of which is incorporated by reference.

In some embodiments, the tethered nucleotide may be specifically attached to a cysteine residue of the polymerase using a sulfhydryl-specific attachment chemistry. Possible sulfhydryl specific attachment chemistries include, but are not limited to ortho-pyridyl disulfide (OPSS), maleimide functionalities, 3-arylpropiolonitrile functionalities, allenamide functionalities, haloacetyl functionalities such as iodoacetyl or bromoacetyl, alkyl halides or perfluroaryl groups that can favorably react with sulfhydryls surrounded by a specific amino acid sequence (Zhang, Chi, et al. Nature chemistry 8, (2015) 120-128.). Other attachment chemistries for specific labeling of cysteine residues will be apparent to those skilled in the art or are described in the pertinent literature and texts (e.g., Kim, Younggyu, et al, Bioconjugate chemistry 19.3 (2008): 786-791.).

We have prepared and assayed several TdT-dNTP conjugates using different cleavable linkers to tether the nucleotide site-specifically to the polymerase. The use of a peptide bond in a linker results in a linker that can be cleaved by a protease. Cleavage of a peptide bond by a protease generates an amine and a carboxylic acid, both of which are charged under the buffer conditions that are typical for TdT activity. However, having charged functional groups persist on synthesized oligonucleotides can lead to deleterious effects during synthesis.

In contrast cleavage of an ester group generates an alcohol—a charge-neutral cleavage product. Herein, we demonstrate that nucleotides including scars containing such alcohols do not hinder conjugate-based oligonucleotide synthesis (see Example 2). We have also demonstrated that linkers including an amino acid ester can be cleaved enzymatically by a protease comprising esterase activity, such as Proteinase K (see Example 2).

Therefore, in some embodiments, L² comprises an amino acid ester. In some embodiments, the amino acid ester is the site of cleavage of the linker, facilitating the release of the polymerase from the nucleotide.

In addition, we initially observed that the ester group of a glycine amino acid ester in the linker could be unstable, resulting in spontaneous cleavage of the conjugate and unwanted nucleotide insertions during conjugate-based oligonucleotide synthesis (see Examples 2 and 3). However, addition of aliphatic or bulky substituents to the alpha carbon of the amino acid ester was observed to favorably improve stability of the adjacent ester (see Examples 4 and 6). In addition, substitution of atoms at the alpha carbon of the amino acid ester can affect hyperconjugation, resulting in an increase or decrease in the lability of the adjacent ester, as well as rate of cleavage by a protease comprising esterase activity. Thus, selection of a preferred substituent at the alpha carbon of the amino acid ester can be used to achieve an acceptable balance between stability and linker cleavage kinetics (see Example 6).

Therefore, in some embodiments, the amino acid ester comprises one or more substitutions at the alpha carbon, such as addition of an aliphatic or bulky substituent. In some embodiments, the amino acid ester is represented by:

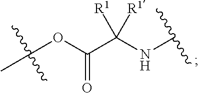

wherein R¹ and R^{1′} are each independently selected from an optionally substituted C₁₋₃ alkyl, a halogen, or are optionally taken together with the atom on which they are attached to form an optionally substituted C₃-C₇ carbocyclic ring.

Exemplary L² linker structures with different substituents on the alpha carbon of the amino acid ester (e.g., to improve stability of the ester) are shown below:

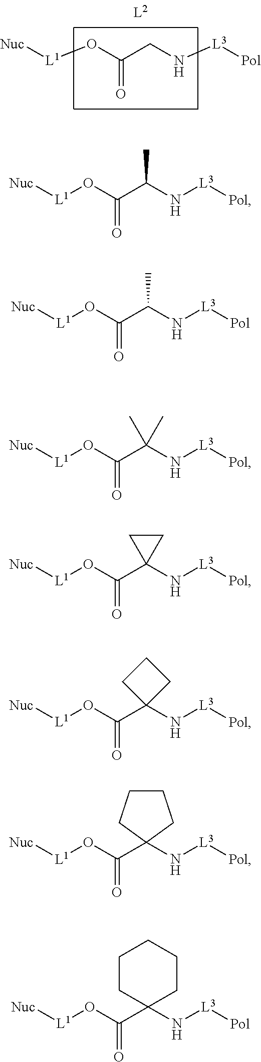

Furthermore, we observed that the kinetics of cleavage of the ester group by a protease comprising esterase activity could be improved by including one or more amino acids in the L² moiety adjacent to the amino acid ester (see Example 5). In some embodiments, L² comprises an amino acid ester adjacent to one or more amino acid residues. In some embodiments, the one or more amino acid residues are bound to the amine group of the amino acid ester.

In some embodiments, L² comprises or consists of:

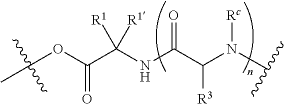

wherein
R¹ and R^{1′} are independently selected from hydrogen or an optionally substituted C₁₋₃ alkyl or are taken together with the atom on which they are attached to form an optionally substituted C₃-C₇ carbocyclic ring;
each R³ is an optionally substituted group independently selected from hydrogen, C₁₋₆ alkyl, benzyl, —OH, —O(C₁₋₆ alkyl), and —CN;
each R^{c} is hydrogen or optionally substituted C₁₋₆ alkyl; and
n is 1, 2, or 3.

In some embodiments, the one or more amino acids linked to the amine of the amino acid ester comprise L- or D-isomers of amino acid residues. The term “naturally-occurring amino acid” refer to Ala, Asp, Cys, Glu, Phe, Gly, His, He, Lys, Leu, Met, Asn, Pro, Gin, Arg, Ser, Thr, Val, Trp, Tyr, or citrulline. “D-” designates an amino acid having the “D” (dextrorotary) configuration, as opposed to the configuration in the naturally occurring (“L-”) amino acids. The amino acids described herein can be purchased commercially (Sigma Chemical Co., Advanced Chemtech) or synthesized using methods known in the art. In some embodiments, amino acids with non-natural or artificial side chains are linked to the amine of the amino acid ester.

As discussed above, we observed that the composition of the linker (i.e. the peptide sequence of L²) has a significant impact on the rate of protease-mediated deprotection. Accordingly, various permutations of amino acids in L² could yield conjugates with faster addition and deprotection kinetics. Such linkers could include variations of amino acid identity and number of consecutive amino acids.

The one or more amino acids included in the L² portion of the linker/bound to the amino acid ester can be selected, for example, to optimize protease binding and ester cleavage. A combinatorial library can be generated to test optimal cleavage activity, amino acids can be chosen based on existing known peptide sequence targets for the protease. The protease comprising esterase activity can recognize the peptide portion of the linker and hydrolyzes the ester group of the amino acid ester of L², resulting in removal of polymerase attached to the nucleotide via the linker, as disclosed herein.

If desired, a spacer can be used between the nucleotide and the linker, or between the linker and the label. Different lengths of spacers can be used in order to increase L2 availability towards the protease/esterase and increase the efficiency and fidelity of polymerases. Exemplary spacers include, for example, polyethyleneglycol or other suitable spacers.

Examples of linkers comprising L² structures including an amino acid ester bound to one or more amino acid residues is shown below:

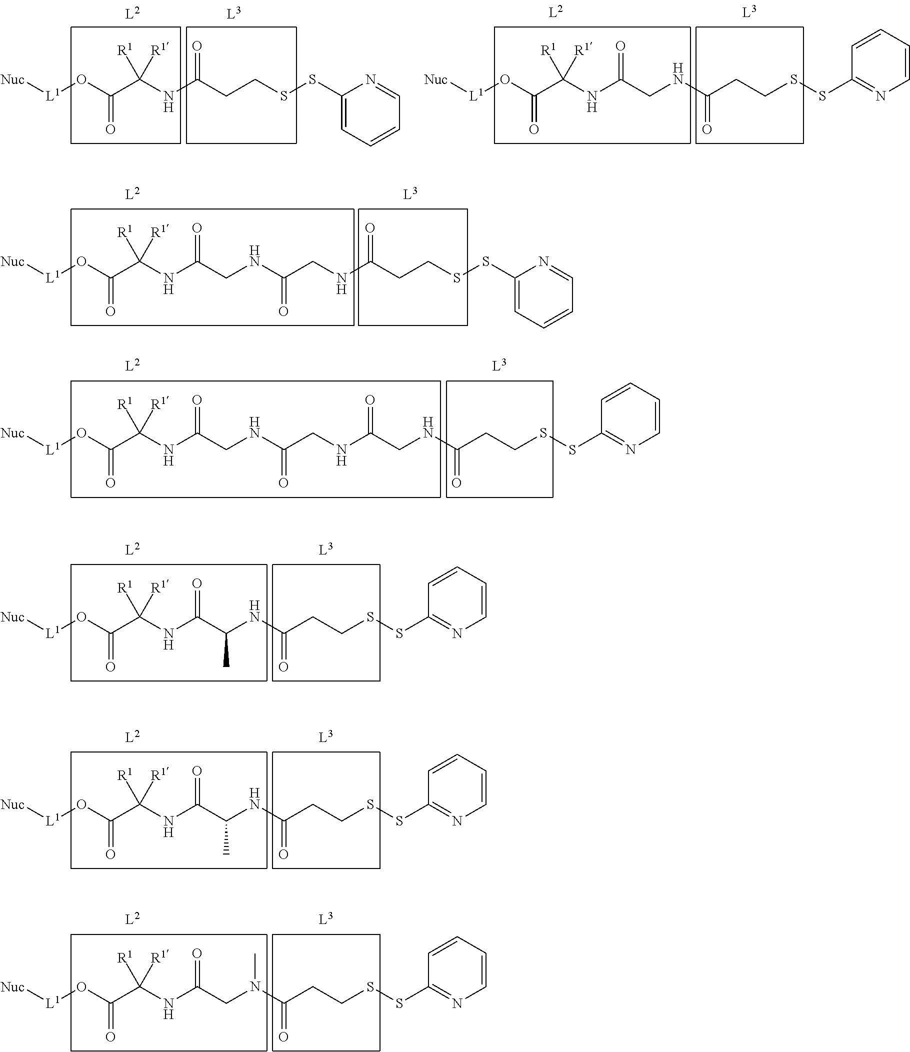

There is considerable flexibility on the type of linker used for regions of the linker not associated with enzymatic cleavage disclosed herein (i.e., L¹ and L³). Examples of suitable linker structures may include, but are not limited to, carbon-chain linkers (e.g., C6, C12, C18, C24, etc.), peptide linkers (e.g., poly-glycine or poly-alanine ranging from about 1 residue to about 1,000 residues in length), or polyether linkers (e.g., PEG, PPG, PAG, PTMG from about 1 polyether unit to about 1,000 polyether units in length).

In some embodiments, L¹ or L³ is a chain of atoms selected from C, N, O, S, Si, and P, preferably having 0-500 atoms, wherein L¹ covalently connects to Nuc and L², and wherein L³ covalently connects to L² and Pol. The atoms used in forming L¹ or L3 may be combined in all chemically relevant ways, such as forming alkylene, alkenylene, and alkynylene, carbamates, carbonates, ethers, polyoxyalkylene, esters, amines, imines, polyamines, hydrazines, hydrazones, amides, ureas, semicarbazides, carbazides, alkoxyamines, alkoxylamines, urethanes, amino acids, peptides, acyloxylamines, hydroxamic acids, or combination above thereof.

In some embodiments, L¹ or L³ comprises one or more carbon atoms, zero, one, or more oxygen atoms, zero, one or more nitrogen atoms, zero, one, or more sulfur atoms, or a combination thereof, in different embodiments. In some embodiments, L¹ or L³ comprise, comprise about, comprise at least, comprise at least about, comprise at most, or comprise at most about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 2000, 3000, 4000, 5000, 6000, 7000, 8000, 9000, 10000, or a number or a range between any two of these values, carbon atom(s), oxygen atom(s), nitrogen atom(s), sulfur atom(s), or a combination thereof.

In some embodiments, L¹ or L³ comprise a polymer, such as a homopolymer or a heteropolymer. In some embodiments, L¹ or L³ comprise a plurality of repeat units. In some embodiments, the plurality of repeating units comprises identical repeating units. In some embodiments, the plurality of repeating units comprises two or more different repeating units. The plurality of repeating units can comprise a polyether such as paraformaldehyde, polyethylene glycol (PEG), polypropylene glycol (PPG), polyalkylene glycol (PAG), polytetramethylene glycol (PTMG), or a combination thereof. For example, the plurality of repeating units can comprise PEGix, PEG23, PEG24, or a combination thereof. The plurality of repeating units can comprise a polyalkylene, such as polyethene, polypropene, polybutene, or a combination thereof. In some embodiments, a repeating unit of the plurality of repeating units comprises no aromatic group. In some embodiments, a repeating unit of the plurality of repeating units comprises one or more aromatic groups.

In some embodiments, L¹ or L³ comprises any number of basic chemical starting blocks. For example, linkers may comprise linear or branched alkyl, alkenyl, or alkynyl chains, or combinations thereof, that provide a useful distance between the nucleotide and polymerase, the nucleotide and L², or the polymerase and L². For instance, amino-alkyl linkers, e.g., amino-hexyl linkers, have been used to attach linkers to nucleotide analogs, and are generally sufficiently rigid to maintain such distances. The longest chain of such linkers may include as many as 2 atoms, 3 atoms, 4 atoms, 5 atoms, 6 atoms, 7 atoms, 8 atoms, 9 atoms, 10 atoms, or even 11-35 atoms, or even 35-50 atoms. The linear or branched linker may also contain heteroatoms other than carbon, including, but not limited to, oxygen, sulfur, phosphate, and nitrogen. A polyoxyethylene chain (also commonly referred to as polyethyleneglycol, or PEG) is a preferred linker constituent due to the hydrophilic properties associated with polyoxyethylene. Insertion of heteroatom such as nitrogen and oxygen into the linkers may affect the solubility and stability of the linkers.

The linker, including L¹ or L³, may be rigid in nature or flexible. Rigid structures include laterally rigid chemical groups, e.g., ring structures such as aromatic compounds, multiple chemical bonds between adjacent groups, e.g., double or triple bonds, in order to prevent rotation of groups relative to each other, and the consequent flexibility that imparts to the overall linker. Thus, the degree of desired rigidity may be modified depending on the content of the linker, or the number of bonds between the individual atoms comprising the linker. Further, addition of ringed structures along the linker may impart rigidity. Ringed structures may include aromatic or non-aromatic rings. Rings may be anywhere from 3 carbons, to 4 carbons, to 5 carbons or even 6 carbons in size. Rings may also optionally include heteroatoms such as oxygen or nitrogen and also be aromatic or non-aromatic. Rings may additionally optionally be substituted by other alkyl groups and/or substituted alkyl groups.

Linkers that comprise ring or aromatic structures can include, for example aryl alkynes and aryl amides. Other examples of the linkers of the disclosure include oligopeptide linkers that also may optionally include ring structures within their structure.

In embodiments, L¹ or L³ is a C₁-C₁₀ alkylene chain, wherein 1-6 methylene units are optionally and independently replaced by —NH—, —O—, —C(O)—, —C(O)NH—, —NHC(O)—, —NHC(O)NH—, —C(O)O—, —OC(O)—, —SS—, optionally substituted cycloalkylene (e.g., C₃-C₈, C₃-C₆, or C₅-C₆), optionally substituted heterocycloalkylene (e.g., 3 to 8, 3 to 6, or 5 to 6 membered), optionally substituted arylene (e.g., C₆-C₁₀, C₁₀, or phenylene), or substituted or unsubstituted heteroarylene (e.g., 5 to 10, 5 to 9, or 5 to 6 membered).

In embodiments, L¹ or L³ is a bond, —NH—, —O—, —C(O)—, —C(O)NH—, —NHC(O)—, —NHC(O)NH—, —C(O)O—, —OC(O)—, —SS—, optionally substituted alkylene (e.g., C₁-C₂₀, C₁₀-C₂₀, C₁-C₈, C₁-C₆, or C₁-C₄), optionally substituted heteroalkylene (e.g., 2 to 20, 8 to 20, 2 to 10, 2 to 8, 2 to 6, or 2 to 4 membered), optionally substituted cycloalkylene (e.g., C₃-C₈, C₃-C₆, or C₅-C₆), optionally substituted heterocycloalkylene (e.g., 3 to 8, 3 to 6, or 5 to 6 membered), optionally substituted (e.g., C₆-C₁₀, C₁₀, or phenylene), or optionally substituted (e.g., 5 to 10, 5 to 9, or 5 to 6 membered). In embodiments, L¹ or L³ is optionally substituted C₁-C₂₀ alkylene. In some embodiments, L¹ or L³ optionally substituted 2 to 20 membered heteroalkylene. In embodiments, L¹ or L³ is optionally substituted C₃-C₈ cycloalkylene. In some embodiments, L¹ or L³ is optionally substituted 3 to 8 membered heterocycloalkylene. In embodiments, L¹ or L³ is optionally substituted C₆-C₁₀ arylene. In embodiments, L¹ or L³ is optionally substituted 5 to 10 membered heteroarylene.

In some embodiments, L¹ is substituted with 1-6 instances of R^{L}. In some embodiments, L³ is substituted with 1-6 instances of R^{L}. Each R^{L} is independently selected from the group consisting of oxo, halogen, —CCI₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₂, —OCHCI₂, —OCHBr₂, —OCHb, —OCHF₂, —N₃, optionally substituted alkyl (e.g., C₁-C₂₀, C₁₀-C₂₀, C₁-C₈, C₁-C₆, or C₁-C₄), optionally substituted heteroalkyl (e.g., 2 to 20, 8 to 20, 2 to 10, 2 to 8, 2 to 6, or 2 to 4 membered), optionally substituted cycloalkyl (e.g., C₃-C₈, C₃-C₆, or C₅-C₆), optionally substituted heterocycloalkyl (e.g., 3 to 8, 3 to 6, or 5 to 6 membered), optionally substituted aryl (e.g., C₆-C₁₀, C₁₀, or phenyl), and optionally substituted heteroaryl (e.g., 5 to 10, 5 to 9, or 5 to 6 membered).

In embodiments, L¹ or L³ is —(CH₂CH₂O)_{b}—. In embodiments, L¹ or L³ is —CCCH₂(OCH₂CH₂)ₐ—NHC(O)—(CH₂)_{c}(OCH₂CH₂)_{b}—. In embodiments, L¹ or L³ is —CHCHCH₂—NHC(O)—(CH₂)_{c}(OCH₂CH₂)_{b}—. In embodiments, L¹ or L³ is —CCCH₂—NHC(O)—(CH₂)_{c}(OCH₂CH₂)_{b}—. In embodiments, L¹ or L³ is —CCCH₂—. The symbol a is an integer from 0 to 8. In embodiments, a is 1. In embodiments, a is 0. The symbol b is an integer from 0 to 8. In embodiments, b is 1 or 2. In embodiments, b is an integer from 2 to 8. In embodiments, b is 1. The symbol c is an integer from 0 to 8. In embodiments, c is 3. In embodiments, c is 1. In embodiments, c is 2. In embodiments, L1 or L3 is independently a substituted or unsubstituted C₁-C₄ alkylene or substituted or unsubstituted 8 to 20 membered heteroalkylene.

L1 acts as an attachment point to the nucleotide and includes a hydroxyl terminal group which binds to a portion of L2 during synthesis.

In some embodiments, L1 is a scar that is enzymatically or chemically cleavable after cleavage of polymerase-nucleotide linker/removal of the L2-L3-pol moiety.

In some embodiments, L1 is selected from the group consisting of a bond, an optionally substituted C₁₋₁₂ alkylene chain, C₄-C₂₀ polyethylene glycol, an optionally substituted C₂₋₁₂ alkenylene chain, and an optionally substituted C₂₋₁₂ alkynylene chain, wherein 1-4 methylene units of L¹ are optionally and independently replaced with —O—, —N(R^{b})—, —C(O)—, —S—, —S(O)—, —S(O)₂—, phenylene, cyclopropylene; wherein each R^{b} is independently hydrogen or optionally substituted C₁₋₆ alkyl.

In some embodiments, L1 comprises:

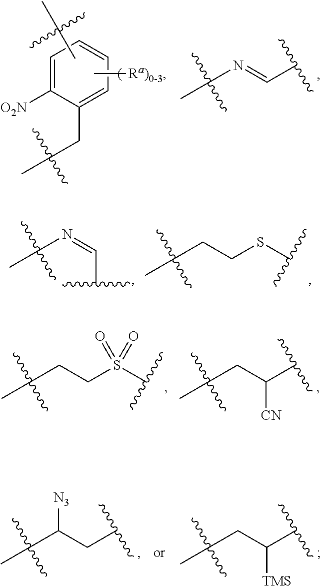

wherein each R^{a} is independently selected from the group consisting of halogen, hydroxyl, cyano, optionally substituted C₁₋₆ alkyl, and optionally substituted C₁₋₆ alkoxy.

In some embodiments, L¹ is selected from the group consisting of:

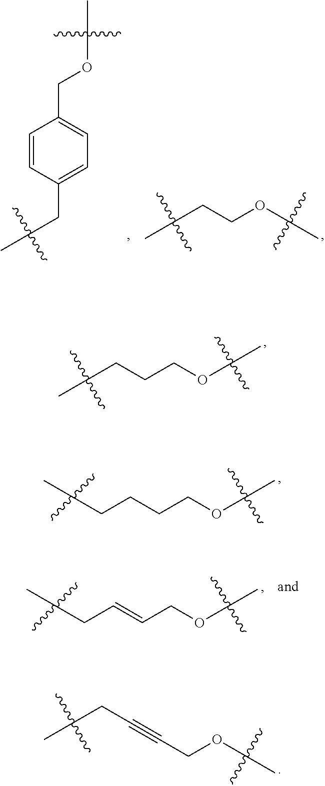 [SMILES: *Cc1ccc(CO*)cc1 ; *CCO* ; *CCCO* ; *CCCCO* ; *C/C=C/CO* ; *CC#CCO*]

In some embodiments, L3 comprises a bioconjugate group suitable for conjugation of L3 to the polymerase.

In some embodiments, the bioconjugate group is an N-hydroxysuccinimide ester (NHS) group. In some embodiments, the bioconjugate group is a maleimide group. The linker may then be covalently attached to the polymerase by reaction of the maleimide group with a cysteine residue of the polymerase.

In some embodiments, the polymerase may be operably linked to a linker moiety including a covalent or non-covalent bond; amino acid tag (e.g., poly-amino acid tag, poly-His tag, 6His-tag); chemical compound (e.g., polyethylene glycol); protein-protein binding pair (e.g., biotin-avidin); affinity coupling; capture probes; or any combination of these. The linker moiety can be separate from or part of a polymerase variant.

In some embodiments, the linker connecting the nucleotide and the polymerase comprises a saturated or unsaturated, substituted or unsubstituted, straight or branched carbon chain. The length of the linker can be different in different embodiments. The length of the linker may vary depending on the type of nucleotide and the polymerase. In some embodiments, the linker length in the enzyme linked nucleotide is different for each different nucleotide or nucleotide analog. In some embodiments, the linker has a length of, of about, of at least, of at least about, of at most, or of at most about, 19 Å, 20 Å, 21 Å, 22 Å, 23 Å, 24 Å, 25 Å, 26 Å, 27 Å, 28 Å, 29 Å, 30 Å, 31 Å, 32 Å, 33 Å, 34 Å, 35 Å, 36 Å, 37 Å, 38 Å, 39 Å, 40 Å, 41 Å, 42 Å, 43 Å, 44 Å, 45 Å, 46 Å, 47 Å, 48 Å, 49 Å, 50 Å, 51 Å, 52 Å, 53 Å, 54 Å, 55 Å, 56 Å, 57 Å, 58 Å, 59 Å, 60 Å, 61 Å, 62 Å, 63 Å, 64 Å, 65 Å, 66 Å, 67 Å, 68 Å, 69 Å, 70 Å, 71 Å, 72 Å, 73 Å, 74 Å, 75 Å, 76 Å, 77 Å, 78 Å, 79 Å, 80 Å, 81 Å, 82 Å, 83 Å, 84 Å, 85 Å, 86 Å, 87 Å, 88 Å, 89 Å, 90 Å, 91 Å, 92 Å, 93 Å, 94 Å, 95 Å, 96 Å, 97 Å, 98 Å, 99 Å, 100 Å, 200 Å, 300 Å, 400 Å, 500 Å, 600 Å, 700 Å, 800 Å, 900 Å, 1000 Å, or a number or a range between any two of these values. In some embodiments, the polymerase and the nucleotide are covalently linked, and the distance between the linked atom of the nucleotide and the polymerase is from about 4 Å to about 100 Å. In some embodiments, the distance between the linked atom of the nucleotide and the polymerase is about 5 Å to about 20 Å. In some embodiments, the distance between the linked atom of the nucleotide and the polymerase is about 20 Å to about 50 Å. In some embodiments, the distance between the linked atom of the nucleotide and the polymerase is about 50 Å to about 75 Å. In some embodiments, the distance between the linked atom of the nucleotide and the polymerase is about 75 Å to about 100 Å.

In some embodiments, the length of the linker will be defined as its persistence length, corresponding to the root-mean-square (RMS) distance between the ends of the linker as characterized by dynamic simulations, 2-D trapping experiments, or ab initio calculations based on statistical distributions of polymers in compact, collapsed, or fluid states as required by the solution, suspension, or fluid conditions present. In some embodiments, a linker may have a persistence length of at least 0.1, at least 0.2, at least 0.4, at least 1, at least 2, at least 4, at least 10, at least 20, at least 30, at least 40, at least 50, at least 60, at least 80, at least 90, at least 100, at least 200, at least 300, at least 400, at least 500, at least 700, or at least 1,000 nm, or a persistence length in a range defined by or comprising any two or more of these values. In some embodiments, a linker for connecting the nucleotide to the enzyme can have a persistence length of about 0.1-1,000 nm, 0.5-500 nm, 0.5-400 nm, 0.5-300 nm, 0.5-200 nm, 0.5-100 nm, 0.5-50 nm, 1-500 nm, 1-400 nm, 1-300 nm, 1-200 nm, 1-100 nm, 1-50 nm, 1.5-500 nm, 1.5-400 nm, 1.5-300 nm, 1.5-200 nm, 1.5-100 nm, 1.5-50 nm, 5-500 nm, 5-400 nm, 5-300 nm, 5-200 nm, 5-100 nm, or 5-50 nm. In some embodiments, the linker may have a persistence length of shorter than about 5, 10, 20, 30, 40, 50, 60, 80, 100, 200, 300, 400, 500, 700, or 1,000 nm. In some embodiments, linkers provided for one nucleotide may be longer or shorter than the linker provided for another nucleotide. In some embodiments, linkers provided for one polymerase may be longer or shorter than the linker provided for another polymerase.

### Conjugate

In some embodiments, the conjugate is represented by

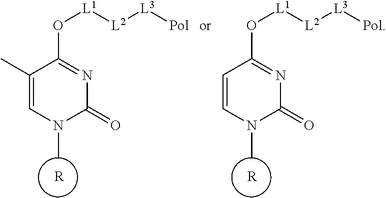

In some embodiments, a conjugate is represented by a structure of Formula (I) or (II):

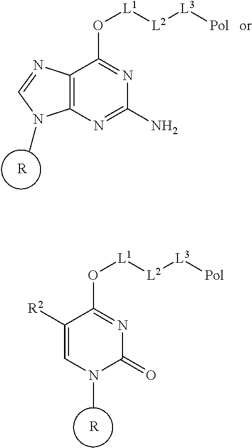

wherein
L¹ is selected from the group consisting of an optionally substituted C₁₋₆ alkylene chain, an optionally substituted C₂₋₆ alkenylene chain, and an optionally substituted C₁₋₆ alkynylene chain, wherein 1-4 methylene units are optionally and independently replaced with —O—, —N(R^{a})—, —C(O)—, —S—, —S(O)—, —S(O)₂—, or phenylene;
L² is a cleavable linker;
L³ is a linker connecting pol to L²
each R^{a} is independently hydrogen or C₁₋₆ alkyl;
R² is hydrogen or methyl;
R is a ribose polyphosphate or deoxyribose polyphosphate; and
Pol is a polymerase.

In some embodiments, the conjugate is selected from the group consisting of

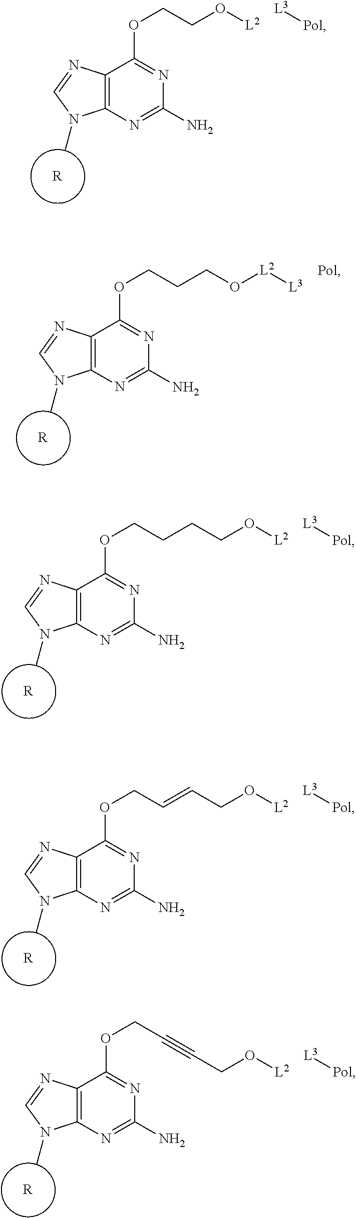

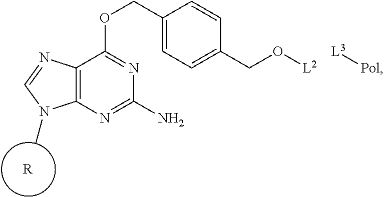

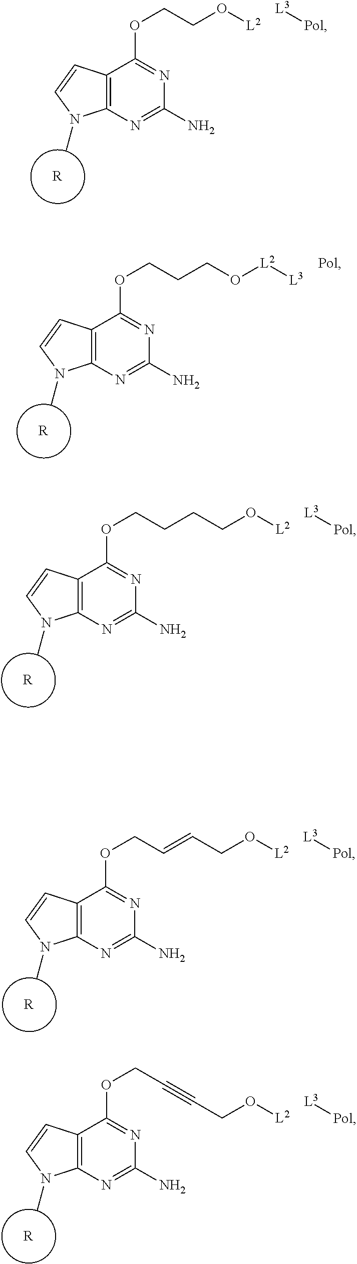

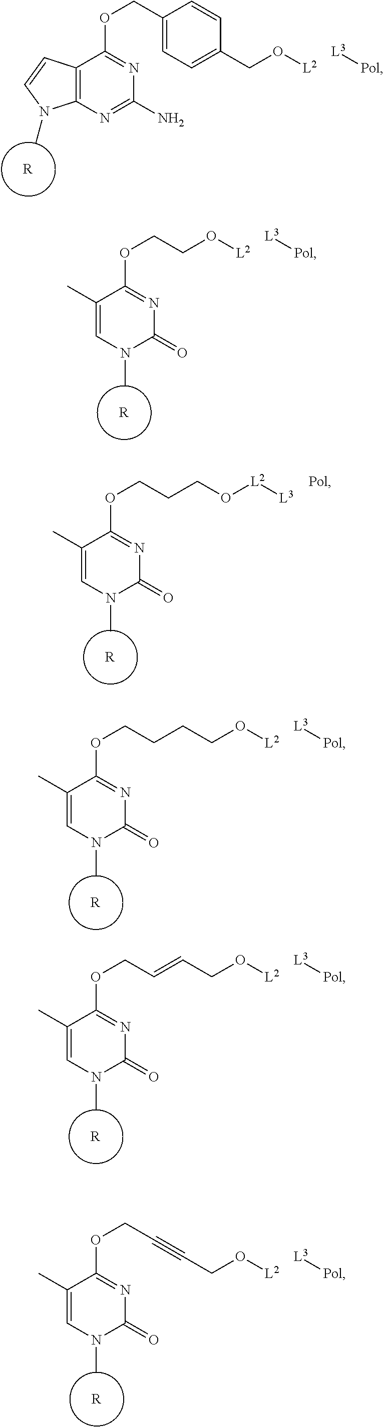

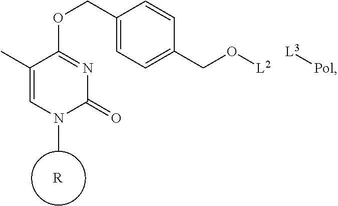

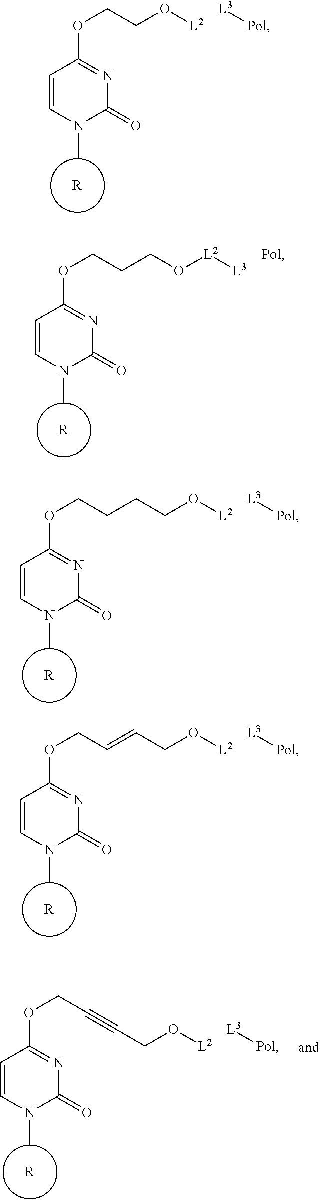

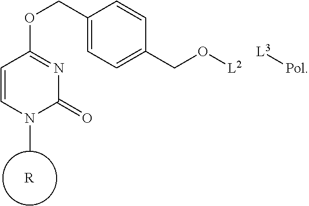

When a conjugate comprising a polymerase and a nucleotide is incubated with a nucleic acid, it preferentially elongates the nucleic acid using its tethered nucleotide (as opposed to using the nucleotide of another conjugate molecule). As described above, the polymerase then remains attached to the nucleic acid via its tether to the added nucleotide until exposed to some stimulus that causes cleavage of the linkage to the added nucleotide. In this situation, further extensions by polymerase-nucleotide conjugates are hindered due to “shielding” when: 1) the attached polymerase molecule hinders other conjugates from accessing the 3′ OH of the extended DNA molecule and 2), other nucleotides in the system are hindered from accessing the catalytic site of the polymerase that remains attached to the 3′ end of the extended nucleic acid. (The extent of shielding may be described as the extent to which both of these interactions are hindered.) To enable subsequent extensions, the linker tethering the incorporated nucleotide to the polymerase can be cleaved, releasing the polymerase from the nucleic acid and therefore re-exposing its 3′ OH group for subsequent elongation.

Methods for nucleic acid synthesis provided herein that employ the shielding effect to achieve termination comprise an extension step wherein a nucleic acid is exposed to conjugates preferentially in the absence of free (i.e. untethered) nucleoside triphosphates, because the termination mechanism of shielding may not prevent their incorporation into the nucleic acid.

In some embodiments, termination of further elongation may be “complete”, meaning that after a nucleic acid molecule has been elongated by a conjugate, further elongations cannot occur during the reaction. In other embodiments, termination of further elongation may be “incomplete”, meaning that further elongations can occur during the reaction but at a substantially decreased rate compared to the initial elongation, e.g., 100 times slower, or 1000 times slower, or 10,000 times slower, or more. Conjugates that achieve incomplete termination may still be used to extend a nucleic acid by predominantly a single nucleotide (e.g. in methods for nucleic acid synthesis and sequencing) when the reaction is stopped after an appropriate amount of time. In some embodiments, the reagent containing the conjugate may additionally contain polymerases without tethered nucleotides, but those polymerases should not significantly affect the reaction because there are no free dNTPs in the mix.

Reagents based on conjugates employing the shielding effect to achieve termination preferentially only contain polymerase-nucleotide conjugates in which all polymerases remain folded in the active conformation. In some cases, if the polymerase moiety of a conjugate is unfolded, its tethered nucleotide may become more accessible to the polymerase moieties of other conjugate molecules. In these cases, the unshielded nucleotides may be more readily incorporated by other conjugate molecules, circumventing the termination mechanism.

Polymerase-nucleotide conjugates employing the shielding effect to achieve termination are preferentially only labeled with a single nucleotide moiety. Polymerase-nucleotide conjugates labeled with multiple nucleotides that can access the catalytic site can, in some cases, incorporate multiple nucleotides into the same nucleic acid. Additional tethered nucleotides may therefore lead to additional, undesired nucleotide incorporations into a nucleic acid during a reaction. Furthermore, only one tethered nucleotide can occupy the (buried) catalytic site of its polymerase at a time so the other tethered nucleotide(s) may have an increasing accessibility to the polymerase moieties of other conjugate molecules, as discussed below.

Polymerase-nucleotide conjugates employing the shielding effect to achieve termination preferentially comprise as short of a linker as possible that still enables the nucleotide to frequently access the catalytic site of its tethered polymerase molecule in a productive conformation, in order to enable fast incorporation of the nucleotide into a nucleic acid. Such conjugates may also preferentially employ an attachment position of the linker to the polymerase as close to the catalytic site as possible, enabling use of a shorter linker. The length of the linker will determine the maximum distance from the attachment point a tethered nucleotide or a tethered nucleic acid can reach. A smaller distance may lead to a reduced accessibility of the tethered moiety to other polymerase-nucleotide molecules, as discussed below. In some embodiments, linkers are approximately 24 and 28 Å long. Shorter linkers, e.g. with lengths of 8-15 Å may increase shielding; longer linkers, e.g. linkers longer than 50 Å, 70 Å or 100 Å, may reduce shielding. The shielding effect may be influenced by a combination of factors including, but not limited to, to the structure of the polymerase, the length of the linker, the structure of the linker, the attachment position of the linker to the polymerase, the binding affinity of the nucleotide to the catalytic site of the polymerase, the binding affinity of the nucleic acid to the polymerase, the preferred conformation of the polymerase, and the preferred conformation of the linker.

One contribution to shielding can be steric effects that block the 3′ OH of a nucleic acid that has been elongated by a conjugate from reaching into the catalytic site of another conjugate's polymerase moiety. Steric effects may also hinder a tethered nucleotide from reaching into the catalytic site of another polymerase-nucleotide conjugate molecule due to clashes between the conjugates that would occur during such approaches. These steric effects may result in complete termination if they completely block productive interactions between the tethered nucleotide (or elongated nucleic acid) of one conjugate molecule with another conjugate molecule, or may result in incomplete termination if they only hinder such intermolecular interactions.

Another contribution to shielding arises from the binding affinity of the tethered nucleotide to the catalytic site of the polymerase. The tethered nucleotide of a conjugate will have a high effective concentration with respect to the catalytic site of its tethered polymerase so it may remain bound to that site much of the time. When the nucleotide is bound to the catalytic site of its tethered polymerase molecule it is unavailable for incorporation by other polymerase molecules. Thus, tethering reduces the effective concentration of nucleotide available for intermolecular incorporation (i.e. incorporation catalyzed by a polymerase molecule to which the nucleotide is not tethered). This shielding effect can enhance termination by reducing the rate by which a nucleic acid is elongated using the nucleotide moiety of one conjugate molecule by the polymerase moiety of another conjugate molecule.

Another contribution to shielding arises from the binding affinity of the 3′ region of a nucleic acid molecule to the catalytic site of a polymerase molecule. After elongation by a conjugate, the nucleic acid is tethered to the conjugate via it's 3′ terminal nucleotide and will have a high effective concentration with respect to the catalytic site of its tethered polymerase so it may remain bound to that site much of the time. When the nucleic acid is bound to the catalytic site of its tethered polymerase molecule it is unavailable for elongation by other conjugate molecules. This effect can enhance termination by reducing the rate by which a nucleic acid that has been elongated by a first conjugate is further elongated by other conjugate molecules.

In some embodiments, the polymerase-nucleotide conjugates comprise additional moieties that sterically hinder the tethered nucleotide (or a tethered nucleic acid post-elongation) from approaching the catalytic sites of another conjugate molecule. Such moieties include polypeptides or protein domains that can be inserted into a loop of the polymerase, and those and other bulky molecules such as polymers that can be site-specifically ligated e.g. to an inserted unnatural amino acid or specific polypeptide tag.

Tethered nucleotides can have a high effective concentration, enabling fast incorporation kinetics. A tethered nucleotide will have a certain occupancy rate with the active site of the polymerase depending on the length and geometry of the linker and its attachment site on the protein. This rate can be expressed as an effective concentration (the concentration of free nucleotide that would give an equivalent occupancy rate). By varying the linker properties and attachment site, it is possible to control the effective concentration of the nucleotide, enabling high effective concentrations and therefore fast incorporations. For example, a very rough calculation suggests that the effective concentration of a dNTP tethered by a 20 Å linker will be ˜50 mM, (one molecule in the volume of a sphere with 20 Å radius). In this example, one could increase the local concentration of the dNTP by shortening the linker, or decrease it by lengthening the linker. In addition, having the nucleotide tethered inside the polymerase increases the effective concentration of the nucleotide because the full sphere cannot be accessed due to restricting interaction with the polymerase. For example, if the nucleotide was tethered to a perfectly flat surface, it could only occupy half of that sphere, and so the effective concentration within that half sphere would double relative to the untethered nucleotide

### Polymerase

Any polymerase capable of extending a polynucleotide, incorporating a nucleotide into a polynucleotide, or incorporating a nucleotide analog into a polynucleotide is envisaged for use in the conjugates and methods described herein. In some embodiments, the polynucleotide is single stranded. In some embodiments, the polynucleotide is double stranded. In some embodiments, the polynucleotide is immobilized on a solid support.

Examples of DNA polymerases include polA, polB, polC, polD, polY, polX, reverse transcriptases (RT), and high-fidelity polymerases. In some instances, the polymerase is a modified polymerase. In some embodiments, the polymerase comprises 29, B103, GA-1, PZA, 15, BS32, M2Y, Nf, G1, Cp-1, PRD1, PZE, SF5, Cp-5, Cp-7, PR4, PR5, PR722, L17, ThermoSequenase®, 9° Nm™, Therminator™ DNA polymerase, Tne, Tma, Tfl, Tth, TIi, Stoffel fragment, Vent™ and Deep Vent™ DNA polymerase, KOD DNA polymerase, Tgo, JDF-3, Pfu, Taq, T7 DNA polymerase, T7 RNA polymerase, PGB-D, UlTma DNA polymerase, E. coli DNA polymerase I, E. coli DNA polymerase III, archaeal DP1I/DP2 DNA polymerase II, 9° N DNA Polymerase, Taq DNA polymerase, Phusion® DNA polymerase, Pfu DNA polymerase, SP6 RNA polymerase, RB69 DNA polymerase, Avian Myeloblastosis Virus (AMV) reverse transcriptase, Moloney Murine Leukemia Virus (MMLV) reverse transcriptase, SuperScript® II reverse transcriptase, and SuperScript® III reverse transcriptase.

In some embodiments, the polymerase is DNA polymerase 1-KI enow fragment, Vent polymerase, Phusion® DNA polymerase, KOD DNA polymerase, Taq polymerase, T7 DNA polymerase, T7 RNA polymerase, Therminator™ DNA polymerase, POLB polymerase, SP6 RNA polymerase, E. coli DNA polymerase I, E. coli DNA polymerase III, Avian Myeloblastosis Virus (AMV) reverse transcriptase, Moloney Murine Leukemia Virus (MMLV) reverse transcriptase, SuperScript® II reverse transcriptase, or SuperScript® III reverse transcriptase.

The polymerase molecules used in the methods described herein can be polymerase theta, a DNA polymerase, or any enzyme that can extend nucleotide chains. In some embodiments, the polymerase is tri29. In some embodiments, the polymerase is a protein with pockets that work around terminal phosphate groups, for example, a triphosphate group. In some embodiments, the described methods use TdT with 1, 2, 3, 4, 5, 6, 7, 8, 9, or 10 amino acid mutations to synthesize defined polynucleotides. In some embodiments, the described method uses TdT with 1, 2, 3, 4, 5, 6, 7, 8, 9, or 10 amino acid mutations to a surface-accessible amino acid residue. In some embodiments, the TdT is a variant of TdT. In some embodiments, the variant of TdT comprises a cysteine mutation. In some embodiments, the polymerase is mutated to improve addition of a modified nucleotide bound to the polymerase forming a conjugate. In some instances, the variant TdT comprises at least 70%, 80%, 90%, or 95% sequence identity to wild-type TdT.

In some embodiments, the described methods use polymerase theta with 1, 2, 3, 4, 5, 6, 7, 8, 9, or 10 amino acid mutations to synthesize defined polynucleotides. In some embodiments, the described method uses polymerase theta with 1, 2, 3, 4, 5, 6, 7, 8, 9, or 10 amino acid mutations to a surface-accessible amino acid residue. In some embodiments, the polymerase theta is a variant of polymerase theta. In some instances, the variant polymerase theta comprises at least 70%, 80%, 90%, or 95% sequence identity to wild-type polymerase theta. In some embodiments, the polymerase theta is encoded by POLQ.

Enzymes described herein (e.g., TdT), in some embodiments, comprise one or more unnatural amino acids. In some instances, the unnatural amino acid comprises: a lysine analogue; an aromatic side chain; an azido group; an alkyne group; or an aldehyde or ketone group. In some instances, the unnatural amino acid does not comprise an aromatic side chain. In some embodiments, the unnatural amino acid is selected from N6-azidoethoxy-carbonyl-L-lysine (AzK), N6-propargylethoxy-carbonyl-L-lysine (PraK), N6-(propargyloxy)-carbonyl-L-lysine (PrK), p-azido-phenylalanine, BCN-L-lysine, norbornene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenyl alanine, p-benzoyl-L-phenylalanine, p-bromophenylalanine, p-amino-L-phenyl alanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, selenocysteine, N6-(((2-azidobenzyl)oxy)carbonyl)-L-lysine, N6-(((3-azidobenzyl)oxy)carbonyl)-L-lysine, and N6-(((4-azidobenzyl)oxy)carbonyl)-L-lysine.

In some embodiments, the polymerase is a fusion protein. In some embodiments of the method, the fusion protein comprises maltose binding protein (MBP). In some embodiments, TdT is fused to other enzymes such as helicase.

In some embodiments, the polymerase comprises a template-independent polymerase. In some embodiments, the polymerase comprises a Pol-X family polymerase. In some embodiments, the polymerase comprises a Terminal deoxynucleotidyl Transferase (TdT), or a variant thereof. In some embodiments, the template-independent polymerase comprises a TdT or a variant thereof. In some embodiments, the TdT or variant thereof comprises a sequence sharing at least 60%, at least 65%, at least 70%, at least 75%, at least 80%, at least 85%, at least 90%, at least 95%, at least 96%, at least 97%, at least 98%, or at least 99% identity to SEQ ID NO: 1. In some embodiments of the method, the TdT comprises a sequence identical to SEQ ID NO: 1. In some embodiments, the TdT variant comprises one or more amino acid substitutions, insertions, or deletions to SEQ ID NO: 1.

>Terminal deoxynucleotidyl transferase (TdT)
(SEQ ID NO: 1)
MGGRDIVDGSEFSPSPVPGSQNVPAPAVKKISQYACQRRTTLNNYNQLF
TDALDILAENDELRENEGSALAFMRASSVLKSLPFPITSMKDTEGIPSL
GDKVKSIIEGIIEDGESSEAKAVLNDERYKSFKLFTSVFGVGLKTAEKW
FRMGFRTLSKIQSDKSLRFTQMQKAGFLYYEDLVSCVNRPEAEAVSMLV
KEAVVTFLPDALVTMTGGFRRGKMTGHDVDFLITSPEATEDEEQQLLHK
VTDFWKQQGLLLYADILESTFEKFKQPSRKVDALDHFQKCFLILKLDHG
RVHSEKSGQQEGKGWKAIRVDLVMSPYDRRAFALLGWTGSRQFERDLRR
YATHERKMMLDNHALYDRTKRVFLEAESEEEIFAHLGLDYIEPWERNA

In some embodiments, template-independent polymerases having activity as described for E.C. class 2.7.7.31 are used. In some embodiments, the template-indepependent polymerase is a deoxynucleotidyl transferase or DNA nucleotidylexotransferase. A description of such enzymes can be found in Bollum, F. J. Deoxynucleotide-polymerizing enzymes of calf thymus gland. V. Homogeneous terminal deoxynucleotidyl transferase. J. Biol. Chem. 246 (1971) 909-916; Gottesman, M. E. and Canellakis, E. S. The terminal nucleotidyltransferases of calf thymus nuclei. J. Biol. Chem. 241 (1966) 4339-4352; and Krakow, J. S., Coutsogeorgopoulos, C. and Canellakis, E. S. Studies on the incorporation of deoxyribonucleic acid. Biochim. Biophys. Acta 55 (1962) 639-650, among others.

Additional polymerases with the ability to extend single stranded nucleic acids in the absence of template that can be used include, but are not limited to, Polymerase Theta (Kent et al., eLife 5 (2016): e13740.), polymerase mu (Juarez et al., Nucleic acids research 34.16 (2006): 4572-4582.; or McElhinny et all., Molecular cell 19.3 (2005): 357-366.) or polymerases where template independent activity is induced, e.g. by the insertion of elements of a template independent polymerase (Juarez et al., Nucleic acids research 34.16 (2006): 4572-4582).

In other DNA synthesis applications, the polymerase can be a template-dependent polymerase i.e., a DNA-directed DNA polymerase (e.g., an enzyme having activity 2.7.7.7 using the IUBMB nomenclature) or an RNA-directed DNA polymerase. A description of such enzymes can be found in Richardson, A. Enzymatic synthesis of deoxyribonucleic acid. XIV. Further purification and properties of deoxyribonucleic acid polymerase of Escherichia coli. J. Biol. Chem. 239 (1964) 222-232; Schachman, A. Enzymatic synthesis of deoxyribonucleic acid. VIL Synthesis of a polymer of deoxyadenylate and deoxythymidylate. J. Biol. Chem. 235 (1960) 3242-3249; and Zimmerman, B. K. Purification and properties of deoxyribonucleic acid polymerase from Micrococcus lysodeikticus. J. Biol. Chem. 241 (1966) 2035-2041.

In some embodiments, the polymerase comprises an RNA polymerase. In these embodiments, a RNA specific nucleotidyl transferase, such as E. coli Poly(A) Polymerase (IUBMB EC 2.7.7.19) or Poly(U) Polymerase, among others, may be employed. The RNA nucleotidyl transferases can contain modifications, e.g., single point mutations, that influence the substrate specificity towards a specific rNTP (Lunde et al., Nucleic acids research 40.19 (2012): 9815-9824.). In some embodiments, a very short tether between an RNA nucleotidyl transferase and a ribonucleotide may be used to induce a high effective concentration of the nucleotide, thereby forcing incorporation of an rNTP that might not be the natural substrate of the nucleotidyl transferase.

### Nucleotides

The term “nucleotides” and related terms refers to a molecule comprising an aromatic base, a five carbon sugar (e.g., ribose or deoxyribose), and at least one phosphate group. Canonical or non-canonical nucleotides are consistent with use of the term. The phosphate in some embodiments comprises a monophosphate, diphosphate, or triphosphate, or corresponding phosphate analog.

Nucleotides (and nucleosides) typically comprise a hetero cyclic base including substituted or unsubstituted nitrogen-containing parent heteroaromatic ring which are commonly found in nucleic acids, including naturally-occurring, substituted, modified, or engineered variants, or analogs of the same. Exemplary bases include, but are not limited to, purines and pyrimidines such as: 2-aminopurine, 2,6-diaminopurine, adenine (A), ethenoadenine, N6-Δ2-isopentenyladenine (6iA), N6-Δ2-isopentenyl-2-methylthioadenine (2ms6iA), N6-methyladenine, guanine (G), isoguanine, N2-dimethylguanine (dmG), 7-methylguanine (7mG), 2-thiopyrimidine, 6-thioguanine (6sG), hypoxanthine and O6-methylguanine; 7-deaza-purines such as 7-deazaadenine (7-deaza-A) and 7-deazaguanine (7-deaza-G); pyrimidines such as cytosine (C), 5-propynylcytosine, isocytosine, thymine (T), 4-thiothymine (4sT), 5,6-dihydrothymine, O4-methylthymine, uracil (U), 4-thiouracil (4sU) and 5,6-dihydrouracil (dihydrouracil; D); indoles such as nitroindole and 4-methylindole; pyrroles such as nitropyrrole; nebularine; inosines; hydroxymethylcytosines; 5-methycytosines; base (Y); as well as methylated, glycosylated, and acylated base moieties; and the like. Additional exemplary bases can be found in Fasman, 1989, in “Practical Handbook of Biochemistry and Molecular Biology”, pp. 385-394, CRC Press, Boca Raton, Fla.

Nucleotides (and nucleosides) typically comprise a sugar moiety, such as carbocyclic moiety (Ferraro and Gotor 2000 Chem. Rev. 100: 4319-48), acyclic moieties (Martinez, et al., 1999 Nucleic Acids Research 27: 1271-1274; Martinez, et al., 1997 Bioorganic & Medicinal Chemistry Letters vol. 7: 3013-3016), and other sugar moieties (Joeng, et al., 1993 J. Med. Chem. 36: 2627-2638; Kim, et al., 1993 J. Med. Chem. 36: 30-7; Eschenmosser 1999 Science 284:2118-2124; and U.S. Pat. No. 5,558,991). The sugar moiety comprises: ribosyl; 2′-deoxyribosyl; 3′-deoxyribosyl; 2′,3′-dideoxyribosyl; 2′,3′-didehydrodideoxyribosyl; 2′-alkoxyribosyl; 2′-azidoribosyl; 2′-aminoribosyl; 2′-fluororibosyl; 2′-mercaptoriboxyl; 2′-alkylthioribosyl; 3′-alkoxyribosyl; 3′-azidoribosyl; 3′-aminoribosyl; 3′-fluororibosyl; 3′-mercaptoriboxyl; 3′-alkylthioribosyl carbocyclic; acyclic or other modified sugars.

In some embodiments, nucleotides comprise a chain of one, two or three phosphorus atoms where the chain is typically attached to the 5′ carbon of the sugar moiety via an ester or phosphoramide linkage. In some embodiments, the nucleotide is an analog having a phosphorus chain in which the phosphorus atoms are linked together with intervening O, S, NH, methylene or ethylene. In some embodiments, the phosphorus atoms in the chain include substituted side groups including O, S or BH3. In some embodiments, the chain includes phosphate groups substituted with analogs including phosphoramidate, phosphorothioate, phosphordithioate, and O-methylphosphoroamidite groups.

In some embodiments, the polymerase of the conjugate may be covalently attached to oligonucleotides or nucleotides via the nucleotide base. For example, the nucleotide or oligonucleotide may have the polymerase attached to the C5 position of a pyrimidine base or the C7 position of a 7-deaza purine base through a linker moiety.

A nucleotide used in the present disclosure can also include native or non-native bases. In this regard a native deoxyribonucleic acid can have one or more bases selected from the group consisting of adenine, thymine, cytosine or guanine and a ribonucleic acid can have one or more bases selected from the group consisting of uracil, adenine, cytosine or guanine. Exemplary non-native bases that can be included in a nucleic acid, whether having a native backbone or analog structure, include, without limitation, inosine, xathanine, hypoxathanine, isocytosine, isoguanine, 5-methylcytosine, 5-hydroxymethyl cytosine, 2-aminoadenine, 6-methyl adenine, 6-methyl guanine, 2-propyl guanine, 2-propyl adenine, 2-thioLiracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like.

In some embodiments, the phosphorylated nucleoside (e.g., nucleotide) to be tethered to the polymerase is a nucleoside comprising at least one phosphate group. In some embodiments, the nucleoside comprises at least 1, 2, 3, 4, 5, 6, 7, 8, 9, or more than 9 phosphate groups. In some embodiments, the nucleoside comprises at least 3 phosphate groups. In some embodiments, the phosphorylated nucleoside is adenosine, cytidine, uridine, or guanosine, each of which comprises at least one phosphate group. In some embodiments, the phosphorylated nucleoside is a deoxynucleoside comprising at least one phosphate group. In some embodiments, the phosphorylated nucleoside is a deoxynucleoside comprising at least 3 phosphate groups. In some embodiments, the deoxynucleoside comprises at least 1, 2, 3, 4, 5, 6, 7, 8, 9, or more than 9 phosphate groups. In some embodiments, the phosphorylated nucleoside is deoxyadenosine, deoxycytidine, deoxythymidine, or deoxyguanosine, each of which comprises at least one phosphate group. In some embodiments, the phosphorylated nucleoside is a nucleoside triphosphate, such as dNTP. In some embodiments, the phosphorylated nucleoside is a nucleoside tetraphosphate, nucleoside pentaphosphate, a nucleoside hexaphosphate, a nucleoside heptaphosphate, nucleoside octaphosphate, or a nucleoside nonaphosphate. In some embodiments, the phosphorylated nucleoside is a nucleoside hexaphosphate. In some embodiments, the phosphorylated nucleoside is a nucleoside triphosphate. In some embodiments, the phosphorylated nucleoside is selected from the group consisting of deoxyadenosine triphosphate (dATP), deoxyguanosine triphosphate (dGTP), deoxycytidine triphosphate (dCTP), deoxythymidine triphosphate (dTTP), deoxyadenosine tetraphosphate, deoxyguanosine tetraphosphate, deoxycytidine tetraphosphate, deoxythymidine tetraphosphate, deoxyadenosine pentaphosphate, deoxyguanosine pentaphosphate, deoxycytidine pentaphosphate, deoxythymidine pentaphosphate, deoxyadenosine hexaphosphate, deoxyguanosine hexaphosphate, deoxycytidine hexaphosphate, deoxythymidine hexaphosphate, and any combination thereof.

In some embodiments, the nucleotides analogs described herein comprise a reversible terminator group, such as such as an O-azidomethyl or O—NH2 group on the 3′ position of the sugar or an (alpha-tertbutyl-2-nitrobenzyl)oxymethl group on the 5 position of pyrimidines or the 7 position of 7-deazapurines (for an overview see, e.g. Chen et al., Genomics, Proteomics & Bioinformatics 2013 11: 34-40). In these embodiments, the nucleotide analog prevents or hinders further elongation once incorporated into a nucleic acid to achieve controlled termination of synthesis. In some embodiments, when used as part of a conjugate, the RTdNTP-polymerase conjugates do not rely on the shielding effect to achieve termination, e.g. when a 3′ modified RTdNTP is tethered to the polymerase, the linker used may exceed 100 Å or 200 Å in length.

### Methods

The above described conjugates can be used in a method of nucleic acid synthesis. Nucleic acid synthesis can refer to synthesis, or generation of a product that is a nucleic acid molecule (e.g. a polynucleotide). Methods of nucleic acid synthesis can comprise stepwise synthesis, wherein nucleotides are inserted stepwise into a nucleic acid polymer or polynucleotide. By way of non-limiting example, one typical process for stepwise synthesis of a polynucleotide comprises adding nucleotides stepwise to a starter molecule (e.g., an initial oligonucleotide) via the cycled steps of: addition of a polymerase-nucleotide conjugate to an oligonucleotide under conditions suitable for covalently binding the nucleotide to the end of the oligonucleotide catalyzed by the polymerase. Successful incorporation of a nucleotide of a conjugate into an oligonucleotide can be referred to as an “extension” or “extension reaction,” which generates an “extension product.”

In some embodiments, this method comprises: incubating a nucleic acid with a first conjugate under conditions in which the polymerase catalyzes the covalent addition of the nucleotide of the first conjugate onto the 3′ hydroxyl of the nucleic acid, to make an extension product. This reaction can be performed using a nucleic acid that is attached to a solid support or that is in solution, e.g., not tethered to a solid support. Addition of the conjugate to the nucleic acid results in a nucleic acid with an added 3′ group that is shielded by the linked polymerase, inhibiting subsequent addition of another nucleotide while the polymerase is attached. After elongation of the nucleic acid by the first desired nucleotide, the method comprises a deprotection (de-shielded) step wherein the cleavable linkage of the linker is cleaved, thereby releasing the polymerase from the extension product. Cleavage of the linker removes the polymerase to produce a deprotected extension product. Deprotection enables subsequent extension of the nucleic acid, and thus allows these steps to be repeated cyclically to produce an extension product of defined sequence. Specifically, in some embodiments, the method may further comprise, after deprotection: incubating the deprotected extension product with a second conjugate under conditions in which polymerase catalyzes the covalent addition of the nucleotide of the second conjugate onto the 3′ end of the extension product.

In some embodiments, the method may involve (a) incubating a nucleic acid with a first conjugate under conditions in which the polymerase catalyzes the covalent addition of the nucleotide of the first conjugate (i.e., a single nucleotide) onto the 3′ hydroxyl of the nucleic acid, to make an extension product; (b) cleaving the cleavable linkage of the linker, thereby releasing the polymerase from the extension product and deprotecting the extension product; (c) incubating the deprotected extension product with a second conjugate of claim 1 under conditions in which the polymerase catalyzes the covalent addition of the nucleotide of the second conjugate onto the 3′ end of the extension product, to make a second extension product; (d) repeating steps (b)-(c) on the second extension product multiple times (e.g., 2 to 100 or more times) to produce an extended oligonucleotide of a defined sequence. Steps (b)-(c) may be repeated as many times as necessary until an extension product of a defined sequence and length is synthesized. The end product may be 2-100 bases in length, although, in theory, the method can be used to produce products of any length, including greater than 200 bases or greater than 500 bases.

In some embodiments, methods of nucleic acid synthesis as provided herein are carried out in a reaction buffer composition. In some embodiments, the reaction buffer composition is an aqueous solution. In some embodiments, the reaction buffer composition comprises a set of components suitable for the stability of the polymerase, nucleotide, polymerase-nucleotide conjugates, starter molecule, nucleic acid molecule products, and any surface or matrix on which the methods disclosed herein are carried out. In some such embodiments, the reaction buffer composition comprises a set of components suitable for carrying out catalytic steps (e.g. polynucleotide polymerization performed by a polymerase) described in methods of nucleic acid synthesis in accordance with the present disclosure.

The conditions under which nucleic acid synthesis is carried out can be varied. For example, the amounts of times for carrying out each step in a stepwise nucleotide addition cycle can be varied to improve the purity of a plurality of products generated by the methods of nucleic acid synthesis described herein.

In some embodiments, methods of nucleic acid synthesis in accordance with the present disclosure generate a nucleic acid molecule product (i.e. a polynucleotide product). In some embodiments, the nucleic molecule product (i.e. polynucleotide product) has a target (i.e. pre-determined) sequence. A “target” or “pre-determined” sequence refers to a desired polynucleotide sequence that is intentionally produced by the method of nucleic acid synthesis. The pre-determined sequence can include any number of nucleotides comprising a nucleobase (e.g. adenine, thymine, guanine, cytosine, and/or uracil). In some embodiments, the nucleotide is a modified nucleotide (i.e. a nucleotide analog). In some embodiments, the nucleobase is a modified nucleobase. In some embodiments, the pre-determined sequence contains one or more designated positions which may be a random nucleobase. Inclusion of a position with a random nucleobase can be useful, for example, in introducing randomized mutation into a polynucleotide product.

In some embodiments, the present disclosure includes a method of synthesizing a polynucleotide comprising contacting a precursor polynucleotide with a conjugate comprising a nucleotide covalently linked to a polymerase via a cleavable linker, wherein said nucleotide comprises said protected nucleobase. In some embodiments, the method of synthesizing a polynucleotide comprises cleaving a cleavable linker after addition of a nucleotide to a precursor polynucleotide. In some embodiments, the method of synthesizing a polynucleotide comprises repeating contacting, adding, and optionally cleaving steps described herein one or more times. In some embodiments, removal of one or more protecting groups described herein comprises contacting said polynucleotide with an enzyme capable of removing said one or more protecting groups from said protected nucleobases. In some embodiments, a method of synthesizing a polypeptide comprising contacting said polynucleotide with two or more enzymes capable of removing said one or more protecting groups from said protected nucleobases.

In some embodiments, synthesis of a polynucleotide comprises adding nucleotides stepwise to a starter molecule (e.g., an initial oligonucleotide) via the cycled steps of: addition of polymerase-nucleotide conjugate to an oligonucleotide, binding of the nucleotide to the 3′ end of the oligonucleotide catalyzed by the polymerase, and cleavage of the polymerase from the added nucleotide. These steps can be repeated until a desired polynucleotide is synthesized. As described herein, the use of nucleotides comprising protected nucleobases during polynucleotide synthesis helps to improve the efficiency and accuracy of synthesis by inhibiting secondary structure formation which can interfere with the addition of the incoming nucleotide by the polymerase during synthesis.

Although synthesis can be completed entirely with protected nucleotides, synthesis with a combination of unmodified and protected nucleotides can also be used effectively to improve polynucleotide synthesis. In some embodiments, only one of the four nucleotides added (e.g., from G or T) is protected during synthesis. In some embodiments, protected nucleotides are only added at targeted positions where secondary structure or ternary structure is predicted, which could interfere with synthesis. Such structures can be predicted based on the presence of complementary DNA regions in various ways and respective tools exist, such as the NUPACK algorithms (http://www.nupack.org/home/model). Thus, in some embodiments, synthesis of a completed polynucleotide where synthesis is improved can include the use of only 1 or 2 protected nucleotides. In some embodiments, about 5%, about 10%, about 20%, about 30%, about 50%, substantially all, or 100% of a specific nucleotide is incorporated into the polynucleotide in their protected version. In some embodiments, less than 5%, less than 10%, less than 20%, less than 30%, or less than 50% of a specific nucleotide is incorporated into the polynucleotide in its protected version. In some embodiments, more than 5%, more than 10%, more than 20%, more than 30%, or more than 50% of a specific nucleotide is incorporated into the polynucleotide in its protected version. In some embodiments, only protected guanine nucleotides are used in the nucleotide synthesis reaction. The removal of protecting groups in the terminal positions of a nucleic acid may be more challenging than the removal from internal DNA positions. Therefore, in some embodiments, nucleotide synthesis is performed such that the last and first 1, 2, or 3 positions of the synthesized nucleic acid does not comprise protected nucleotides.

A nucleic acid molecule product or polynucleotide product generated by the methods described herein can contain a plurality of products. In some embodiments, the plurality of products comprises a nucleic acid molecule comprising the target (i.e. pre-determined) sequence. In some embodiments, the plurality of products comprises a nucleic acid molecule comprising a sequence that is not the target sequence. In some embodiments, the plurality of products comprises a nucleic acid molecule product comprising the target sequence and a nucleic acid molecule product that is not the target sequence. The “purity” of the plurality of products can refer to the ratio of the abundance of nucleic acid molecule products with the target sequence to the abundance of nucleic acid molecule products that do not have the target sequence. The purity of a product can be assessed by any number of methods known in the art for determining the sequence of a nucleic acid. Any suitable nucleic acid sequencing method can be used. For example, the product can be assessed, without limitation, by Sanger sequencing, next generation sequencing (e.g. Illumina sequencing), or long-read sequencing (e.g. small molecule, real-time sequencing (SMRT) and nanopore sequencing).

In some embodiments, a method of nucleic acid synthesis in accordance with the present disclosure produces a product having a purity between about 10% and about 99.99%. In some embodiments, the method of nucleic acid synthesis produces a product having a purity of at least 10%. In some embodiments, the method of nucleic acid synthesis produces a product having a purity of at least 10%. In some embodiments, the method of nucleic acid synthesis produces a product having a purity of at least 20%. In some embodiments, the method of nucleic acid synthesis produces a product having a purity of at least 30%. In some embodiments, the method of nucleic acid synthesis produces a product having a purity of at least 40%. In some embodiments, the method of nucleic acid synthesis produces a product having a purity of at least 50%. In some embodiments, the method of nucleic acid synthesis produces a product having a purity of at least 60%. In some embodiments, the method of nucleic acid synthesis produces a product having a purity of at least 70%. In some embodiments, the method of nucleic acid synthesis produces a product having a purity of at least 80%. In some embodiments, the method of nucleic acid synthesis produces a product having a purity of at least 90%. In some embodiments, the method of nucleic acid synthesis produces a product having a purity of at least 95%. In some embodiments, the method of nucleic acid synthesis produces a product having a purity of at least 99%.

In any of the above-summarized embodiments, the nucleoside triphosphate may be a deoxyribonucleoside triphosphate or a ribonucleoside triphosphate. In some embodiments, a conjugate may comprise an RNA polymerase linked to a ribonucleoside triphosphate. In these embodiments, the nucleotide added to the nucleic acid may be a ribonucleotide. In other embodiments, a conjugate comprises an DNA polymerase linked to a deoxyribonucleoside triphosphate. In these embodiments, the nucleotide added to the nucleic acid may be a deoxyribonucleotide.

In some embodiments, the nucleotide is a nucleotide analog. In some embodiments, the nucleotide analog is a reversible terminator. Reversible terminators are known in the art for use in nucleic acid synthesis. Uses of reversible terminators in nucleic acid synthesis have been described previously; see, for example, WO 2021/122539 A1, WO 2018/215803 A1, WO 2021/094251 A1, and WO 2020/081985 A1.

In some embodiments, the nucleotide may be comprise a reversible terminator (“RTdNTP”) and the deprotection step of the method further comprises removing the blocking group (e.g., removing the terminator group) from the added nucleotide to produce the deprotected extension product. Deprotection enables subsequent extension of the nucleic acid, and thus allows these steps to be repeated cyclically to produce an extension product of defined sequence.

A method of sequencing is also provided. These methods may comprise incubating a duplex comprising a primer and a template with a composition comprising a set of conjugates, wherein the conjugates correspond to G, A, T and C and are distinguishably labeled, e.g., fluorescently labeled; detecting which nucleotide has been added to the primer by detecting a label that is tethered to the polymerase that has added the nucleotide to the primer; deprotecting the extension product by cleaving the linker; and repeating the incubation, detection and deprotection steps to obtain the sequence of at least part of the template.

### Conjugate Synthesis

In some examples, a polymerase-nucleotide conjugate is prepared by first synthesizing an intermediate compound comprising a linker and a nucleotide (referred to herein as a “linker-nucleotide”), and then this intermediate compound is attached to the polymerase.

A person of ordinary skill in the art will understand the conjugates and nucleotides disclosed herein can be prepared in a manner similar to the reaction schemes shown below. The synthetic approaches outlined in these reaction schemes may be illustrated for specific nucleotides. Similar synthetic approaches can be applied to related nucleotide analogs.

There are several known reactions and functional groups suitable for generating nucleotide polymerase conjugates having cleavable linkers conforming to those describe herein. For example connection of the conjugate components can be achieved by the formation of a disulfide (forming a readily cleavable connection), formation of an amide, formation of an ester, protein-ligand linkage (e.g., biotin-streptavidin linkage), by alkylation (e.g., using a substituted iodoacetamide reagent) or forming adducts using aldehydes and amines or hydrazines.

In some embodiments, the separate components of the conjugate comprise a site suitable for conjugation to facilitate conjugate synthesis (i.e., a conjugate group). Examples of such conjugate groups include but are limited to hydroxyl, ester, amine, carbonate, acetal, aldehyde, aldehyde hydrate, alkenyl, acrylate, methacrylate, acrylamide, active sulfone, hydrazide, thiol, alkanoic acid, acid halide, isocyanate, isothiocyanate, maleimide, vinylsulfone, dithiopyridine, vinylpyridine, iodoacetamide, epoxide, glyoxal, dione, mesylate, tosylate, and tresylate.

Further examples of conjugate groups include —NH₂, —COOH, —COOCH₃, —N-hydroxysuccinimide, and -maleimide. In some embodiments, the bioconjugate reactive group may be protected (e.g., with a protecting group). Additional examples of bioconjugate reactive groups and the resulting bioconjugate reactive linkers can be found, e.g., in PCT Publication WO2021/226327, incorporated by reference in its entirety.

### Linker-Nucleotide Synthesis

An exemplary reaction scheme for preparing linker nucleotide conjugates with a linker comprising an amino acid ester, including linkers described herein and variants thereof, is described here. In some embodiments, a desired nucleotide can be commercially obtained, and its hydroxyl groups protected by TBS before conjugation of the L1-OH group to an exocyclic oxygen or amine on the nucleobase. Alternatively, an already modified nucleotide comprising the L1-OH group bound to the nucleotide can be obtained, such as an L1-OH group bound to the C5 of a pyrimidine or an L1-OH group bound to C7 of a 7 deazapurine. An Fmoc-protected amino acid ester is then coupled to the hydroxyl group of L1-OH, followed by hydroxyl group deprotection and triphosphorylation of the nucleoside.

To complete the linker, Fmoc is removed from the amino acid ester amine group and it is coupled to the rest of the linker including L3, which is capable of binding to a polymerase.

### Nucleotide Attachment

In some embodiments, the linker is bound to a portion of the nucleotide at an atom that is not involved in base pairing. In other embodiments, the linker is bound to the nucleobase of the nucleotide at an atom that is involved in base pairing. In some embodiments, the linker is considered to be at least the atoms that connect the polymerase to any atom in the monocyclic or polycyclic ring system bonded to the Γ position of the sugar (e.g. pyrimidine or purine or 7-deazapurine or 8-aza-7-deazapurine).

Certain polymerases have a high tolerance for modification of certain parts of a nucleotide, e.g. modifications of the 5 position of pyrimidines and the 7 position of purines are well-tolerated by some polymerases (He and Seela., Nucleic Acids Research 30.24 (2002): 5485-5496.; or Hottin et al., Chemistry. 2017 Feb. 10; 23(9):2109-2118). In some embodiments, the linker is attached the 5 position of pyrimidines or the 7 position of 7-deazapurines. In other embodiments, the linker may be attached to an exocyclic amine of a nucleobase, e.g. by N-alkylating the exocyclic amine of cytosine with a nitrobenzyl moiety as discussed below.

In other embodiments, the linker is joined to the sugar or to the α-phosphate of the nucleotide. In some embodiments, the linker is jointed to the terminal phosphate of the nucleotide. In all embodiments, the linker used should be sufficiently long to allow the nucleotide to access the active site of the polymerase to which it is tethered. As will be described in greater detail below, the polymerase of a conjugate is capable of catalyzing the addition of the nucleotide to which it is linked onto the 3′ end of a nucleic acid.

Conjugation of nucleotides or other base-pairing moieties to linkers may be achieved by any means known in the art of chemical conjugation methods. Nucleotide bases can be obtained or modified to include an L1 portion of the linker. The rest of the linker can be attached to L1 using methods exemplified herein. Those skilled in the art will know or be able to determine appropriate methods for attaching linkers based on the reactivities of these bases.

In some embodiments, nucleotides containing base modifications that add a free amine group are contemplated for use in conjugation to linkers as described herein. Primary amines, for example, may be linked to the base in such a manner that they can be reacted with heterobifunctional polyethylene glycol (PEG) linkers to create a nucleotide containing a variable length PEG linker. Examples of such amine-containing nucleotides include 5-propargylamino-dNTPs, 5-propargylamino-NTPs, amino allyl-dNTPs, and amino allyl-NTPs.

### Polymerase Attachment

In some embodiments, the tethered nucleotide may be specifically attached to a cysteine residue of the polymerase using a sulfhydryl-specific attachment chemistry. Possible sulfhydryl specific attachment chemistries include, but are not limited to ortho-pyridyl disulfide (OPSS), maleimide functionalities, 3-arylpropiolonitrile functionalities, allenamide functionalities, haloacetyl functionalities such as iodoacetyl or bromoacetyl, alkyl halides or perfluroaryl groups that can favorably react with sulfhydryls surrounded by a specific amino acid sequence (Zhang, Chi, et al. Nature chemistry 8, (2015) 120-128.). Other attachment chemistries for specific labeling of cysteine residues will be apparent to those skilled in the art or are described in the pertinent literature and texts (e.g., Kim, Younggyu, et al, Bioconjugate chemistry 19.3 (2008): 786-791.).

In other embodiments, the linker could be attached to a lysine residue via an amine-reactive functionality (e.g. NHS esters, Sulfo-NHS esters, tetra- or pentafluorophenyl esters, isothiocyanates, sulfonyl chlorides, etc.). In other embodiments, the linker may be attached to the polymerase via attachment to a genetically inserted unnatural amino acid, e.g. p-propargyloxyphenylalanine or p-azidophenylalanine that could undergo azide-alkyne Huisgen cycloaddition, though many suitable unnatural amino acids suitable for site-specific labeling exist and can be found in the literature (e.g. as described in Lang and Chin., Chemical reviews 114.9 (2014): 4764-4806.).

In other embodiments, the linker may be specifically attached to the polymerase N-terminus. In some embodiments, the polymerase is mutated to have an N-terminal serine or threonine residue, which may be specifically oxidized to generate an N-terminal aldehyde for subsequent coupling to e.g. a hydrazide. In other embodiments, the polymerase is mutated to have an N-terminal cysteine residue that can be specifically labeled with an aldehyde to form a thiazolidine. In other embodiments, an N-terminal cysteine residue can be labeled with a peptide linker via Native Chemical Ligation.

In other embodiments, a peptide tag sequence may be inserted into the polymerase that can be specifically labeled with a synthetic group by an enzyme, e.g. as demonstrated in the literature using biotin ligase, transglutaminase, lipoic acid ligase, bacterial sortase and phosphopantetheinyl transferase (e.g. as described in refs. 74-78 of Stephanopoulos & Francis Nat. Chem. Biol. 7, (2011) 876-884).

In other embodiments, the linker is attached to a labeling domain fused to the polymerase. For example, a linker with a corresponding reactive moiety may be used to covalently label SNAP tags, CLIP tags, HaloTags and acyl carrier protein domains (e.g. as described in refs. 79-82 of Stephanopoulos & Francis Nat. Chem. Biol. 7, (2011) 876-884).

In other embodiments, the linker is attached to an aldehyde specifically generated within the polymerase, as described in Carrico et al. (Nat. Chem. Biol. 3, (2007) 321-322). For example, after insertion of an amino acid sequence that is recognized by the enzyme formylglycine-generating enzyme (FGE) into the polymerase, it may be exposed to FGE, which will specifically convert a cysteine residue in the recognition sequence to formylglycine (i.e. producing an aldehyde). This aldehyde may then be specifically labeled with e.g. a hydrazide or aminooxy moiety of a linker.

In some embodiments, a linker may be attached to the polymerase via non-covalent binding of a moiety of the linker to a moiety fused to the polymerase. Examples of such attachment strategies include fusing a polymerase to streptavidin that can bind a biotin moiety of a linker, or fusing a polymerase to anti-digoxigenin that can bind a digoxigenin moiety of a linker. In some embodiments, site-specific labeling may lead to an attachment of the linker to the polymerase that may readily be reversed (e.g. an ortho-pyridyl disulfide (OPSS) group that forms a disulfide bond with a cysteine that can be cleaved using reducing agents, e.g. using TCEP), other attachment chemistries will produce permanent attachments.

In any embodiment, the polymerase may be mutated to ensure specific attachment of the tethered nucleotide to a particular location of the polymerase, as will be apparent to those skilled in the art. For example, with sulfhydryl-specific attachment chemistries such as maleimides or ortho-pyridyl disulfides, accessible cysteine residues in the wild-type polymerase may be mutated to a non-cysteine residue to prevent labeling at those positions. On this “reactive cysteine-free” background, a cysteine residue may be introduced by mutation at the desired attachment position. These mutations preferentially do not interfere with the activity of the polymerase.

In some embodiments, the linker is specifically attached to an amino acid of the polymerase. In these cases, it is preferable to attach the linker to an amino acid at a position that can be mutated without loss of the polymerase activity, e.g. positions 180, 188, 253 or 302 of murine TdT (numbering as in the crystal structure PDB ID: 4127). It is preferable to not attach the linker to an amino acid involved in the catalytic activity of the polymerase to avoid interfering with catalysis. Residues known to be involved with catalysis and methods for determining if a residue is involved with catalysis (e.g. by site-specific mutagenesis) will be apparent to those skilled in the art and are reviewed in literature (e.g. Joyce et al. (Journal of Bacteriology 177.22 (1995): 6321.) and Jara and Martinez (The Journal of Physical Chemistry B 120.27 (2016): 6504-6514.))

Other strategies for site-specific attachment of synthetic groups to proteins will be apparent to those skilled in the art and are reviewed in literature, (e.g. Stephanopoulos & Francis Nat. Chem. Biol. 7, (2011) 876-884).

### EQUIVALENTS AND SCOPE

Those skilled in the art will recognize, or be able to ascertain using no more than routine experimentation, many equivalents to the specific embodiments in accordance with the present disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the present disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

Below are examples of specific embodiments for carrying out the present disclosure. The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3rd Ed. (Plenum Press) Vols A and B(1992).

### General Synthetic Schemes for Conjugates of the Present Disclosure

In some embodiments, conjugates of the present disclosure can prepared as outlined in Scheme 1

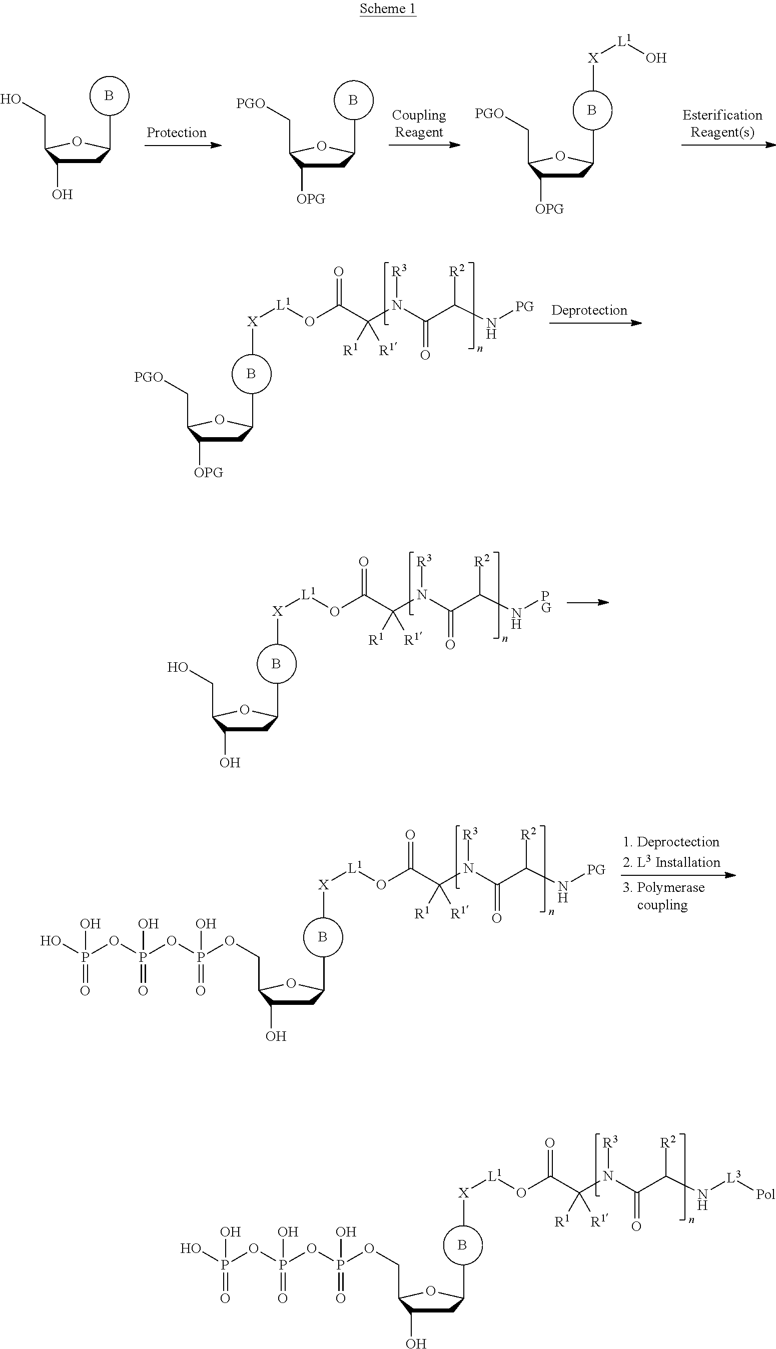

wherein, B is a nucleobase; PG is a protecting group; X is —N— or —O— of the nucleobase; and L¹, L³, R¹, R^{1′}, R², R³, n, and Pol are defined herein.

Nucleotide portion of Polymerase-Nucleotide Conjugates of the present disclosure can be prepared as outlined below:

### Protection of Hydroxyl Groups on Nucleotide

In some embodiments, 3′ hydroxy and 5′ hydroxyl of a nucleotide can be TBS protected. Such a protection reaction can be achieved with TBS-Cl (tert butyldimethylsilyl chloride; TBDMS) in imidazole to form a TBS ester at the 3′ and 5′ locations. For example, protection of the 3′ hydroxy and 5′ hydroxyl of a nucleotide can be achieved as outlined below:

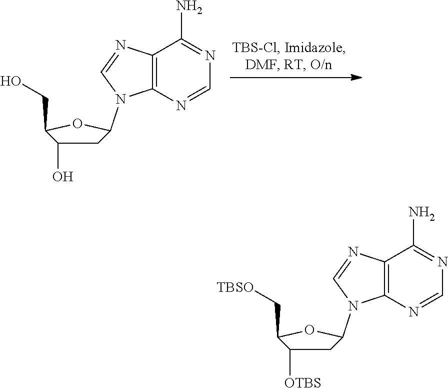 [SMILES: Nc1ncnc2c1ncn2C1CC(O)[C@@H](CO)O1 ; CC(C)(C)[Si](C)(C)OC[C@H]1OC(n2cnc3c(N)ncnc32)CC1O[Si](C)(C)C(C)(C)C]

The protecting group of the protected hydroxy group is not particularly limited and, for example, any protecting group described in GREENE'S PROTECTIVE GROUPS IN ORGANIC SYNTHESIS, 5^{th} ed., JOHN WILLY & SONS (2014), which is incorporated herein by reference in its entirety, and the like can be mentioned. Specifically, methyl group, benzyl group, p-methoxybenzyl group, tert-butyl group, methoxyethyl group, ethoxyethyl group, cyanoethyl group, cyanoethoxymethyl group, phenylcarbamoyl group, 1,1-dioxothiomorpholine-4-thiocarbamoyl group, acetyl group, pivaloyl group, benzoyl group, triethylsilyl group, triisopropylsilyl group, tert-butyldimethylsilyl group, [(triisopropylsilyl)oxy]methyl (Tom) group, 1-(4-chlorophenyl)-4-ethoxypiperidin-4-yl (Cpep) group and the like can be mentioned. The hydroxy-protecting group is preferably a triethylsilyl group, triisopropylsilyl group or tert-butyldimethylsilyl group, more preferably a tert-butyldimethylsilyl group from the aspects of economic efficiency and easy availability. Protection and deprotection of the hydroxy group are well known and can be performed by, for example, the method described in the aforementioned GREENE'S PROTECTIVE GROUPS IN ORGANIC SYNTHESIS.

### Addition of L¹-OH (“Scar”) to the Nucleotide

An exemplary reaction scheme where L¹ is identified as “scar” is shown below:

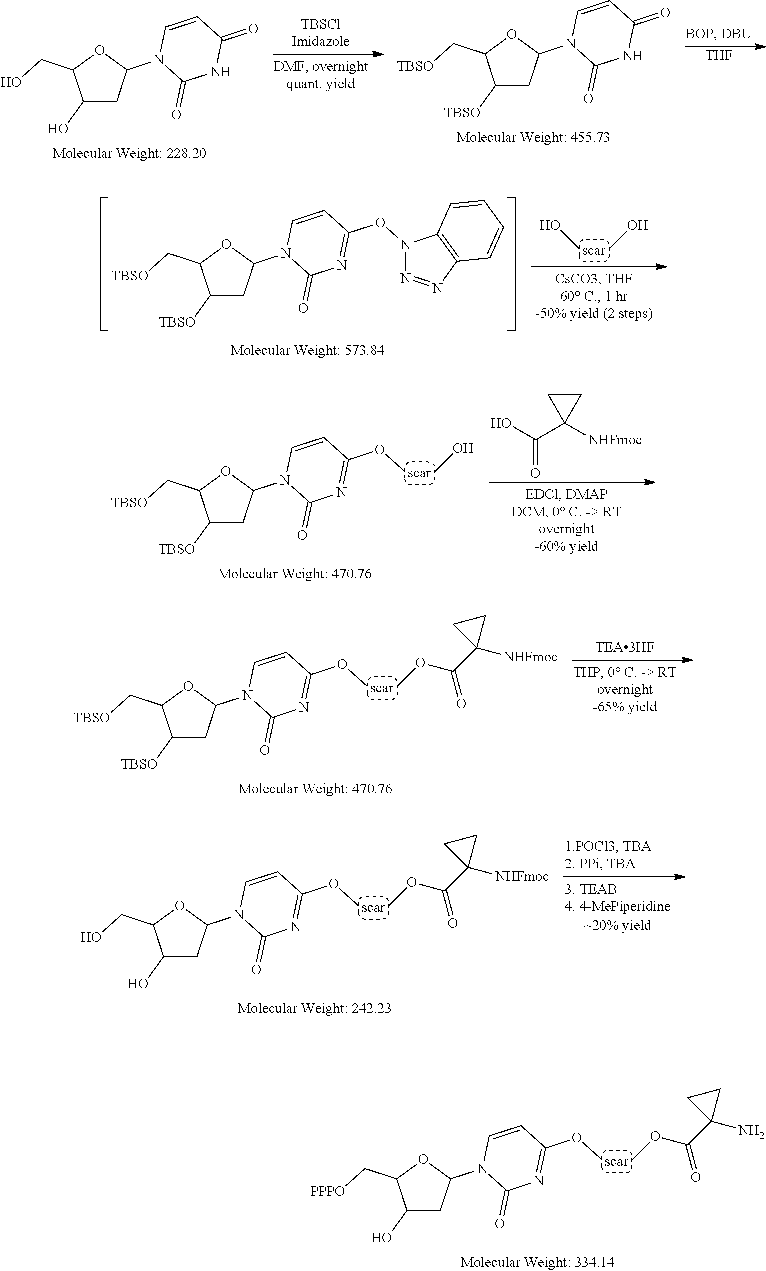

### Guanine

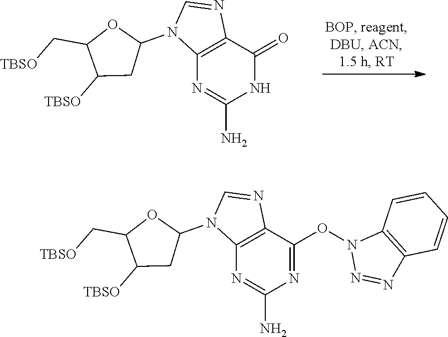 [SMILES: CC(C)(C)[Si](C)(C)OCC1OC(n2cnc3c(=O)[nH]c(N)nc32)CC1O[Si](C)(C)C(C)(C)C ; CC(C)(C)[Si](C)(C)OCC1OC(n2cnc3c(On4nnc5ccccc54)nc(N)nc32)CC1O[Si](C)(C)C(C)(C)C]

To a solution of the TBS-protected nucleoside derivative in acetonitrile, BOP and DBU are added, and the mixture stirred at room temperature. The mixture is then diluted with EtOAc and washed with deionized water followed by brine. The organic layer is dried over anhydrous Na₂SO₄, filtered, and evaporated under reduced pressure. The obtained residue is dissolved in DCM and then added to Hexane. An oily viscous layer on bottom and a turbid hexane layer on top are formed. The hexane layer is decanted over sodium sulphate. The same hexane wash procedure is repeated two more times with the oily layer. The hexane layers are collected and evaporated under reduced pressure to obtain the intermediate as a white foam. This product used as such for the next reactions.

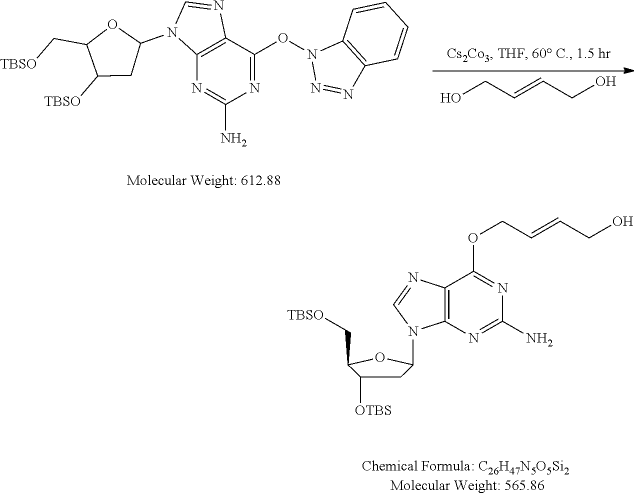 [SMILES: CC(C)(C)[Si](C)(C)OCC1OC(n2cnc3c(On4nnc5ccccc54)nc(N)nc32)CC1O[Si](C)(C)C(C)(C)C ; OC/C=C/CO ; CC(C)(C)[Si](C)(C)OC[C@H]1OC(n2cnc3c(OC/C=C/CO)nc(N)nc32)CC1O[Si](C)(C)C(C)(C)C]

The intermediate is dissolved in dry THF Cs₂CO₃ and E-Butene-1,4-diol are added, and the mixture stirred at 60° C. The reaction mixture is diluted with EtOAc and washed with deionized water followed by brine. The organic layer is dried over anhydrous Na₂SO₄, filtered, and evaporated under reduced pressure. The crude material is purified by chromatography on a silica gel column using a 20-50% EtOAC/hexane gradient to give the TBS-protected, modified nucleoside comprising the scar as a white foam.

The above reaction is specific for addition of E-Butene-1,4-diol, however, many other L1-OH groups can be added by one of ordinary skill in the art using guidelines from the above reaction.

### Thymine

A reaction scheme for adding L1-OH to the O4 of thymine and aligning with the O6 conjugation for guanine is provided below. An example of an alternative L1-OH group is also shown.

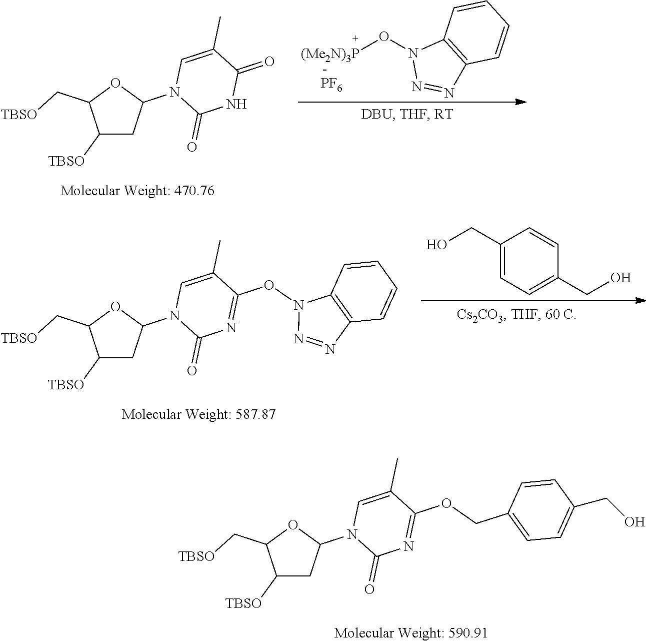

### Uracil

Similar to thymine, the reaction scheme for adding L1-OH to the O4 of uracil can be performed as follows:

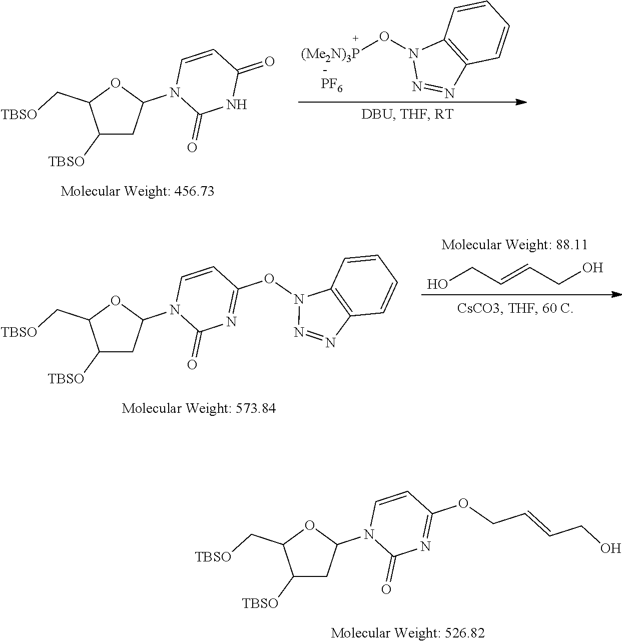

### Adenine

A reaction scheme for adding an L1-OH group to the N6 group of adenine is provided below. Note this scheme also includes the amino acid ester already bound to the L1 group.

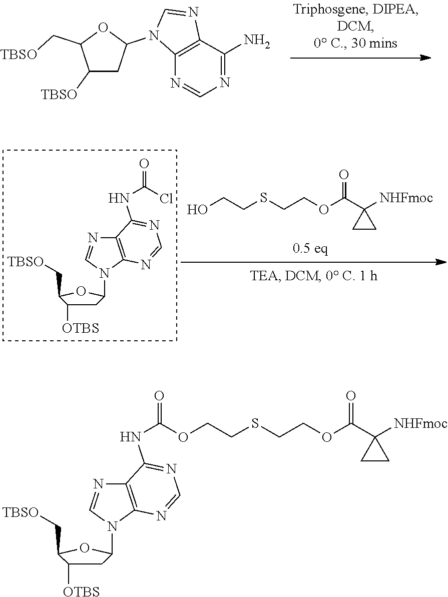

### Cytosine

A reaction scheme for adding an L¹-OH group to the N4 group of cytosine is provided below. Note this scheme also includes the amino acid ester already bound to the L1 group.

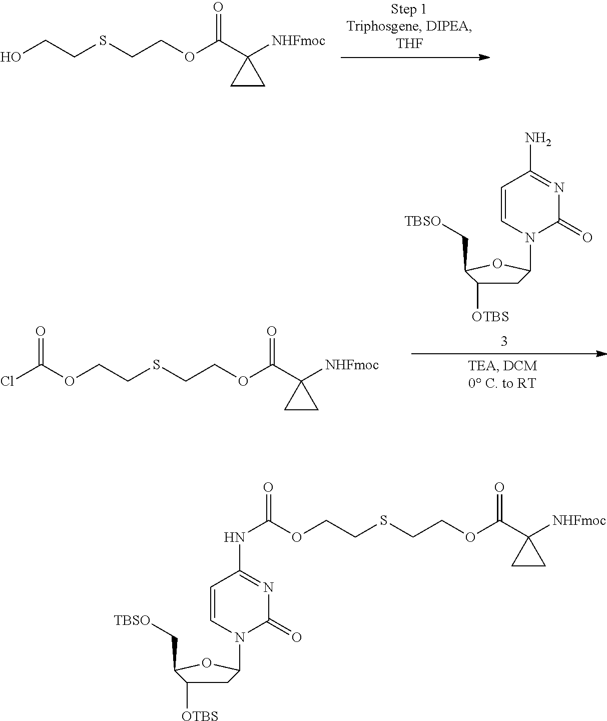

These nucleotides are also commercially available as deoxyribonucleoside triphosphates.

### Coupling Amino Acid Ester

Shown and described below is an exemplary reaction scheme for coupling an amino acid ester to the hydroxyl of the L1-OH group bound to the nucleotide. Although an amino acid ester with a cyclopropyl R group at the alpha carbon is shown, any natural or synthetic amino acid ester with different R groups (e.g., as shown in Example 6) may be used. Similarly, this reaction can be used to guide addition of the amino acid ester to the L1-OH group attached to any nucleotide.

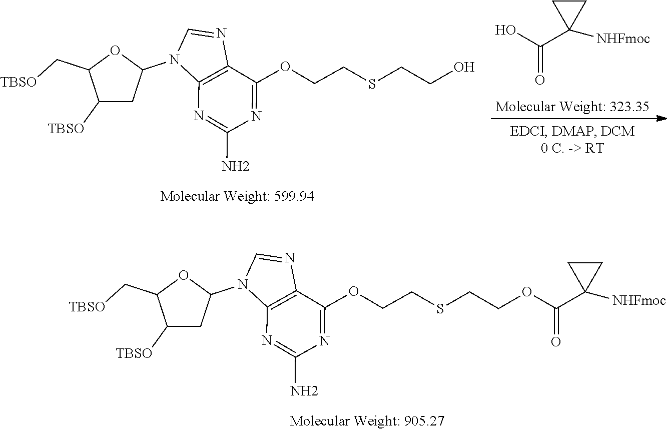

To a solution of the nucleotide L1-OH compound in dry DCM (0.2 M) shown above was added DMAP (0.6 eq) and the Fmoc amino acid (1.2 eq) under inert atmosphere at ambient temperature. The reaction mixture was cooled to 0° and then EDC·HCl was added slowly. The reaction mixture was allowed to stir for 16 hr at ambient temperature. The reaction solution was then diluted with additional DCM and extracted with water. The aqueous layer was then washed with more DCM (2×). The combined organic layer was then washed with sat. ammonium bicarb, dried over Na₂SO₄, and concentrated. The crude material was purified by chromatography on a silica gel column using a 10-35% EtOAC/hexane gradient.

### OH Deprotection

In some embodiments, TBS protected hydroxyl groups can be deprotected using the following scheme.

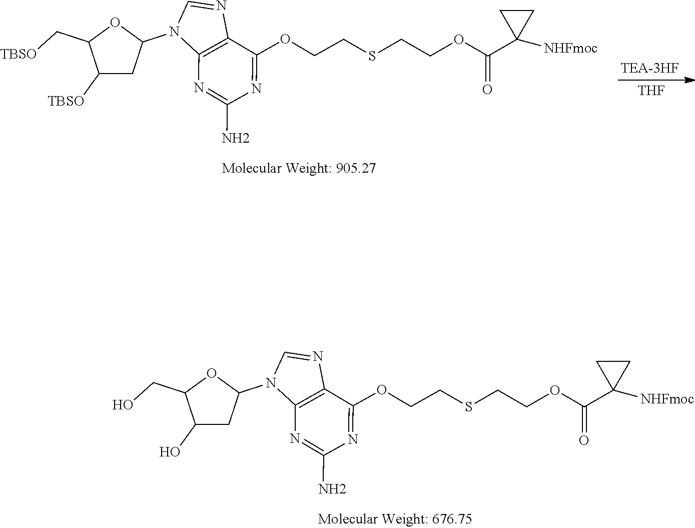

To a 0.1 M solution of protected nucleosides in dry THF at 0° C., was added 3HF-TEA (10 equiv) dropwise. The mixture was stirred for 16 hr while warming to ambient temperature. The reaction was quenched with the addition of a few drops of MeOH, and the solvents were removed under reduced pressure. The residue was diluted with DCM and washed with water. The aqueous layer was again extracted with DCM (1×). The combined organic layer was dried over anhydrous Na₂SO₄, filtered, and concentrated. The compound was purified by chromatography on a silica gel column using a 1-7% MeOH/DCM gradient. All the compounds were obtained as white foamy solids.

### Triphosphorylation and Fmoc Deprotection

A reaction scheme for triphosphorylation of the nucleotide analogue and deprotection of the amino acid ester is shown below:

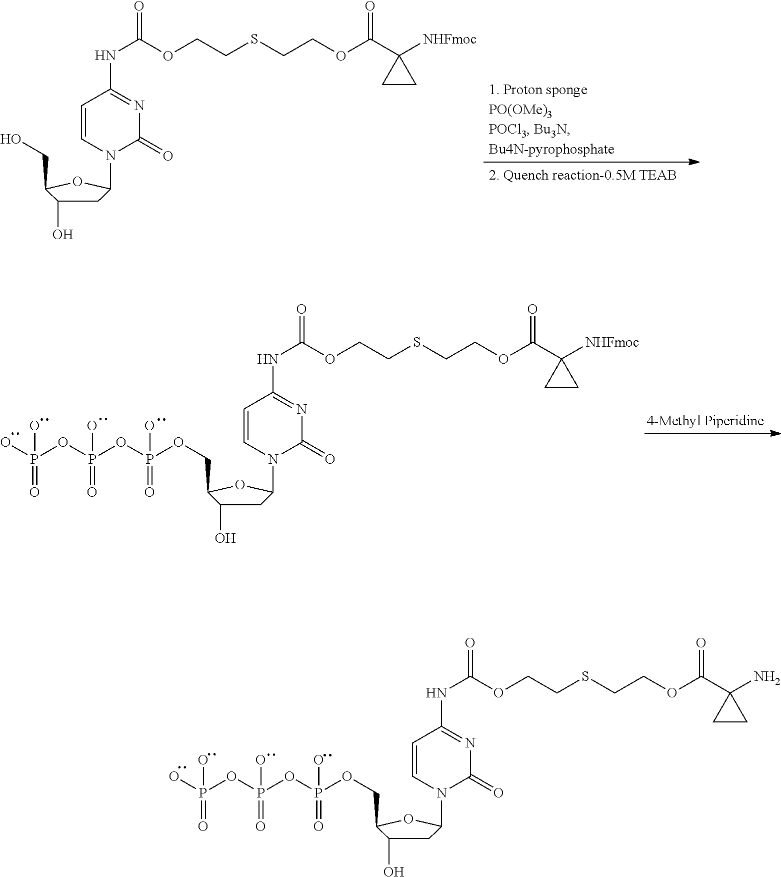

For the triphosphrolyation reaction, nucleoside analogue was placed into a 10 mL round bottom flask equipped with a stir bar and tetrabutylammonium pyrophosphate was placed in a separate 5 mL conical tube. The two flasks were placed in a vacuum desiccator with P₂O₅ and allowed to dry under vacuum for at least 16 hr. Additionally, molecular sieves and three small round bottom flasks were placed in a drying oven for at least 16 hr. Two small flasks from the oven were charged with molecular sieves and flame-activated under vacuum. While these were cooling, the other small flask was attached to a Hickman distillation apparatus and flame dried. Upon cooling, the first two flasks were backfilled with nitrogen. Trimethyl phosphate and tributyl amine were then placed over the molecular sieves in the initial two flasks for drying. The Hickman distillation apparatus was then used to freshly distill POCl₃. The vacuum desiccator was purged with N₂ gas, and the flasks inside were then transferred to nitrogen balloons or the Schlenk line. Trimethyl phosphate (40 eq) was added to the nucleoside and the mixture was cooled at −5° C. To this nucleoside mixture was added dry tributyl amine (3 eq) followed by POCl₃ (2.1, 1.3, 1.5, 1.8 eq respectively) slowly via micro syringe. The combined mixture was stirred at −5° C. for 45 mins. After 45 min, the reaction mixture was treated with a mixture of tributylamine pyrophosphate (5 eq, 0.5 M in dry acetonitrile) and tributyl amine (6 eq). After 1 hour, the mixture was treated with triethylammonium bicarbonate (0.5 M, 1:2 of the total reaction volume) and allowed to stir at ambient temperature for 1 hour. Then, this reaction mixture was further treated with N-Methyl piperidine (⅕^{th} of total reaction volume) and stirred for 90 mins at ambient temperature followed by extraction with dichloromethane (2×). The aqueous layer was then purified by reverse phase HPLC (0.1 M triethylammonium acetate buffer/Acetonitrile, 4-47%, 0-15 min, flow 5 ml min⁻¹). Product containing fractions were pooled and lyophilized to provide desired product as a triethylammonium salt. The resulting solid was reconstituted in RNase Free DI water for further experiments.

### Addition Amino Acids/L3 to Amino Acid Ester

The L3 portion of the linker that binds to the polymerase, along with any amino acids adjacent to the amino acid ester and part of the L2 portion of the linker, can be added using the exemplary reaction scheme below:

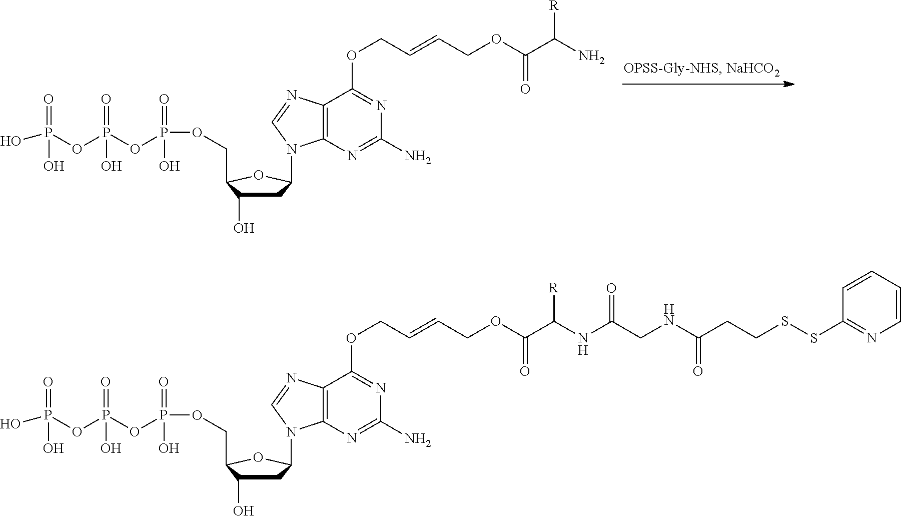

OPSS-Gly-NHS can be synthesized according to the following reaction scheme

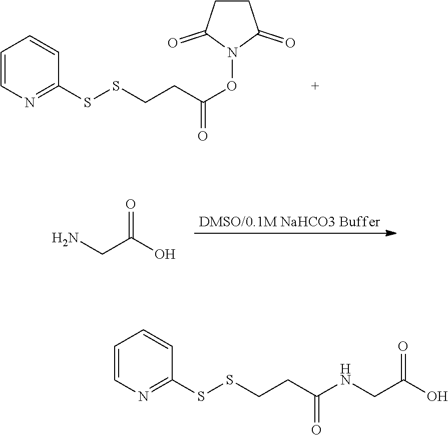 [SMILES: O=C(CCSSc1ccccn1)ON1C(=O)CCC1=O ; NCC(=O)O ; O=C(O)CNC(=O)CCSSc1ccccn1]

This reaction scheme can also be used for addition of more amino acids, or for including alternative amino acids in L2, such as alanine:

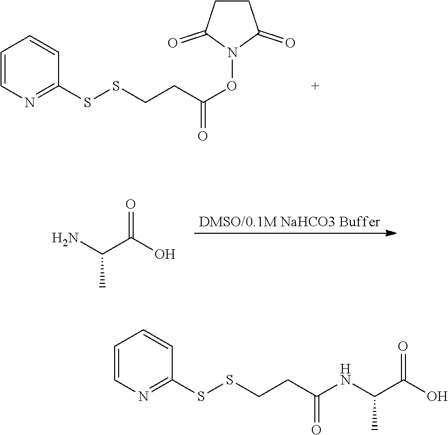

For addition of one or more amino acids to the linker, peptide synthesis can also be carried out using standard solid phase or solution phase chemistry, as desired. Methods for peptide synthesis are well known to those skilled in the art (Fodor et. al., Science 251:767 (1991); Gallop et al., J. Med. Chem. 37:1233-1251 (1994); Gordon et al., J. Med. Chem. 37:1385-1401 (1994)). It is understood that a peptide linker can be synthesized and then added to the NTP as a peptide or can be synthesized by sequentially adding amino acids

### Scheme 2: Synthesis of OPSS-Gly-ACC-EtS-dGTP

A complete exemplary reaction scheme for synthesizing a nucleotide bound to a linker (L1-L2-L3) comprising an amino acid ester as part of the L2 portion is shown below:

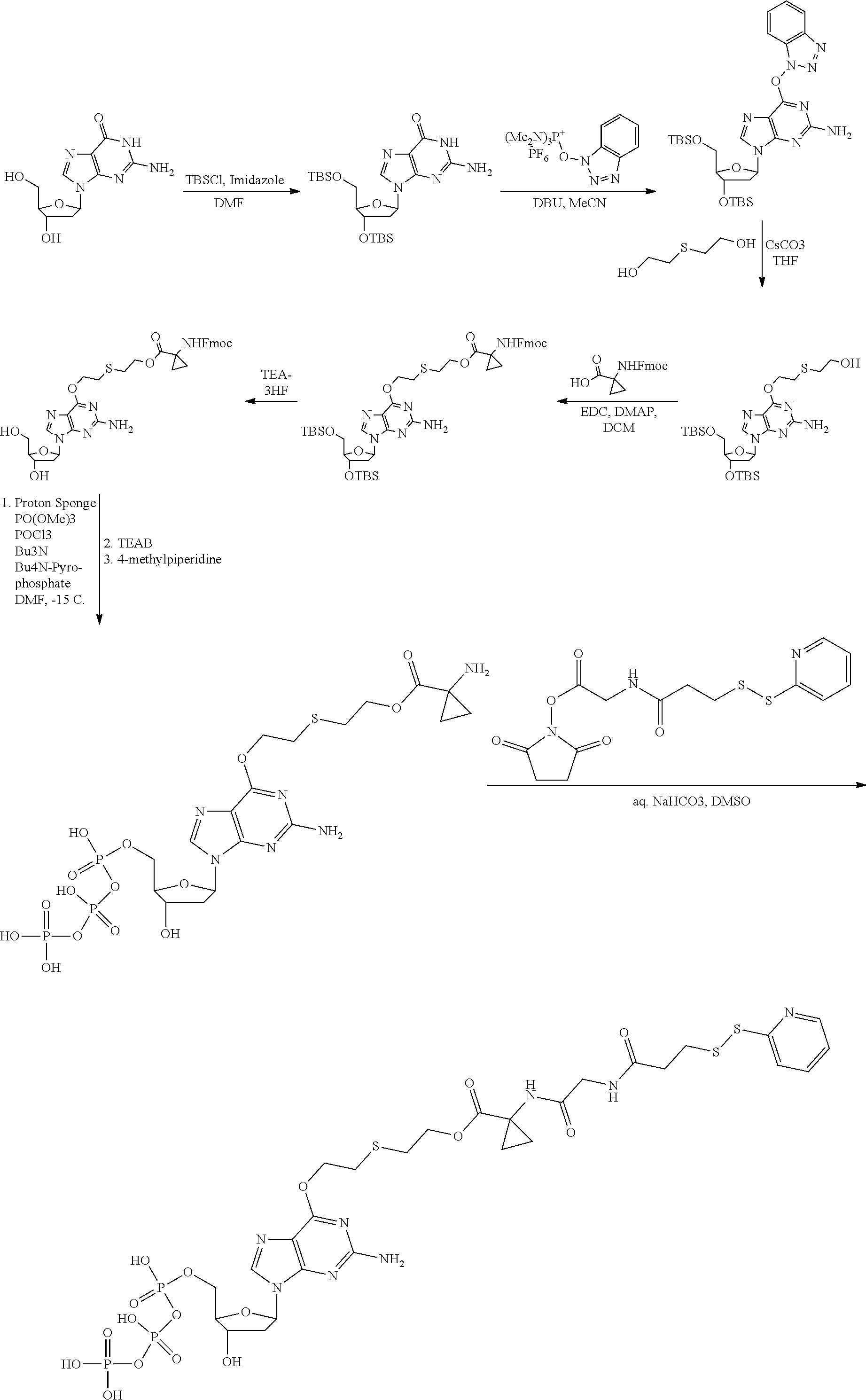

### Example 1: Preparation of Polymerase-Nucleotide Conjugates

Linker-Nucleotide Synthesis with Various R Groups on Amino Acid Ester

Modified nucleotides with L1 and the amino acid ester portion of L2 having various substitutions at the alpha carbon of the amino acid were synthesized according to the following reaction scheme:

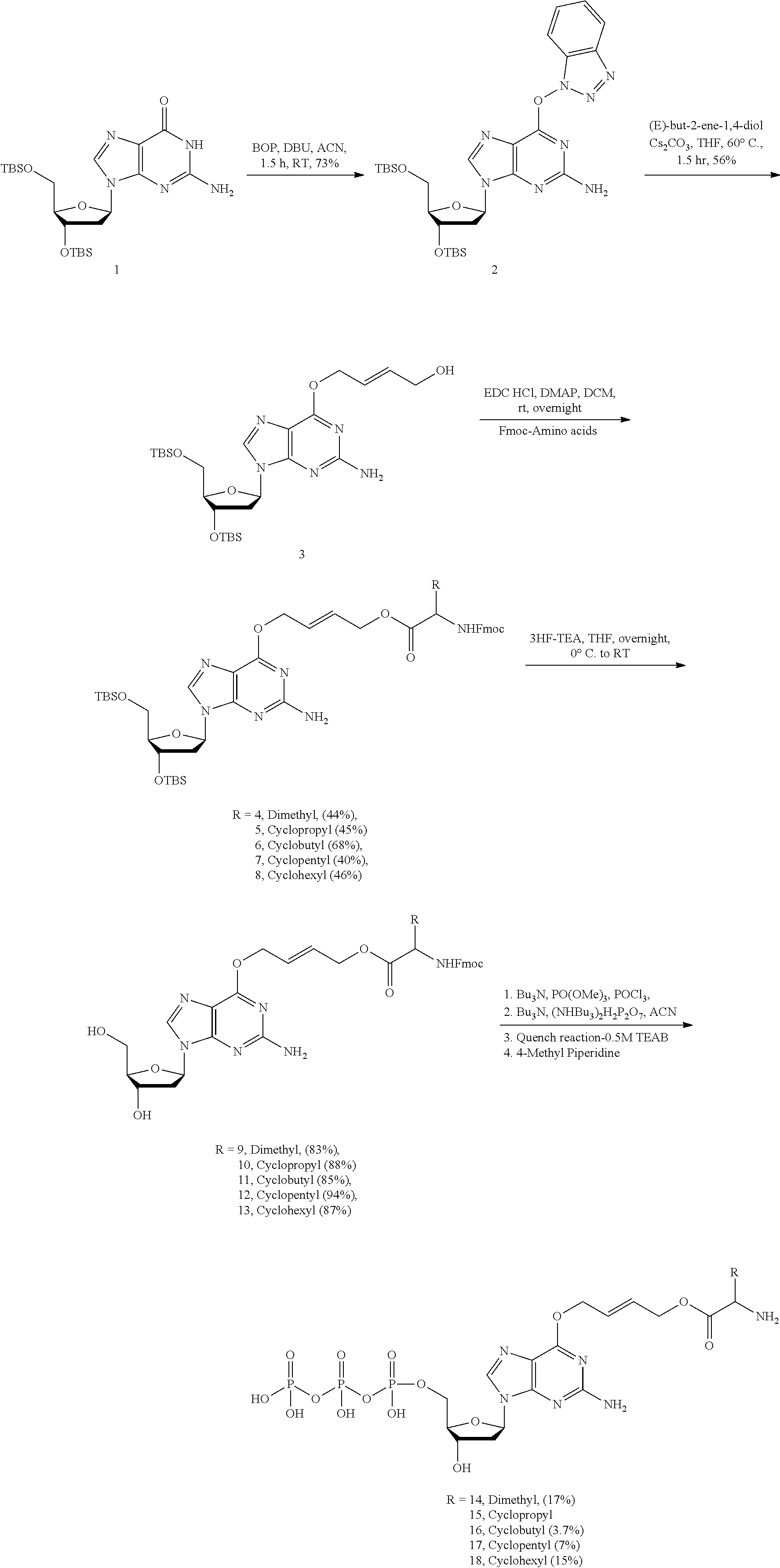

### Conjugation of L1 to O6 of Guanine

To a solution of the nucleoside derivative 1 (4 g, 8.08 mmol) in acetonitrile (16 mL), BOP (7.13 g, 16.14 mmol) and DBU (2.408 ml, 16.14 mmol) were added, and the mixture was stirred at room temperature for 1.5 hr. The mixture was then diluted with EtOAc (100 mL) and washed with deionized water (3×50 mL) followed by brine (30 mL). The organic layer was dried over anhydrous Na₂SO₄, filtered, and evaporated under reduced pressure. The obtained residue was dissolved in 3 to 4 ml DCM and then added to 50 mL of Hexane. Two layers were formed with an oily viscous layer on bottom and a turbid hexane layer on top. The hexane layer was decanted over sodium sulphate. The same hexane wash procedure was repeated two more times with the oily residue. The hexane layers were collected and evaporated under reduced pressure to obtain compound 2 as a white foam (3.63 g, 73%). This product used as such for the next reactions.

The compound 2 (3.62 g, 5.89 mmol) was dissolved in dry THF (30 mL), Cs₂CO₃ (3.84 g, 11.79 mmol) and E-Butene-1,4-diol (1.30 g, 14.72 mmol) were added, and the mixture was stirred at 60° C. or 1.5 hr. The reaction mixture was then diluted with EtOAc (100 mL), washed with deionized water (2×50 mL), and brine (25 mL). The organic layer was dried over anhydrous Na₂SO₄, filtered, and evaporated under reduced pressure. The crude material was purified by chromatography on a silica gel column using a 20-50% EtOAC/hexane gradient to give 3 (1.884 g, 56%) as a white foam.

### Addition of Amino Acid Ester

To a solution of the compound 3 (1 equiv) in dry DCM (0.2 M) was added DMAP (0.6 eq) and the Fmoc amino acid (1.2 eq) (corresponding to the dimethyl, cyclopropyl, cyclobutyl, cyclopentyl, or cyclohexyl R groups) under inert atmosphere at ambient temperature. The reaction mixture was cooled to 0° and then EDC·HCl was added slowly. The reaction mixture was allowed to stir for 16 hr at ambient temperature. The reaction solution was then diluted with additional DCM and extracted with water. The aqueous layer was then washed with more DCM (2×). The combined organic layer was then washed with sat. ammonium bicarb, dried over Na₂SO₄, and concentrated. The crude material was purified by chromatography on a silica gel column using a 10-35% EtOAC/hexane gradient.

### TBS Deprotection

To a 0.1 M solution of protected nucleosides (4-8) in dry THF at 0° C., was added 3HF-TEA (10 equiv) dropwise. The mixture was stirred for 16 hr while warming to ambient temperature. The reaction was quenched with the addition of a few drops of MeOH, and the solvents were removed under reduced pressure. The residue was diluted with DCM and washed with water. The aqueous layer was again extracted with DCM (1×). The combined organic layer was dried over anhydrous Na₂SO₄, filtered, and concentrated. The compound was purified by chromatography on a silica gel column using a 1-7% MeOH/DCM gradient. All the compounds were obtained as white foamy solids.

### Synthesis of Triphosphates and Fmoc Deprotection

Nucleoside analogue (9/11/12/13, 1 eq) was placed into a 10 mL round bottom flask equipped with a stir bar and tetrabutylammonium pyrophosphate was placed in a separate 5 mL conical tube. The two flasks were placed in a vacuum desiccator with P₂O₅ and allowed to dry under vacuum for at least 16 hr. Additionally, molecular sieves and three small round bottom flasks were placed in a drying oven for at least 16 hr. Two small flasks from the oven were charged with molecular sieves and flame-activated under vacuum. While these were cooling, the other small flask was attached to a Hickman distillation apparatus and flame dried. Upon cooling, the first two flasks were backfilled with nitrogen. Trimethyl phosphate and tributyl amine were then placed over the molecular sieves in the initial two flasks for drying. The Hickman distillation apparatus was then used to freshly distill POCl₃. The vacuum desiccator was purged with N₂ gas, and the flasks inside were then transferred to nitrogen balloons or the Schlenk line. Trimethyl phosphate (40 eq) was added to the nucleoside and the mixture was cooled at −5° C. To this nucleoside mixture was added dry tributyl amine (3 eq) followed by POCl₃ (2.1, 1.3, 1.5, 1.8 eq respectively) slowly via micro syringe. The combined mixture was stirred at −5° C. for 45 mins. After 45 min, the reaction mixture was treated with a mixture of tributylamine pyrophosphate (5 eq, 0.5 M in dry acetonitrile) and tributyl amine (6 eq). After 1 hour, the mixture was treated with triethylammonium bicarbonate (0.5 M, 1:2 of the total reaction volume) and allowed to stir at ambient temperature for 1 hour. Then, this reaction mixture was further treated with N-Methyl piperidine (⅕^{th} of total reaction volume) and stirred for 90 mins at ambient temperature followed by extraction with dichloromethane (2×). The aqueous layer was then purified by reverse phase HPLC (0.1 M triethylammonium acetate buffer/Acetonitrile, 4-47%, 0-15 min, flow 5 ml min-). Product containing fractions were pooled and lyophilized to provide desired product as a triethylammonium salt. The resulting solid was reconstituted in RNase Free DI water for further experiments.

### Addition of Glycine-L3 Portion of Linker

The Glycine amino acid portion of L2 and the L3 portion of the linker connecting L2 to a polymerase were then added to the compounds synthesized above according to the following reaction:

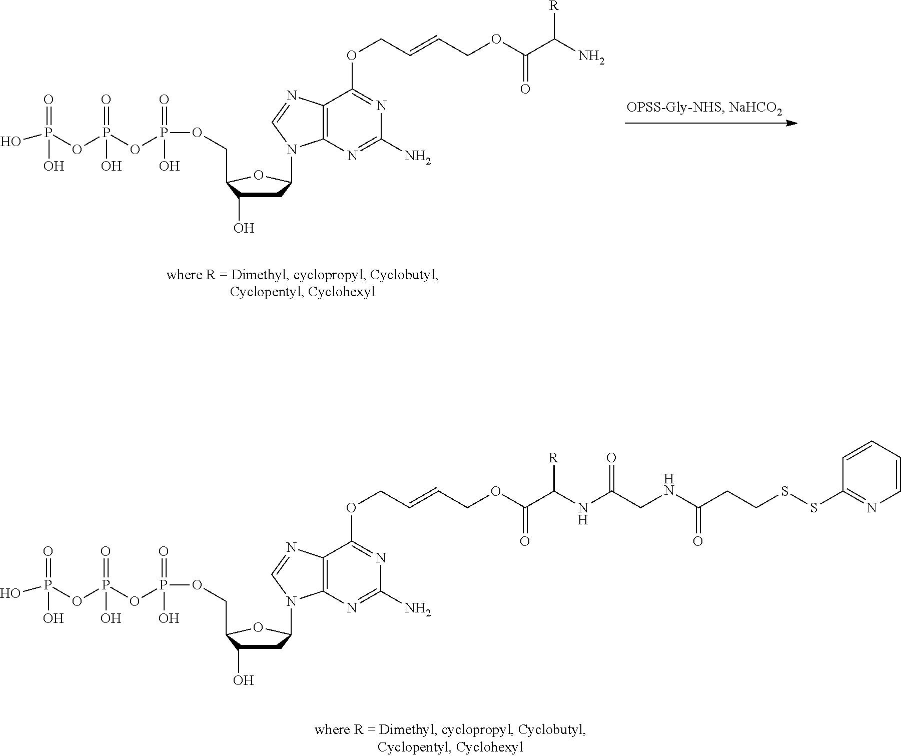

Specifically, 20 μL reactions were set with 2 mM nucleotide, 6 mM (3 eq.) OPSS-Gly-NHS ester and 0.1 M Sodium Bicarbonate (50. Eq) and an amide bond was formed by reaction with OPSS-Gly-NHS ester [2,5-dioxopyrrolidin-1-yl (3-(pyridin-2-yldisulfaneyl)propanoyl)glycinate)].

In order to compare the reactivity of the primary amine across these nucleotides (14-18), the amide bond formation rates were determined by injecting 10 μL of the reaction volumes on HPLC RP C-18 column at 10 mins and 50 mins respectively. (0.1 M triethylammonium acetate buffer/Acetonitrile, 4-47%, 0-20 min, flow 1 ml*min-) The % conversion to the product at each timepoint is tabulated in Table 1. As shown, the complete linker-nucleotide synthesis was successful for each of the amino acid ester R groups. A conjugate with a polymerase was formed as described in Example 1.

TABLE 1
Conversion of amines to amides at
reaction time 10 mins and 50 mins.

| | | % Amide | % Amide |
|---|---|---|---|
| | Amino | product | product |
| | acid ester | formed | formed |
| Nucleotide | R group | in 10 min | in 50 min |
| Compound 14 | Dimethyl (AiB) | 35 | 39 |
| Compound 15 | Cyclopropyl (ACC) | 83 | 85 |
| Compound 16 | Cyclobutyl (AC4C) | 87 | 94 |
| Compound 17 | Cyclopentyl (AC5C) | 56 | 58 |
| Compound 18 | Cyclohexyl (AC6C) | 41 | 44 |

Generation of Polymerase (TdT) Mutants with Various Attachment Positions for the Linker

An inducible plasmid expressing murine TdT with a single cysteine located at position 182 was produced (see Palluk et al., Nature Biotechnology, 2018 for complete protocol). Also see US Patent Publication No. 2019/0112627, “Nucleic Acid Synthesis and Sequencing Using Tethered Nucleoside Triphosphates” for further details of polymerase-nucleotide conjugate preparation, incorporated by reference herein in its entirety.

### Protein Expression and Purification of the Mutants

TdT expression was performed using BL21 (DE3) Gold cells (Agilent) in TB media containing antibiotics for resistance marker of the plasmid. An overnight culture of 50 mL was used to inoculate a 400 mL expression culture with 1/20 vol. Cells were grown at 37° C. and 200 rpm shaking until they reached OD 0.6. IPTG was added to a final concentration of 0.5 mM and the expression was performed for 16-20 h at 16° C. Cells were harvested by centrifugation at 8000 G for 10 min and resuspended in 20 mL buffer A (20 mM Tris-HCl, 0.5 M NaCl, pH 8)+5 mM imidazole. Cell lysis was performed using sonication followed by centrifugation at 30,000 G for 20 min. The supernatant was applied to a gravity column containing 1 mL of Ni-NTA agarose (Qiagen). The column was washed with 20 volumes of buffer A+40 mM imidazole, and bound protein was eluted using 4 mL buffer A+500 mM imidazole. The protein was concentrated to ˜0.15 mL with Vivaspin 20 columns (MWCO 10 kDa, Sartorius) and then dialyzed against 200 mL TdT storage buffer (100 mM NaCl, 200 mM K₂HPO₄, pH 6.5) over night using Pur-A-Lyzer™ Dialysis Kit Mini 12000 tubes (Sigma).

Ni-purified sample was applied to a HiTrap Q HP anion column. Protein was eluted with linear gradient from 100% Q Buffer A (100 mM NaCl, 20 mM K₂HPO₄, pH 6.5) to 100% Q Buffer B (1M NaCl, 20 mM K₂HPO₄, pH 6.5). SDS-PAGE analysis was used to identify fractions that contained TdT, these samples were pooled and concentrated.

### Attachment of Tethered Nucleoside Triphosphates to the Polymerase

To prepare TdT-nucleotide conjugates, a cleavable linker-nucleotide with a moiety capable of site specifically conjugating to a cysteine (i.e., maleimide) was first synthesized. Then, equal moles of TdT and linker-nucleotide were incubated overnight at 4° C. in 500 mM NaCl, 20 mM K₂HPO₄, pH 6.5. TdT conjugates were separated from unreacted linker-nucleotide using a S200 size exclusion column (Cytiva) pre-equilibrated in 20 mM Tris Acetate, 50 mM Potassium Acetate; pH 7.9.

### Example 2: Amino Acid Ester Cleavable Linkers

We have prepared and assayed several TdT-dNTP conjugates using different cleavable linkers to tether the nucleotide site-specifically to the polymerase. The use of a peptide bond in a linker results in a linker that can be cleaved by a protease. Cleavage of a peptide bond by a protease generates an amine and a carboxylic acid, both of which are charged under the buffer conditions that are typical for TdT activity. However, having charged functional groups persist on synthesized oligonucleotides can lead to deleterious effects during synthesis.

As an alternative, we explored the use of an amino acid ester in the linker to create the cleavable connection between nucleotide and TdT. Cleavage of an ester generates an alcohol as the charge-neutral cleavage product.

### Ester-Containing Linker Conjugates

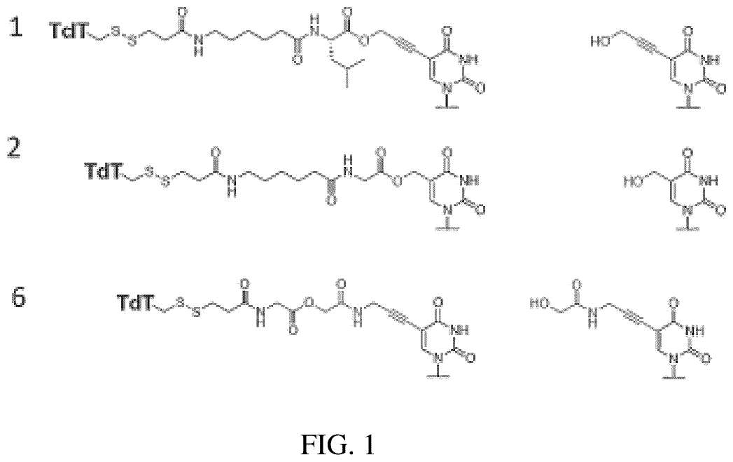

We initially designed two classes of ester-containing linkers that could be cleaved by an esterase to leave an alcohol-containing scar on the nucleobase. One is based on a hydroxypropargyl scar (Linker 1) and includes a cleavable leucine amino acid ester, and the other on a smaller hydroxymethyl scar (Linker 2) and includes a cleavable glycine amino acid ester. The two amino acid ester dTTP analogs (Linkers 1 and 2, FIG. 1; synthesized by Jena Bioscience) were attached to cysteine-reactive crosslinkers and conjugated to TdT.

First, we tested the impact of an incorporated nucleotide with an alcohol scar left by linker cleavage on continued extension during polynucleotide synthesis.

### Toleration of Hydroxymethyl Scar During Synthesis

To compare the extension kinetics of addition of a conjugate to the 3′ end of an unscarred nucleotide as compared to a hydroxymethyl scarred nucleotide, we performed a two cycle synthesis, first adding a dTTP conjugate to a natural DNA primer, followed by cleavage of the polymerase from the dTTP leaving a hydroxymethyl scar, then adding a dTTP conjugate to the DNA primer comprising the hydroxymethyl scarred nucleotide at the 3′ end.

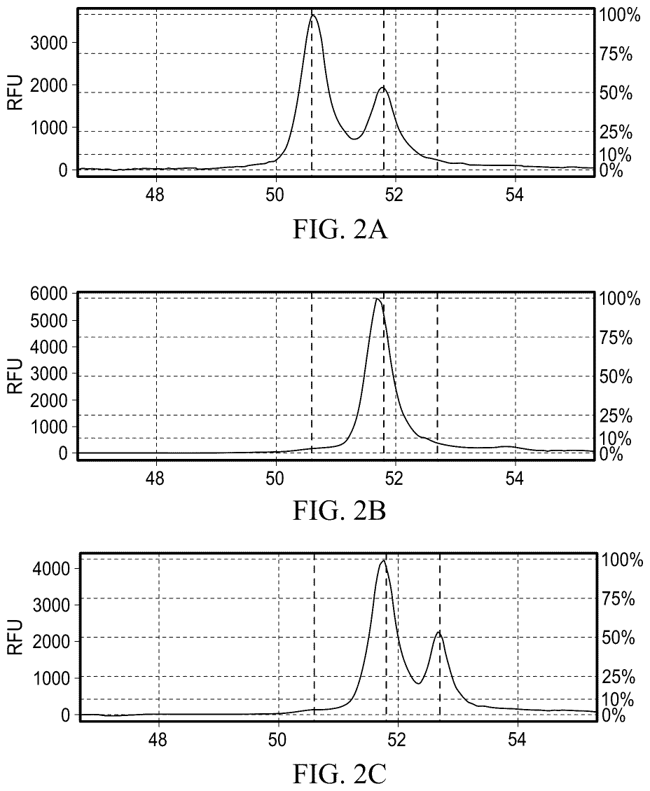

As shown in FIG. 2A, natural DNA primer exposed to a dTTP conjugate for 1 second results in ˜35% extension yield. The reaction was allowed to proceed to completion (FIG. 2B), with subsequent cleavage of the linker yielding a DNA primer comprising a hydroxymethyl scarred nucleotide at the 3′ end. Exposure of the scarred primer to the dTTP conjugate for 1 second again results in ˜35% extension yield, identical to the yield observed with natural DNA (FIG. 2C).

As the extension rate of the hydroxymethyl scarred DNA by the conjugate was identical to the extension rate of natural DNA, the ester-containing linker leaving an alcohol scar on the nucleotide is acceptable for DNA synthesis. The alcohol generated by the ester cleavage is a charge-neutral cleavage product, allows for unperturbed nucleotide addition, and is an improvement over protease cleavage which leaves charged nucleotide scars that negatively impact oligo synthesis.

### Ester-Containing Linkers

Next, we tested whether TdT-dTTP conjugates with linkers comprising an peptide and leaving a hydroxypropargyl scar (Linker 1—leucine amino acid ester) and a hydroxymethyl scar (Linker 2—glycine amino acid ester) could successfully incorporate dTTP onto the 3′ end of an oligonucleotide.

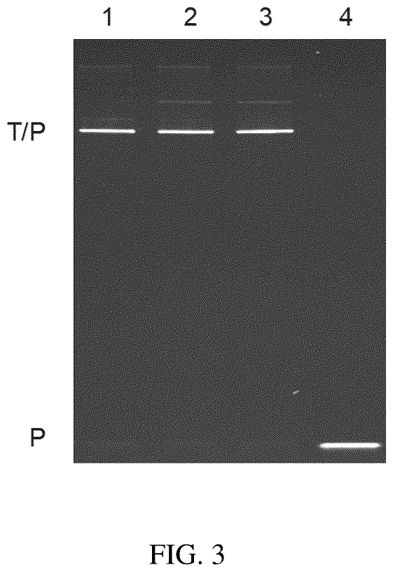

An ssDNA primer was extended for 60 seconds with 1) a Linker 1 conjugate, 2) a Linker 2 conjugate, 3) a Linker 2 conjugate (replicate), and 4) no conjugate. Upon incubation with an ssDNA primer, TdT-dTTP conjugates containing either linker formed a covalent primer-extension complex with >95% yield in under a minute, as measured by a gel shift assay on SDS-PAGE (FIG. 3). T/P: TdT/DNA complex. P: ssDNA primer (unbound). Bands above the complex are products with more than 1 base added to the primer.

As shown, the conjugates comprising amino acid ester cleavable linkers 1 and 2 are successfully added to the 3′ end of the oligonucleotide and are suitable for oligonucleotide synthesis.

### Esterase Screening and Enzymatic Cleavage

We screened several commercially available enzymes to identify an esterase to cleave conjugates made with Linkers 1 and 2. The serine protease Proteinase K, which is known to possess esterase activity (Barthel et al., Enhancing terminal deoxynucleotidyl transferase activity on substrates with 3′ terminal structures for enzymatic De Novo DNA synthesis. Genes (Basel). 2020; 11: 1-9.) cleanly cleaved both linkers to alcohol products. The oxypropargyl ester Linker 1 was cleaved completely in 2 minutes. The shorter oxymethyl ester Linker 2 required 7.5 minutes at 40° C. for complete cleavage. Thus, the amino acid ester-containing linkers in the polymerase-nucleotide conjugates, which have already shown to have good oligonucleotide incorporation kinetics, can also be successfully cleaved using a protease comprising an esterase activity.

Linker 2 dT(10) Synthesis

Shown above, we observed that the minimal hydroxymethyl scar left by Linker 2 is well-tolerated during oligonucleotide synthesis. To further explore, we synthesized a 10 mer poly T (dT(10)) at the end of a starter oligo using dTTP conjugates with Linker 2.

Synthesis of dT(10) using the dTTP conjugate with Linker 2 had an excellent coupling yield, with deletions well below the detection limit of 1%/step. However, the insertion rate was approximately 2.5%/step, which is undesirably high. This high insertion rate could be due to spontaneous cleavage of the ester. Because some esters are known to be labile, we had previously tested the stability of our amino acid ester containing linkers and discovered that both linkers 1 and 2 decompose upon heating. When the ester hydrolyzes, free nucleotides are released and can be incorporated by the TdT moiety of a primer-TdT complex, or an initial extension by a free polymerase, causing a second nucleotide to be added during the coupling step. The high insertion rate therefore could be due to spontaneous cleavage of the ester.

High insertion rate: 2.5% at pH 6.5; 0.5% at pH 7.9.

### Linker Instability Assay

Since the cleavage could be due to base-catalyzed hydrolysis, we tested the stability of the amino acid ester linker conjugates at a range of pH values from 8.5 to 6.5.

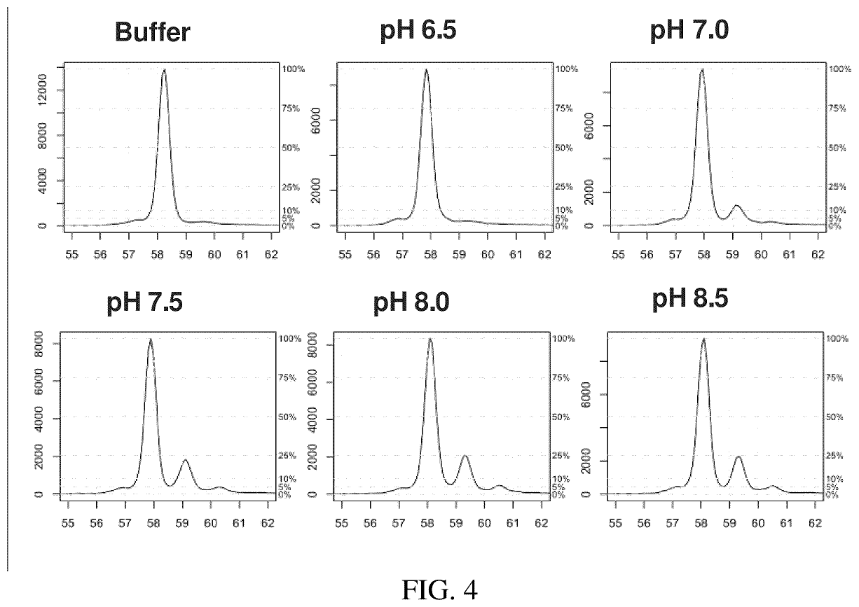

Specifically, dTTP conjugates comprising linker 2 were stored overnight at pH 6.5, pH 7.0, pH 7.5, pH 8.0, pH 8.5, or in buffer only as a control. A primer extension was performed incorporating the incubated dTTP conjugates into a primer. The resulting extension product was then run on a capillary electropherogram. The results are shown in FIG. 4, with insertions indicated by the second peak at ˜59. Insertions indicate the presence of free dNTPs in the conjugate solution. Linker 2 conjugates release dNTPs when stored at pH>6.5. However, at pH 6.5, we found a significantly reduced release of nucleotides by the conjugate.

dT(10), dT(100), and dT(200) Synthesis at pH 6.5

Next, we performed a new dT(10) synthesis using the linker 2 conjugates at pH 6.5. The insertion rate for this synthesis dropped to less than 1%/step, below the detection limit of the assay.

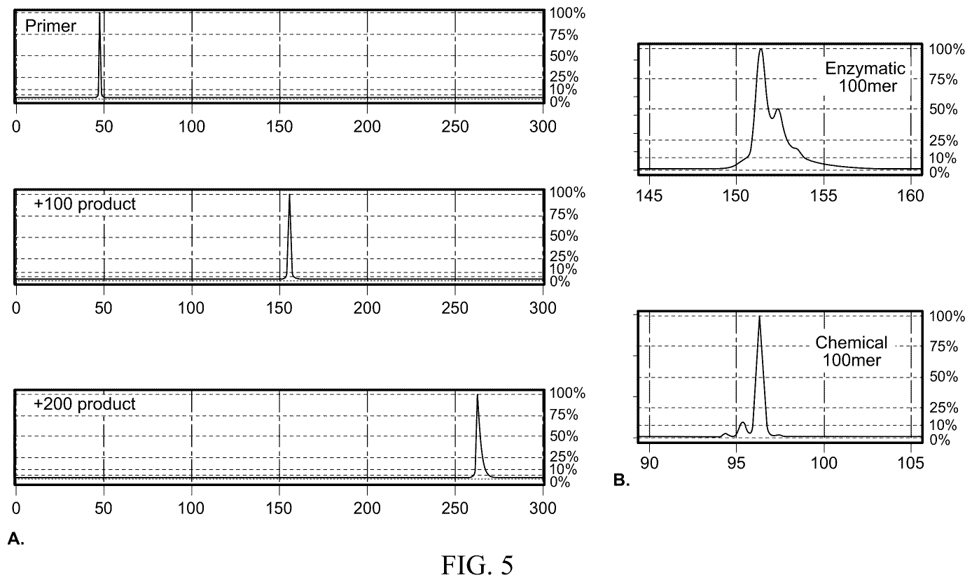

To measure low error rates more accurately, we increased the synthesis length to dT 100 and 200 mers to improve the limit of detection of synthesis errors. Using the linker 2 dTTP conjugates at pH 6.5, we synthesized dT 100 and 200mers onto the 3′ end of a 60 pb primer. There resulting dT 100 and 200 mers include hydroxymethyl scars. The synthesis proceeded with a low deletion rate of <0.1%/step and an insertion rate of ˜0.5%/step for both syntheses (FIG. 5, Part A).

For comparison to chemical synthesis, we ordered two dT 100mers from IDT (synthesized without capping to enable comparison of deletions), and assayed the chemically synthesized oligos via capillary electrophoresis. An enlarged view comparison between the enzymatically synthesized dT 100 oligo above and a 100 mer dT generated via chemical synthesis as measured via capillary electrophoresis is shown in FIG. 5, Part B. The most abundant species in both syntheses is +100 nt. The predominant type of errors are insertions (+101 nt) in the enzymatic product and deletions (+99 nt) in the chemical product. Enzymatic extension shows a lower deletion rate than chemical synthesis.

These results show that enzymatic synthesis using our conjugates has a deletion rate that is exceptionally low. Chemical oligonucleotide synthesis never achieves deletion rates below 0.1%/step, so by this metric our enzymatic synthesis approach is already superior. Further, the stepwise yields of the 100mer and 200mer were essentially identical, indicating that the yield did not fall as the synthesis proceeded. By contrast, chemical oligo synthesis typically sees increasing deletions as the synthesis gets longer. These results validate that a fully enzymatic oligonucleotide system will enable synthesis of longer oligos than is possible using chemical synthesis.

### Example 3: A, C, T, and G Conjugates with Protease Ester Cleavable Linker

We next tested a set of A, C, T, and G conjugates each having a protease ester cleavable linker to synthesize an oligonucleotide with a full set of nucleotides. For these conjugates we used an amino acid ester linker (linker 6) as shown in FIG. 1.

Linker 6 was assembled into linker-nucleotides by coupling an easy-to-synthesize ester-containing linker-COOH to the commercially available aminopropargyl dNTPs (Linker 6; FIG. 1). We purchased the ester-containing linker-COOH from WuXi AppTec (using non-SBIR funds) and contracted with MyChem LLC (San Diego) to couple it to aminopropargyl dNTPs, yielding the full set of Linker 6 nucleotides (A, C, G, T).

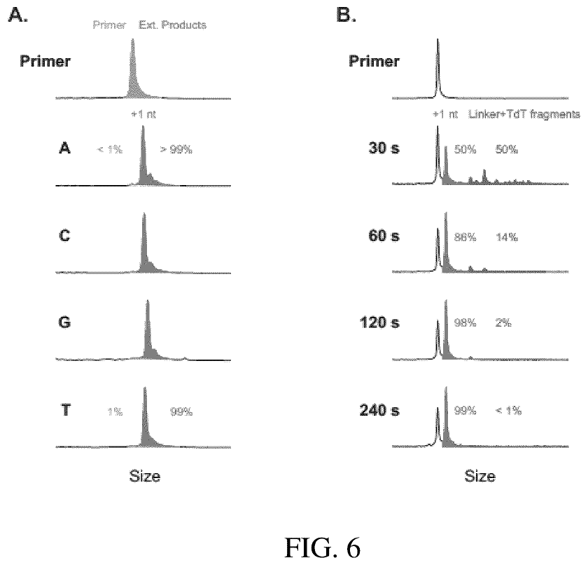

We then prepared conjugates from each linker nucleotide and tested their extension and cleavage kinetics. Specifically, an oligonucleotide primer was exposed to TdT-dATP, -dCTP, -dGTP, and -dTTP conjugates comprising linker 6 for 1 minute, followed by a 4 minute cleavage of the linker with Proteinase K. The cleaved extension product was then assayed via capillary electrophoresis. As shown in FIG. 6, Panel A, all four linker 6 dNTP conjugates showed >99% coupling yield in under 1 minute.

Next we performed a cleavage time course assay of a TdT-dTTP conjugate comprising linker 6 and incorporated onto a primer oligonucleotide. Specifically, coupling of the conjugate was performed for 1 second and the complex was exposed to Proteinase K for 30 seconds, 60 seconds, 120 seconds, or 240 seconds. The extension product was then assayed via capillary electrophoresis. As shown in FIG. 6, Panel B, the amino acid ester linker (linker 6) is >99% cleaved by Proteinase K in under 4 minutes.

Thus, we have demonstrated successful oligonucleotide synthesis using all four nucleotide conjugates comprising amino acid ester linkers and efficient cleavage of the linker with a protease comprising esterase activity (protease K) to separate the polymerase from the incorporated nucleotide.

However, we still observe a peak indicating spontaneous nucleotide insertions occur during the coupling reactions, which can be attributed to the instability of this ester linker, as demonstrated above.

dT(10) Synthesis

To test the performance of these conjugates further, we first synthesized a dT(10) oligo and analyzed the products by capillary electrophoresis. The coupling yield was excellent, with deletions <1%/step, below the limit of detection. As expected based on the observed insertions above, the conjugates comprising the linker 6 ester showed a high level of insertions, ˜3.5%/step. Thus, there is a need to improve ester stability to mitigate spontaneous cleavage of the linker. Furthermore, improving ester stability may allow for extended coupling time to further reduce the deletion rate.

### Example 4: ACC Ester Improves Linker Stability

As shown above, amino acid esters can be successfully cleaved by a protease comprising esterase activity (Proteinase K) to facilitate cleavage of the polymerase from the nucleotide after incorporation, leaving a neutral alcohol scar on the nucleotide, which does not hinder oligonucleotide synthesis. However, amino acid esters in linkers 2 and 6 are unstable and spontaneously cleave, leading to unwanted insertions during oligonucleotide synthesis. The linker 2 amino acid ester is a glycine analog that is unsubstituted at the alpha carbon. Here, we test whether addition of an aliphatic or bulky substituent to the alpha carbon of the amino acid ester improves the stability of the ester. Exemplary side group substitutions to improve stability are shown in FIG. G.

The stability of conjugates with varying cleavable linkers (glycine ester vs. ACC ester) was assayed using the following protocol:

A master mix (MM) was prepared with 20 mM tris acetate pH 7.9, 50 mM potassium acetate, 100 μM cobalt(II) acetate, and 100 nM DNA oligo substrate. To initiate the addition reaction, the MM was mixed 1:1 with a solution of the corresponding TdT-dNTP conjugate (2 μM solution in 20 mM tris acetate pH 7.9, 50 mM potassium acetate, and 0.1% Tween-20). The mixture was then incubated at room temperature for 5 minutes before quenching with EDTA (to a final EDTA concentration of 32 mM). At this time, samples were split for incubation at various temperatures. After 4 hours of incubation, samples were diluted 10-fold in HiDi containing 20 mM DTT. These diluted samples were then analyzed by capillary electrophoresis.

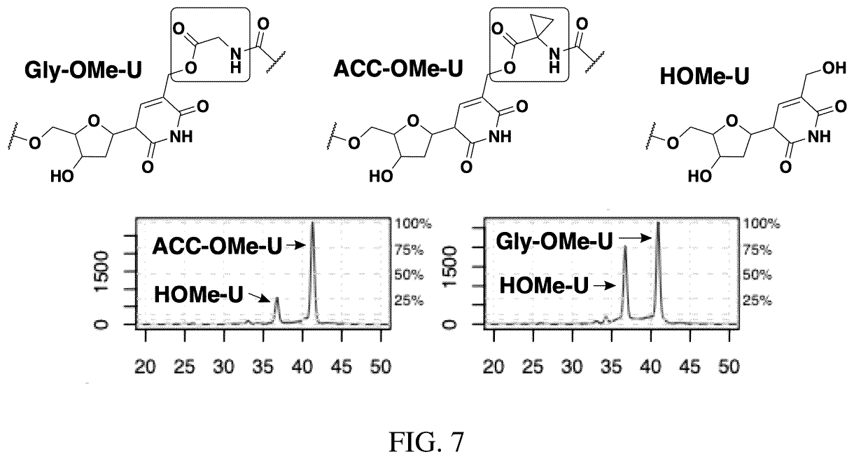

Upon examining the stability of the glycine ester linkage to the ACC ester linkage after 60 minutes of exposure to a temperature of 45° C. (FIG. 7), it is clear that hydrolysis of the ACC ester is less significant than with the corresponding glycyl ester. We suspect this stabilization is the result of the hyperconjugation effect of the cyclopropyl group.

### Example 5: Peptide Adjacent to ACC Ester Improves Enzymatic Cleavage

We next determined the rate of ProK-mediated linker cleavage for the ACC ester linkage.

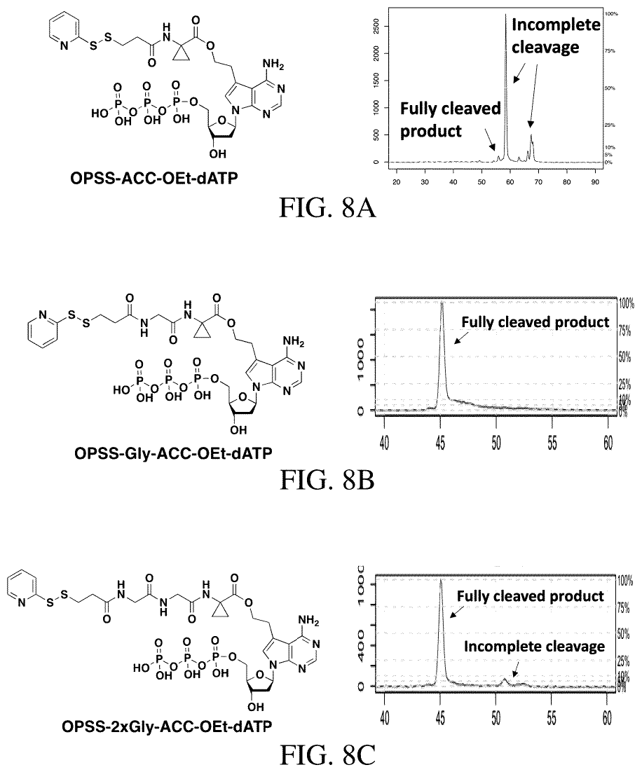

First, a conjugate with an ACC ester linkage as shown in FIG. 8A was incorporated into an oligo substrate as follows: A master mix (MM) was prepared with 20 mM tris acetate pH 7.9, 50 mM potassium acetate, 100 μM cobalt(II) acetate, and 100 nM DNA oligo substrate. To initiate the addition reaction, the MM was mixed 1:1 with a solution of the corresponding TdT-dNTP conjugate (2 μM solution in 20 mM tris acetate pH 7.9, 50 mM potassium acetate, and 0.1% Tween-20). The reaction was allowed to incubate for 5 min before quenching by the addition of EDTA (40 mM final concentration).

Next, we cleaved the ACC ester linkage as follows: The quenched addition reaction was then mixed 1:1 with a solution of ProK (40 U/mL final concentration) and EDTA (40 mM final concentration). Aliquots of the ProK reaction were removed and quenched at various time points by dilution in HiDi with Pefabloc, and subsequently analyzed by capillary electrophoresis.

FIG. 8A shows the cleavage products observed by capillary electrophoresis after 60 seconds of ProK treatment. As shown, very little complete cleavage product results from proK treatment of the ACC ester linker in the OPSS-ACC-OEt-dATP conjugate, indicating that the ACC ester linker structure is a poor substrate for ProK. Since cleavage of the linker is a critical step of conjugate-based oligonucleotide synthesis, we explored various modifications to improve cleavage activity of the ACC ester via ProK.

Among various possible alterations to the linker structure, we investigated the effect of incorporating one or more amino acids adjacent to the amine of the ACC ester in the linker. Linkers including one or two glycines bound to the amine of the ACC ester (OPSS-Gly-ACC-OEt-dATP and OPSS-2XGly-ACC-OEt-dATP, respectively) as shown in FIG. 8B and FIG. 8C were added to dATP. After preparing polymerase-nucleotide conjugates with these linkers, we incorporated the conjugate into an oligo substrate and cleaved the ACC ester linkage as described above for the ACC ester conjugate without glycine residues. The resulting products were subsequently analyzed by capillary electrophoresis. FIG. 8B and FIG. 8C shows the cleavage products observed by capillary electrophoresis after 60 seconds of ProK treatment. As shown, the ACC ester linker with a single glycine residue is cleaved by ProK to completion after 60 seconds (FIG. 8B), while the ACC ester linker with two adjacent glycine residues shows nearly complete cleavage after 60 seconds (FIG. 8C). Therefore incorporation of one or more amino acid residues adjacent to the stabilized protease ester significantly improves the kinetics of ProK cleavage to remove the polymerase from the incorporated nucleotide during oligonucleotide synthesis.

In conclusion, we show that addition of one or more amino acids to the L2 structure adjacent the amino acid ester improves kinetics of enzymatic cleavage of the linker by a protease comprising esterase activity.

### Additional Kinetics of ACC Ester—Glycine Linker ProK Cleavage

We further explored the ProK-mediated linker cleavage kinetics for the 1X Gly and 2X Gly ACC ester linkers.

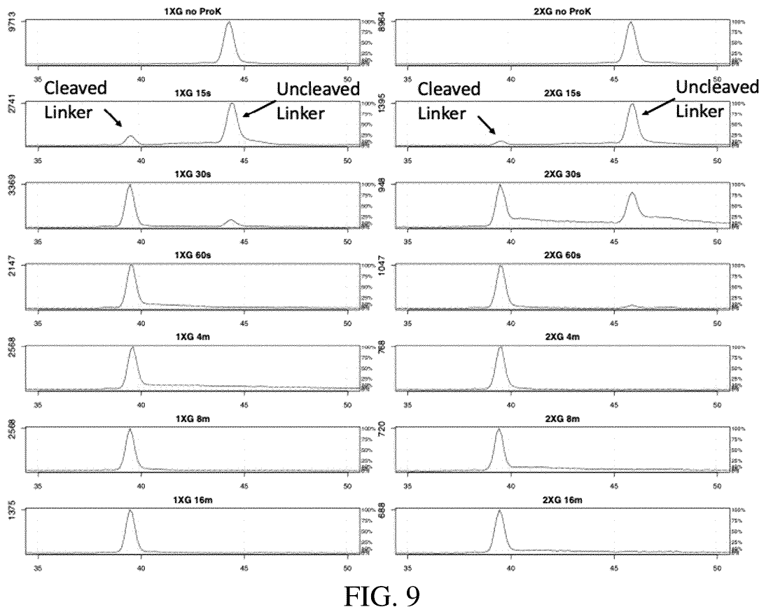

A short ssDNA oligo labelled with FAM was extended 1 nucleotide with a dATP conjugated to terminal deoxynucleotidyl transferase (TdT) via a linker having an aminocyclopropyl carboxy ethyl group and either one (1XG) or two (2XG) glycines. Following the extension reaction, the extended DNA was incubated with either ProK, or no ProK as a negative control, and quenched with pefabloc after 15 seconds (s), 30 s, 60 s, 4 minutes (m), 8 m, or 16 m. DTT was added to the analytical solution to remove any protein not cleaved from the linker. The cleaved and uncleaved DNA fragments were analyzed using capillary electrophoresis (CE). Fragment shifts in the electropherograms were observed to determine the size of the fragment, and thus the extent of linker cleavage. A fragment shifted to the left in the electropherogram is a smaller fragment, and thus indicates the presence of a cleaved linker. Cleaved and uncleaved linker peaks are annotated in FIG. 9.

The electropherograms shown in FIG. 9 indicate that cleavage of the 1XG linker is completed within 60 seconds whereas cleavage of the 2XG linker is completed within 4 minutes. This data shows that ProK is capable of cleavage of linkers having an aminocyclopropyl carboxy ethyl group (ACC amino acid ester) and either one (1XG) or two (2XG) glycines as a substrate. Furthermore, the data shows that the rate of linker cleavage by ProK can be modulated by changing the number of amino acids adjacent to the amino acid ester within the linker.

In conclusion, a single glycine residue adjacent to the amino acid ester has improved cleavage kinetics compared to two glycine residues adjacent to the stabilized amino acid ester in the L2 group. However, both linkers have significantly improved ProK clevage kinetics as compared to a linker with no amino acids adjacent to the stabilized amino acid ester in the linker.

### Nucleotide Addition/Oligo Synthesis

Next, we investigated the effect of each of the three L2 groups of our linkers assayed above (ACC, Gly-ACC, and 2XGly-ACC) on the kinetics of nucleotide incorporation by the corresponding TdT-nucleotide conjugate.

A master mix (MM) was prepared with 20 mM tris acetate pH 7.9, 50 mM potassium acetate, 100 μM cobalt(II) acetate, and 100 nM DNA oligo substrate with a CCC 3′ end. To initiate the addition reaction, the MM was mixed 1:1 with a solution of the corresponding TdT-dNTP conjugate (2 μM solution in 20 mM tris acetate pH 7.9, 50 mM potassium acetate, and 0.1% Tween-20). The reaction was quenched at various time points by the addition of EDTA and ProK. The resulting mixture was then diluted in HiDi for fragment analysis by capillary electrophoresis.

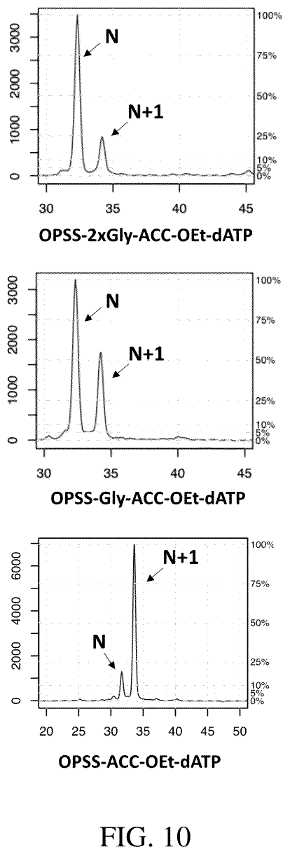

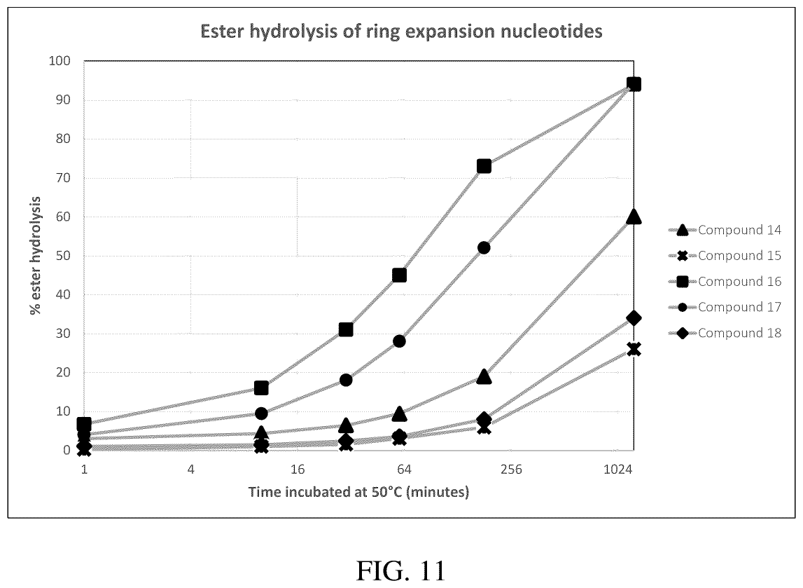

FIG. 10 shows the results of conjugate addition to the primer 3.8 seconds after addition of the conjugate for each of the TdT-nucleotide conjugates. The data demonstrates the stepwise removal of a single glycine, moving from 2 to 1 to zero glycines in L2, progressively results in increased kinetics of nucleotide incorporation. Although conjugate incorporation reactions for all 3 conjugates proceed relatively rapidly in view of the 3.8 second timepoint, a single amino acid adjacent to the amino acid ester in the linker may be preferred to optimize linker cleavage and conjugate incorporation speed.

In conclusion, we have engineered the L2 component of the cleavable linker for rapid protease activity and nucleotide addition. We have found that additional amino acids linked to the amine of ACC facilitate these rapid reactions, with a single additional glycine having the fastest cleavage kinetics. The additional peptide bonds do not result in free amines derived from off-target protease cleavage. Furthermore, the conjugation efficiency of the linker nucleotides we have developed is extremely high.

These improvements have resulted in the TdT-dNTP conjugates that support the synthesis of long, high quality oligonucleotides with very short cycle times via rapid nucleotide addition, rapid deprotection, and benign deprotection conditions.

### Example 6: Ring Expansion/ACC Variant—Ester Stability

As shown above use of a cyclopropyl R group on the amino acid ester confers increased stability to the ester group in the linker, minimizing unwanted spontaneous cleavage that could lead to insertions during oligo synthesis. Here, we explore other amino acid ester R groups to determine whether they also confer stability to the ester bond in the linker before it is cleaved by proK, and whether they are also suitable for conjugate addition to an oligonucleotide via TdT.

### Linkers Tested

To test the stability of the ester conferred by a ring expansion series and other substitutions at the R group of the amino acid ester of the L2 portion of the linker, we synthesized modified nucleotides comprising L1 and L2 groups with different R groups on the amino acid ester and subjected these to high pH conditions. Specifically, the following nucleotide linker compound was synthesized as described in Example 1 with dimethyl, cyclopropyl, cyclobutyl, cyclopentyl, and cyclohexyl R groups:

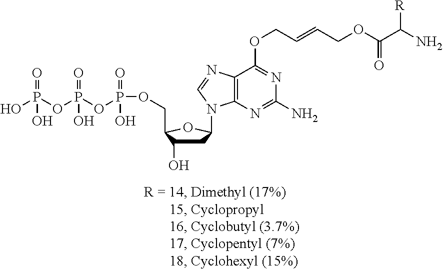

### Ester Stability of Compounds 14-18

In order to compare stability of the ester bond across the five analogues (compounds 14-18), the hydrolysis rate of these analogs was studied in TP8 buffer at 50° C. over a 20 hr timecourse. 3 mM solutions of the nucleotide analogues (14-18) in 1×TP8 (24 μL, pH 8) buffer were incubated at 50° C. 4 μL aliquots were taken out at 1 min, 10 min, 30 min, 1 hr, 3 hr and 20 hr time points and neutralized with 8 μL of KP 6.5 buffer and frozen at −80° C. The samples were then thawed and analyzed by analytical RP-HPLC. (0.1 M triethylammonium acetate buffer/Acetonitrile, 4-90%, 0-14 min, flow 1 ml min⁻¹). The % area of the hydrolyzed product peak relative to the total area of the starting product and hydrolyzed product peaks was used to determine hydrolysis rate.

A plot of the ester hydrolysis of each of the nucleotide analogues (14-18) over time at 50° C. is shown in FIG. 11.

Ester Stability of Linker after Incorporation into Starter DNA

Next, we determined ester stability for the ring expansion series conjugates after incorporation into an oligonucleotide by incubating the extended oligonucleotide at 50° C. for various amounts of time.

Polymerase nucleotide conjugates for each of the ring expansion series (compounds 14-18) were synthesized as described above. Concentrated TdT conjugate stocks were diluted to 0.2 mg/mL with 1×TP8+0.1% P20. The diluted conjugates were then mixed 1:1 with a solution of 50 nM starter DNA and 100 μM Co in 1×TP8 (10 μL+10 μL) to initiate the extension reaction at the following final concentrations: 25 nM DNA, 50 μM Co, 0.05% P20, 1×TP8 and at ambient temperature. After 4 min, 40 μL of 20 mM EDTA was added to quench the reactions. The combined mixtures were then incubated at 50° C. At the 1 hr, 4 hr, and 16 hr timepoints, 10 μL aliquots were removed from the reaction mixtures and frozen in a −80° C. freezer until they were thawed for analysis at the same time. 1 μL of each aliquot was diluted with 9 μL of analytical solution (75% HiDi containing DNA ladder and 20 mM DTT) and analyzed by capillary electrophoresis. For comparison a control sample (Allyl G) where the linker for the extended DNA product had been completely removed by a protease was used.

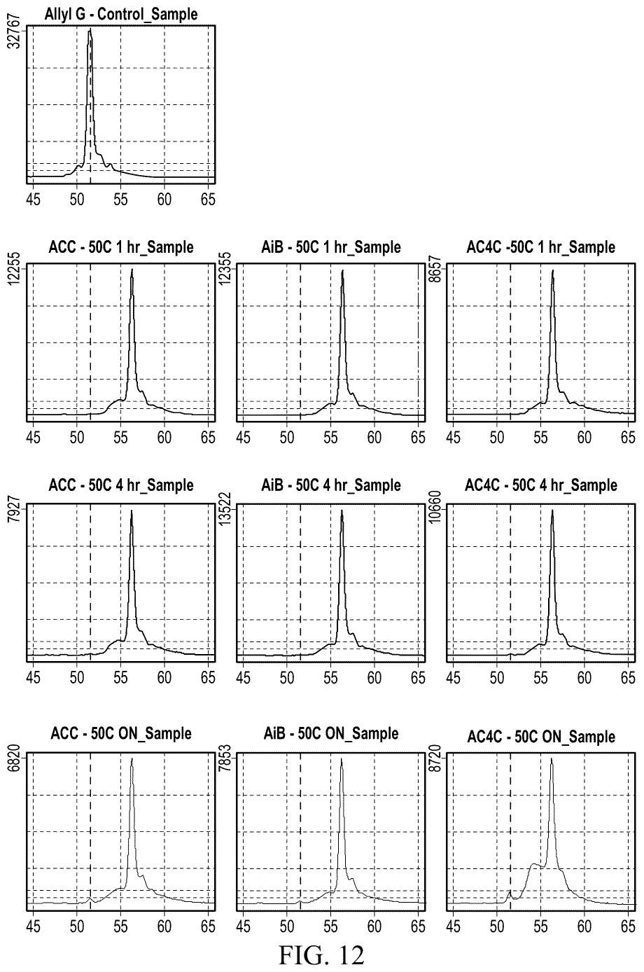

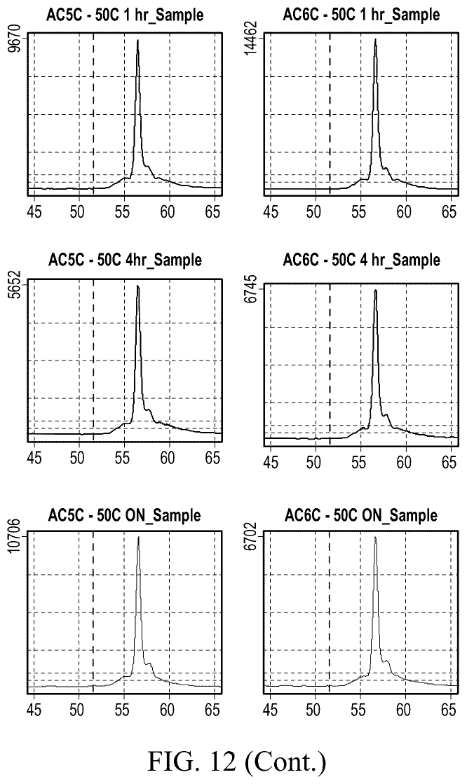

Results are shown in FIG. 12. The dotted reference line marks the position of the Allyl G control sample.

### CONCLUSION

The linkers on the ring expansion series (ACC, AiB, AC4C, AC5C, and AC6C) were minimally hydrolyzed after 16 hr incubation at 50° C. and displayed only small differences across the series, consistent with the stability of the ACC amino acid ester. As conjugates will typically only experience a few minutes of incubation at 37° C. for a standard synthesis, the linkers on entire ring expansion series have acceptable hydrolytic stability for single nucleotide extensions.

Furthermore, an optimized R group can be used based on the teachings herein to optimize the balance between ester stability and rate of cleavage by a protease comprising esterase activity. This can be done using one of the R groups shown in FIG. G, or a similar substituted amino acid ester, to achieve acceptable stability accompanied by increased linker cleavage kinetics.

### OTHER EMBODIMENTS

It is to be understood that the words which have been used are words of description rather than limitation, and that changes may be made within the purview of the appended claims without departing from the true scope and spirit of the present disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the present disclosure.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A conjugate comprising a polymerase, a nucleotide and a cleavable linker attached to the polymerase and the nucleotide, wherein the cleavable linker comprises an amino acid ester.

2. The conjugate of claim 1, wherein the amino acid ester is attached to an amino acid.

3. The conjugate of claim 2, wherein the amine group of the amino acid ester is bound to the amino acid.

4. The conjugate of claim 3, comprising a peptide of at least 2, at least 3, at least 4, or at least 5 amino acids bound to the amine group of the amino acid ester.

5. The conjugate of claim 2, wherein the amino acid is selected from the group consisting of: alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

6-7. (canceled)

8. The conjugate of claim 1, wherein the cleavable linker is bound to the alpha-phosphate, sugar, or nucleobase of the nucleotide.

9. The conjugate of claim 1, wherein the amino acid ester is represented by: 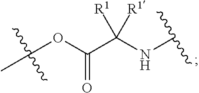
wherein R¹ and R^{1′} are each independently selected from hydrogen and an optionally substituted C₁₋₆ alkyl, or are optionally taken together with the atom on which they are attached to form an optionally substituted C₃-C₇ carbocyclic ring.

10. The conjugate of claim 9, wherein the amino acid ester is represented by a compound selected from the group consisting of:

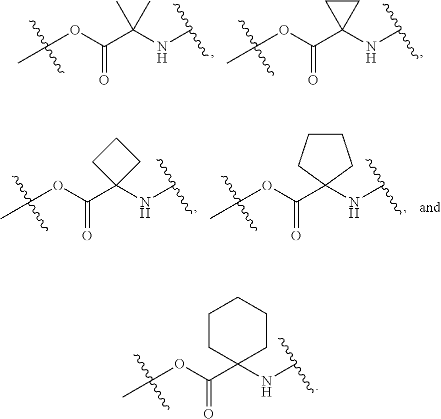

11. The conjugate of claim 1, wherein the linker comprises the structure: 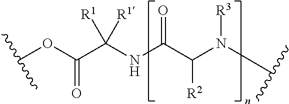
wherein R¹ and R^{1′} are each independently selected from hydrogen and an optionally substituted C₁₋₆ alkyl or are optionally taken together with the atom on which they are attached to form an optionally substituted C₃-C₇ carbocyclic ring;
each R² is an optionally substituted group independently selected from the group consisting of hydrogen, C₁₋₆ alkyl, phenyl, C₁-C₆ carbocyclic ring and 3-7 heterocyclic ring;
each R³ is hydrogen or optionally substituted C₁₋₆ alkyl; and
n is 1, 2, 3, 4 or 5.

12-17. (canceled)

18. The conjugate of claim 1, wherein the linker comprises the structure:

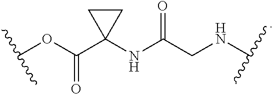 [SMILES: *NCC(=O)NC1(C(=O)O*)CC1]

19. The conjugate of claim 1, comprising the structure: 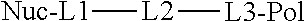
wherein:
Nuc is the nucleotide;
Pol is the polymerase;
L1 is a first portion of the linker connecting the nucleotide to L2;
L2 is a second portion of the linker represented by: 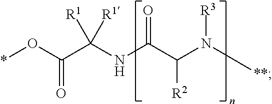
wherein R¹ and R^{1′} are each independently selected from an optionally substituted C₁₋₆ alkyl, a halogen, or are optionally taken together with the atom on which they are attached to form an optionally substituted C₃-C₇ carbocyclic ring;
each R² is an optionally substituted group independently selected from the group consisting of hydrogen, C₁₋₆ alkyl, phenyl, C₁-C₆ carbocyclic ring and 3-7 heterocyclic ring;
each R³ is hydrogen or optionally substituted C₁₋₆ alkyl;
n is 0, 1, 2, 3, 4 or 5;
wherein * indicates the attachment point of L2 to L1; and ** indicates the attachment point of L2 to L3;
wherein L² is cleavable; and
L³ is a linker connecting pol to L².

20. The conjugate of claim 19, wherein
L¹ is selected from the group consisting of a bond, an optionally substituted C₁₋₁₂ alkylene chain, C₄-C₂₀ polyethylene glycol, an optionally substituted C₂₋₁₂ alkenylene chain, and a C₂₋₁₂ alkynylene chain, wherein 1-6 methylene units of L¹ are optionally and independently replaced with —O—, —N(R^{b})—, —N═C(H)—, —C(O)—, —S—, —S(O)—, —S(O)₂—, optionally substituted phenylene, or optionally substituted cyclopropylene.

21. The conjugate of claim 20, wherein L¹ comprises: 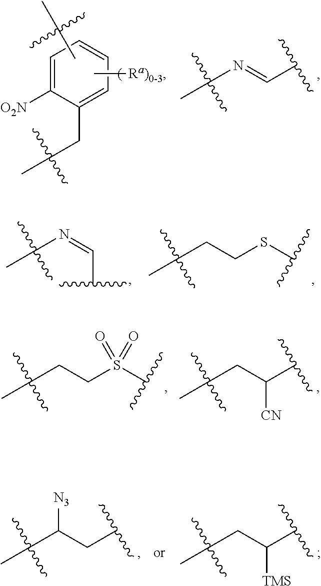
wherein each R^{a} is independently selected from the group consisting of halogen, hydroxyl, cyano, optionally substituted C₁₋₆ alkyl, and optionally substituted C₁₋₆ alkoxy.

22. The conjugate of claim 19, wherein L² comprises an amino acid ester selected from the group consisting of:

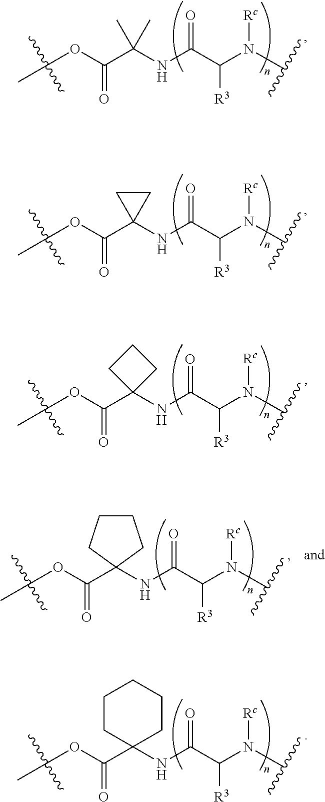

23. The conjugate of claim 22, wherein L² is represented by:

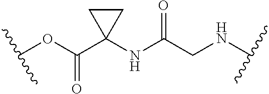 [SMILES: *NCC(=O)NC1(C(=O)O*)CC1]

24. The conjugate of claim 19, wherein L¹ is bound to the nucleobase of the nucleotide.

25. The conjugate of claim 24, wherein L¹ is bound to the nucleobase at an oxygen or nitrogen involved in base pairing.

26-36. (canceled)

37. A method of synthesizing a polynucleotide, comprising:
incubating a polynucleotide with the conjugate of claim 1.

38-46. (canceled)

47. A method of synthesizing a polynucleotide, comprising:
(a) incubating a nucleic acid with a first conjugate of claim 1 under conditions in which the polymerase catalyzes the covalent addition of the nucleotide of the first conjugate onto the 3′ hydroxyl of the nucleic acid to make a first extension product;
(b) cleaving the cleavable linkage of the linker, thereby releasing the polymerase from the extension product to de-shield the 3′ hydroxyl end of the first extension product;
(c) incubating the extension product with a second conjugate of any one of claims 1-36 under conditions in which the polymerase catalyzes the covalent addition of the nucleotide of the second conjugate onto the 3′ end of the first extension product, to make a second extension product;
(d) repeating steps (b)-(c) on the second extension product multiple times to produce an extended nucleic acid of a defined sequence.

48. (canceled)

49. A modified nucleotide comprising a cleavable linker, wherein the cleavable linker comprises an amino acid ester.

50-81. (canceled)
